(19) United States
(12) Patent Application Publication
(10) Pub. No.: US 2013/0041047 A1
(43) Pub. Date: Feb. 14, 2013

(54) Urinary Methylation Markers for Bladder Cancer

(71) Applicants: Torben Falck Ørntoft, Silkeborg (DK); Lars Dyrskjøt Andersen, Odder (DK); Jørgen Thomas Reinert, Risskov (DK); Charlotte Modin, Risskov (DK); Philippe Lamy, Aarhus C (DK)

(72) Inventors: Torben Falck Ørntoft, Silkeborg (DK); Lars Dyrskjøt Andersen, Odder (DK); Jørgen Thomas Reinert, Risskov (DK); Charlotte Modin, Risskov (DK); Philippe Lamy, Aarhus C (DK)

(73) Assignee: AROS APPLIED BIOTECHNOLOGY AS, Aarhus N (DK)

(21) Appl. No.: 13/486,527

(22) Filed: Jun. 1, 2012

Related U.S. Application Data
(60) Provisional application No. 61/491,912, filed on Jun. 1, 2011.

(51) Int. Cl. C12Q 1/68; A61P 35/00; A61K 35/00; C40B 30/04; C40B 20/00

(52) U.S. Cl. 514/789; 506/9; 506/2; 435/6.11

(57) ABSTRACT

A method of detecting bladder cancer is described using hypermethylated urinary markers.

### RELATED APPLICATION DATA

This application claims the benefit of provisional application Ser. No. 61/491,912 filed Jun. 1, 2011 which is hereby incorporated by reference in its entirety.

### FIELD

The present invention relates to methods of detecting bladder cancer in an individual using urinary methylation markers.

### BACKGROUND

Cancer of the urinary bladder is the fifth most common neoplasm among the populations of industrialized countries.

Epigenetics is the study of mitotically and/or meiotically heritable changes in gene function that cannot be explained by changes in DNA sequence. Several forms of epigenetic regulation are known, including histone modifications and DNA methylation. DNA methylation occurs during critical normal processes like development, genomic imprinting, and X-chromosome inactivation. Alterations in epigenetic control have been associated with several human pathologic conditions including cancer. See, Egger G, et al., Epigenetics in human disease and prospects for epigenetic therapy, Nature 2004; 429:457-63. CpG sites are sparsely distributed throughout the genome except for in CpG islands. See, Takai D, et al., Comprehensive analysis of CpG islands in human chromosomes 21 and 22, Proc. Natl. Acad. Sci. 2002; 99:3740-45; Gardiner-Garden M, et al., CpG islands in vertebrate genomes, J. Mol. Biol. 1987; 196:261-82. CpG dinucleotides outside CpG islands are generally hypermethylated in normal cells and undergo a substantial loss of DNA methylation in cancers.

CpG sites within CpG islands are usually in an unmethylated state permissive to transcription in normal cells, but become hypermethylated at certain promoters in cancers. Transcriptional inactivation by CpG island promoter hypermethylation is a well-established mechanism for gene silencing in cancer, including bladder cancer, and aberrant methylation is associated with stage, and grade of the tumors as well as recurrence rate and progression.

In 75% of all cases of urinary bladder cancer, the primary tumor will present as a non-muscle invasive tumor stage Ta or T1 (NMIBC). The remaining 25% of the cases will present with invasion of the bladder muscle, stage T2-4 (MIBC). Stage Ta bladder cancer is characterized by frequent recurrences after resection, in as many as 60% of patients. See Millan-Rodriguez F, et al., Primary superficial bladder cancer risk groups according to progression, mortality and recurrence. J Urol 2000; 164:680-4. Often one or more tumors will appear each year over an 8-10 years period without any progression, however, up to 25% will eventually develop an aggressive invasive phenotype. See Wolf H, Kakizoe T, et al. Bladder tumors, Prog Clin Biol Res 1986; 221:223-55.

Patients diagnosed with superficial bladder cancer are generally monitored over an extended time period with a cystoscope, which is extended into the bladder through the urethra. Such monitoring causes patient discomfort and is costly. Markers for bladder cancer which can be detected in patient urine would decrease the cost of monitoring and lessen the discomfort of patients. Markers which indicate the likelihood of cancer progression would have additional value in determining courses of treatment.

### SUMMARY

Hypermethylation of one or more of the urinary markers HOXA9, ZNF154, POU4F2, and EOMES indicates existence of urinary bladder cancer. Hypermethylation of TBX4 was not found to be a urinary marker for detecting the presence of bladder cancer, but was newly discovered to be associated with a likelihood of bladder cancer progression from stage Ta to stage T1 or T2, or another more advanced stage and is disclosed herein as a marker for bladder cancer progression. Disclosed is assaying a subject's urine sample whether one or more of the markers HOXA9, ZNF154, POU4F2, and EOMES is hypermethylated, for example within the promoter region, relative to the level of methylation in said markers in a control, or, relative to the methylation level of other or all genomic material, such as genomic DNA material or genomic DNA in the assay. Following the determination, the information can be used to initiate a monitoring program for subjects with hypermethylation of the relevant markers such as increasing frequency of cystoscopies if hypermethylation is observed or reducing monitoring for patients with no observed hypermethylation of the relevant markers. The determination can also be used to alter the course of treatment—for example, treating more aggressively (e.g. with cystectomy, chemotherapy or immunotherapy) because of the increased progression risk. The determination can be done using any assay technique, including those described herein, i.e., Infinium Array, bisulfate sequencing or Methylation-Sensitive High Resolution Melting. Monitoring programs and treatment methods are known to those of skill in the art.

According to certain aspects of the present disclosure, a method is provided for identifying bladder cancer in a subject or predicting a likelihood of a subject developing bladder cancer. According to one aspect, the method includes collecting urine from a subject, assaying genomic material in the urine for one or more of the markers HOXA9, ZNF154, POU4F2, and EOMES being hypermethylated relative to the level of methylation in said markers in a control representative of a subject. i.e. a control sample from a subject, who is negative for bladder cancer or relative to the level of methylation of the total genomic material, such as genomic DNA material or genomic DNA, in the assay, and wherein hypermethylation indicates bladder cancer in the subject.

According to a certain aspect, a determination is made by hybridizing the genomic material to an array of probes where the array is capable of determining the average percentage of methylation of the markers. According to an additional aspect, bisulfite sequencing is used in the determination of the average percentage of methylation of the markers. According to one aspect, following bisulfite sequencing, a high resolution melting analysis is performed.

According to certain aspects, methods described herein include determining whether any markers other than HOXA9, ZNF154, POU4F2, and EOMES are hypermethylated or hypomethylated in a tissue sample from the subject. According to one aspect, the tissue sample is obtained by performing a cystoscopy on the patient. According to one aspect, the tissue sample is obtained by performing a transurethral resection of a bladder tumor on the patient. According to an additional aspect, the markers are one or more of PTGDR; ZNF135; TBX4; ACOT11; PCDHGA12; CA3; CHRNB1; BRF1; SOCS3; PTGDR; or SCARF2. According to a still additional aspect, the one or more of the markers PTGDR; ZNF135; TBX4; ACOT11; PCDHGA12; or CA3 is hypermethylated relative to the level of methylation in the markers in the control; and/or one or more of the markers CHRNB1; BRF1; SOCS3; PTGDR; or SCARF2 is hypomethylated relative to the level of methylation in said markers in the control.

According to an additional aspect, hypermethylation of the markers is observed, and a monitoring program for the subject for bladder cancer development or progression or recurrence is undertaken. According to one aspect, hypermethylation of the markers is observed, and initiation of treatment, or a change in existing treatment regimens, is undertaken. According to one aspect, the array described herein with respect to the methods described herein is analyzed by establishing a threshold which reflects a significant level of methylation. According to one aspect, the assaying described herein includes amplification of portions of the markers HOXA9, ZNF154, POU4F2, and EOMES. According to a certain aspect, the amplification step includes use of primers targeting the methylated or unmethylated portions of the markers.

According to one aspect of the present disclosure, a method is provided for identifying, detecting, confirming, determining, diagnosing, or prognosing bladder cancer in a subject including assaying genomic material in urine from the subject for one or more of the markers HOXA9, ZNF154, POU4F2, or EOMES being hypermethylated relative to the level of methylation in respective HOXA9, ZNF154, POU4F2, or EOMES non-bladder cancer control markers or relative to the level of methylation of total genomic material in the assay; and wherein hypermethylation of one or more of the markers HOXA9, ZNF154, POU4F2, or EOMES indicates bladder cancer in the subject.

According to one aspect, the step of assaying includes hybridizing the genomic material to an array of probes where the array indicates the average percentage of methylation of the markers. According to an additional aspect, the step of assaying includes bisulfite sequencing to determine the average percentage of methylation of the markers. According to an additional aspect, a high resolution melting analysis is performed following bisulfite sequencing.

According to one aspect, methods described herein including determining whether markers other than HOXA9, ZNF154, POU4F2, and EOMES are hypermethylated or hypomethylated in a tissue sample from the subject. According to one aspect, the tissue sample is obtained by performing a cystoscopy on the patient. According to one aspect, the tissue sample is obtained by performing a transurethral resection of a bladder tumor on the patient. According to one aspect, the markers from the tissue sample are one or more of PTGDR; ZNF135; TBX4; ACOT11; PCDHGA12; CA3; CHRNB1; BRF1; SOCS3; PTGDR; or SCARF2.

According to a certain aspect, the one or more of the markers PTGDR; ZNF135; TBX4; ACOT11; PCDHGA12; or CA3 is hypermethylated relative to the level of methylation in respective control markers; or one or more of the markers CHRNB1; BRF1; SOCS3; PTGDR; or SCARF2 is hypomethylated relative to the level of methylation in respective control markers.

According to one aspect, hypermethylation of the markers is observed and the subject is monitored for bladder cancer development, recurrence or progression. According to one aspect, hypermethylation of the markers is observed and the subject is treated for bladder cancer. According to one aspect, the array described herein with respect to the methods described herein is analyzed by establishing a threshold which reflects a significant level of methylation. According to one aspect, the assaying described herein includes amplification of portions of the markers HOXA9, ZNF154, POU4F2, or EOMES. According to a certain aspect, the amplification step includes use of primers targeting the methylated or unmethylated portions of the markers.

According to certain aspects, methods are provided where hypermethylation of two or more of the markers HOXA9, ZNF154, POU4F2, or EOMES indicates bladder cancer in the subject. According to certain aspects, methods are provided where hypermethylation of three or more of the markers HOXA9, ZNF154, POU4F2, or EOMES indicates bladder cancer in the subject. According to certain aspects, methods are provided where hypermethylation of the markers HOXA9, ZNF154, POU4F2, or EOMES indicates bladder cancer in the subject.

According to certain aspects, the step of assaying in the methods described herein includes assaying for markers TWIST1 or VIM being hypermethylated relative to the level of methylation in respective TWIST1 or VIM non-bladder cancer control markers or relative to the level of methylation of total genomic material in the assay, and wherein hypermethylation of one or more of the markers TWIST1 or VIM indicates bladder cancer in the subject.

According to certain aspects, a method is provided for identifying bladder cancer in a subject including assaying genomic material in urine from the subject for the marker HOXA9 being hypermethylated relative to the level of methylation of HOXA9 in a non-bladder cancer control sample or relative to the level of methylation of total genomic material in the assay, and wherein hypermethylation of HOXA9 indicates bladder cancer in the subject. According to certain aspects, a method is provided for identifying bladder cancer in a subject including assaying genomic material in urine from the subject for the marker ZNF154 being hypermethylated relative to the level of methylation of ZNF154 in a non-bladder cancer control sample or relative to the level of methylation of total genomic material in the assay, and wherein hypermethylation of ZNF154 indicates bladder cancer in the subject. According to a certain aspect, a method is provided for identifying bladder cancer in a subject including assaying genomic material in urine from the subject for the marker POU4F2 being hypermethylated relative to the level of methylation of POU4F2 in a non-bladder cancer control sample or relative to the level of methylation of total genomic material in the assay, and wherein hypermethylation of POU4F2 indicates bladder cancer in the subject. According to a certain aspect, a method is provided for identifying bladder cancer in a subject including assaying genomic material in urine from the subject for the marker EOMES being hypermethylated relative to the level of methylation of EOMES in a non-bladder cancer control sample or relative to the level of methylation of total genomic material in the assay, and wherein hypermethylation of EOMES indicates bladder cancer in the subject.

### BRIEF DESCRIPTION OF THE FIGURES

FIG. 1 is a schematic representation of experimental setup (A) and a flow chart of the gene selection process (B).

FIG. 2 depicts methylation data from microarrays and MS-HRM based validation. A) Genes with differential methylation between normals and cancers. Normals: (n=6), Cancers: (n=50). B) MS-HRM validation of tumor markers. Normals: (n=8), Cancers: (n=55). A beta or methylation value of 0 means no methylation, whereas 1 means 100% methylation.

FIG. 3 depicts methylation data from microarrays and MS-HRM based validation. A) Genes with differential methylation between stages (CHRNB1, BRF1, SOCS3 and SCARF2) and a candidate marker of disease progression (TBX4). Normals: (n=6), Cancers: (n=50). B) MS-HRM validation of genes with differential methylation between stages (CHRNB1, BRF1, SOCS3 and SCARF2) and a candidate marker of disease progression (TBX4). Normals: (n=8), Cancers: (n=55). A beta or methylation value of 0 means no methylation, whereas 1 means 100% methylation.

FIG. 4 depicts methylation data from microarrays and MS-HRM based validation. A) Genes with differential methylation between normals and cancers. A beta value of 0 means no methylation whereas 1 means fully methylated. Normals: (n=6), Cancer: (n=50). B) MS-HRM validation of tumor markers. A methylation value of 0 means no methylation, whereas 1 means 100% methylation. Normals: (n=8), Cancers: (n=55).

FIG. 5 depicts data showing analytical validation by bisulfite sequencing of bladder tumor markers ZNF154 (A), HOXA9 (B), POU4F2 (C), and EOMES (D). The upper part of each panel provides a schematic representation of the transcription start site. The darker bar labeled IP indicates the Infinium probe annealing site and the lighter bars below the “IP” bar, labeled MSP represent MS-HRM primer binding sites. The numbers show the CpG sites in the sequence. The column on the right side lists the methylation status of the gene (above or below the cut point) reported by the Infinium array (U=unmethylated, M=methylated). On the left side, the sample type is given as normal or tumor. Each circle represents the average methylation of 10 to 12 clones. A hollow circle means no methylation, whereas a filled circle means 100% methylation.

FIG. 6 depicts data showing bisulfite sequencing of the bladder tumor markers CA3 (A), ACOT11 (B), PCDGHA12 (C), and PTGDR (D). The darker bar labeled IP indicates the Infinium probe annealing site and the lighter bars below the “IP” bar, labeled MSP represent MS-HRM primer binding sites. The numbers show the CpG sites in the sequence. The column on the right side lists the methylation status of the gene (above or below the cut point) reported by the Infinium array (U=unmethylated, M=methylated). On the left side, the sample type is given as normal or tumor. Each circle represents the average methylation of 10 to 12 clones. A hollow circle means no methylation, whereas a filled circle means 100% methylation.

FIG. 7 depicts data of bisulfite sequencing of the bladder tumor markers HIST1H4F (A), GRM4 (B), and SLC22A12 (C). The darker bar labeled IP indicates the Infinium probe annealing site and the lighter bars below the “IP” bars, labeled MSP represent MS-HRM primer binding sites. The numbers shows the CpG sites in the sequence. The right column right lists the methylation status of the gene (above or below the cut point) reported by the Infinium array (U=unmethylated, M=methylated). On the left, the sample type is given as normal or tumor. Each circle represents the average methylation of 10 to 12 clones. A hollow circle means no methylation, whereas a filled circle means 100% methylation.

FIG. 8 depicts data showing technical validation of the Infinium array by bisulfite sequencing. A) Sequence of the novel CHRNB1 tumor stage marker candidate. The shaded sequences are bisulfite sequencing primer annealing sites. The bold letters indicate CpG sites (n=15), which are numbered 1 to 15 and indicated above the sequence starting right after the forward primer's annealing site. The 50 nucleotides recognized by the Infinium probes are underlined. The italicized sequences are annealing sites for the MS-HRM primers used in the independent validation assay. The Infinium probes span CpG sites 7 and 8, whereas the MS-HRM amplicon includes CpG site 5 to 10. B) Bisulfite sequencing of 6 samples. The numbers above the figures indicate CpG site number, as in A C) Infinium array methylation percentage plotted as function of the bisulfite sequencing methylation percentage for the CHRNB1 gene (n=6). A hollow circle represents an unmethylated CpG site whereas a filled circle represents a 100% methylated CpG site.

FIG. 9 depicts data showing relative chromosomal distribution of differentially methylated CpG sites between normals and tumors. Distribution of CpG sites normalized to the total number of CpG sites on a given chromosome for |Δβ|>0.25 for CpG sites within CpG islands (black) and CpG sites outside CpG islands (gray).

FIG. 10 is a schematic flow chart illustrating the collection and analysis of urine samples as described herein.

FIG. 11 shows DNA methylation data associated with subsequent tumor recurrence within 24 months for patients without tumor but with methylation positive urine samples. Kaplan-Meier plots of recurrence free survival as a function of dichotomized methylation levels for ZNF154 (P<0.0001) (A), EOMES (P=0.0397) (B) HOXA9 (P=0.0009), POU4F2 (P=0.0001) (D), TWIST1 (P=0.0017) (E), and VIM (P=0).

FIG. 12 depicts data showing DNA methylation is associated with subsequent tumor recurrence within 60 months for patients without tumor but with methylation positive urine samples. Kaplan-Meier plots of recurrence free survival as a function of dichotomized methylation levels for ZNF154 (P<0.0001) (A), EOMES (P=0.0254) (B) HOXA9 (P=0.0024), POU4F2 (P=0.0001) (D), TWIST1 (P=0.0034) (E), and VIM (P=0.0001) (F).

FIG. 13 depicts data of examples of methylation levels at first visit and two subsequent visits for 5 patients with or without recurrences. Patients with two subsequent recurrences are shown in A and B. Patient with no recurrences at first control visit, but recurrence at a later control visits is shown in C and D. A patient with no subsequent recurrences is shown at E. PMR is the percentage of methylated reference. A value of 0% means no methylation and a value of 100% means fully methylated. A dark bar represents a visit with concomitant tumor and a gray bar represents a visit with no tumor.

### DETAILED DESCRIPTION

The novel urinary markers of methylation were found by analysis of a large number of samples by using microarray analysis for an initial identification followed by confirmation using Methylation-Sensitive High Resolution Melting (MS-HRM). Bisulfite sequencing was also performed on the urinary markers as an additional assay for marker methylation. Statistical correlations were determined as described below.

### Example I

Patient material: A total of 119 tissue samples from patients with bladder cancer but without other malignant disease, were analyzed by Infinium Array or Methylation-Sensitive High Resolution Melting (MS-HRM). Most patients provided metachronous tumors. The samples were obtained fresh from transurethral resection of bladder tumors from patients, embedded in Tissue-Tek (O.C.T) Compound (Sakura Finetek), and immediately snap frozen in liquid nitrogen. Normal bladder urothelium (for controls) was obtained from individuals who had benign prostate hyperplasia or bladder stones.

Samples were macro dissected (for tumor samples) or laser dissected (normal samples) to obtain a urothelial cell percentage of at least 75%. Sample composition was confirmed by H&E evaluation of sections cut before and after those used for extraction. Voided urine was collected from 115 bladder cancer patients (for evaluating urinary markers) and 59 individuals with benign prostate hyperplasia or bladder stones (for controls). Nineteen of the controls were stix positive for nitrite, indicating bacterial infection. Urine specimens were collected immediately before urinary cytology or cystoscopy, pelleted by centrifugation, and frozen at −80° C.

DNA Extraction and Bisulfite Modification:

Tissue DNA was extracted using the Puregene DNA purification kit (Gentra Systems, Minneapolis Minn.). One microgram (μg) of DNA extracted from fresh frozen tissue was bisulfite modified using the EZ-96 DNA methylation D5004 (Zymo Research, Irvine Calif.) for the Infinium array, or EpiTect (Qiagen, Inc., MA) for the MS-HRM, respectively. Urinary DNA was extracted using the Puregene DNA purification kit (Gentra Systems) according to the manufacturer's recommendations. Tissue and urine DNA purity was assessed using the OD 260/280 ratio.

Infinium Array:

One μg of DNA from each sample was whole genome amplified and hybridized overnight to Infinium Arrays, scanned by a BeadXpress Reader instrument (Illumina, Inc.) and data were analyzed by the Bead Studio Methylation Module Software (Illumina) and exported to Excel for further analysis. The CpG island status was obtained from Bead Studio. For each of the 27,578 probes the Infinium assay returns a beta value (β), which approximately corresponds to the average percentage of methylation in the sample analyzed. Illumina reports that the Infinium array is accurate with Δβ-values above 0.2. The Δβ cutoff value for differential methylation was conservatively set to ±0.25.

Cloning and Bisulfite Sequencing:

Primers for bisulfite sequencing of CpG island regions were designed using Methprimer and primer sequences are shown in Supplementary Table 1. PCR for cloning was carried out with the Accuprime™ Taq DNA Polymerase System (Invitrogen) according to the manufacturer's instructions, in a final volume of 25 microliters (μl) using 4 μl of bisulfite modified DNA as template. Amplification cycling temperatures can be seen in Supplementary Table 1, for each primer pair. PCR amplicons were gel purified using the QIAQUICK Gel Extraction Kit (Qiagen) and TOPO TA cloned for sequencing (Invitrogen) according to the manufacturer's instructions. Twelve random colonies from each gene were used for colony PCR in a final volume of 25 μl using the TEMPase Kit (Ampliqon) according to the manufacturer's instructions. Primers were M13 forward and M13 reverse from the TOPO TA Cloning Kit (Invitrogen). The sequencing reactions were analyzed in a 3130× Genetic Analyzer (Applied Biosystems).

Methylation Sensitive High Resolution Melting (MS-HRM):

Methylation-Sensitive High Resolution Melting (MS-HRM) was carried out in triplicate with 15 sets of primers (Supplementary Table 1) using 1.5 μl (15 nanograms (ng)) of bisulfite modified DNA as template in a final volume of 10 μl using the LightCycler™ 480 High Resolution Melting Master (Roche). Each plate included a no template control (NTC) and a standard curve (100%, 75%, 50%, 25%, 5%, and 0% methylated samples, CpGenome™ Universal Methylated DNA (from Millipore) diluted with unmethylated peripheral blood DNA. Melting curves were analyzed on a LightScanner (Idaho Technology Inc.).

RNA Purification and Gene Expression Microarray:

RNA was purified using the RNeasy Kit (Qiagen). The RNA integrity and RNA Integrity Number (RIN) was assessed with the 2100 Bioanalyzer (Agilent). 500 ng of RNA from each sample were loaded on a Human Exon 1.0 ST Arrays (Affymetrix). Microarray analysis and data handling was performed as is conventional, and described for example, in Dyrskjot L, et al., Identifying distinct classes of bladder carcinoma using microarrays, Nat Genet 2003; 33:90-96).

Data Analysis:

Genespring GX 10 software (Agilent) was used for Exon array analysis. Data was quantile normalized using ExonRMA16 software with transcript level core (17881 transcripts) and by using antigenomic background probes. The statistical analysis was performed with independent samples only, except for the two analyses of metachronous tumors. The independent tumor analysis included analysis of methylation in the Ta(stable) tumor group compared with the Ta(stable2) tumor group and the analysis of the methylation level in the Ta(prog) tumor group compared with the methylation level in the subsequent progressed tumor (T1 or T2-4). Ta(stable) and Ta(stable2) respectively consist of the first and second tumor from patients with a stable Ta disease. The second tumor is a recurrent tumor. Ta(prog) consists of Ta tumors from patients with subsequent progression to T1 or T2-4. When patients had several Ta tumors before the disease progressed to stage T1 or higher, the Ta tumor closest to the stage progression, i.e., the Ta tumor with the shortest timespan to the progressed tumor, was used in the analysis.

Gene Ontology (GO) and Ingenuity Pathway Analysis (IPA):

ene symbols of genes showing hypo or hyper methylation were used as input in GO analysis. The undivided list was submitted to IPA (2000-2008 Ingenuity Systems) and the data were analyzed to identify (adjusted for multiple testing by the Benjamini-Hochberg method) top network associated functions and Canonical pathways.

Statistical Analysis:

Stata 10 (Statacorp, Tex., USA) was used for analyzing methylation data from MS-HRM using the nonparametric Wilcoxon-Mann-Whitney test. The inter observer agreement coefficient (κ) was calculated for MS-HRM. The Infinium Array data was analyzed using nonparametric Wilcoxon-Mann-Whitney or Wilcoxon signed-rank test in R (see r-project.org) to evaluate differential methylation between independent groups (based on stage of progression) or related samples, respectively. As synchronous lesions were very similar in methylation only one from each patient was included for statistical calculation. There was no adjustment for multiple testing due to limited group sizes. The most interesting CpG sites were instead validated on an independent sample set. The Chi-squared test was used for evaluation of chromosomal distribution. Excel (Microsoft) was used for a two tailed student's t-test to evaluate different mRNA expression between groups and Pearson correlations to progression.

Genome Wide Methylation in Urinary Bladder Cancer:

The genome wide DNA methylation status of six normal urothelium samples and 50 urothelial carcinomas (UC) of the bladder, were first profiled using microarrays interrogating 27,000 known CpG sites. To study the methylation over time in single individuals, we analyzed metachronous tumors (two-three tumors from 18 patients). We subdivided patients with stage Ta bladder cancer into stable disease (named Ta(stable), or Ta(stable2)) when taken from the same patient) if no progression to higher stages was observed and progressing disease (named Ta(prog)), indicating the bladder cancer progressed from stage Ta to stage T1 or higher. The average CpG site methylation within CpG islands was increased (p=0.013; student's t-test) in the aggressive Ta(prog), T1 and T2-4 tumors, compared to normals and Ta(stable) tumors. Sites outside CpG islands measured a decrease (p=0.0095; student's t-test) in average CpG site methylation reaching 18.5% in the Ta(stable) group and 10.6% in the T2-4 tumor group compared to normal tissue. Using the Ta(stable) tumors as a reference group it was evident that the majority of changes in methylation occurred in the transition from normal to cancer. These findings are in concordance with other findings in cancer tissues compared to normal tissues.

Gene Specific Methylation Differences:

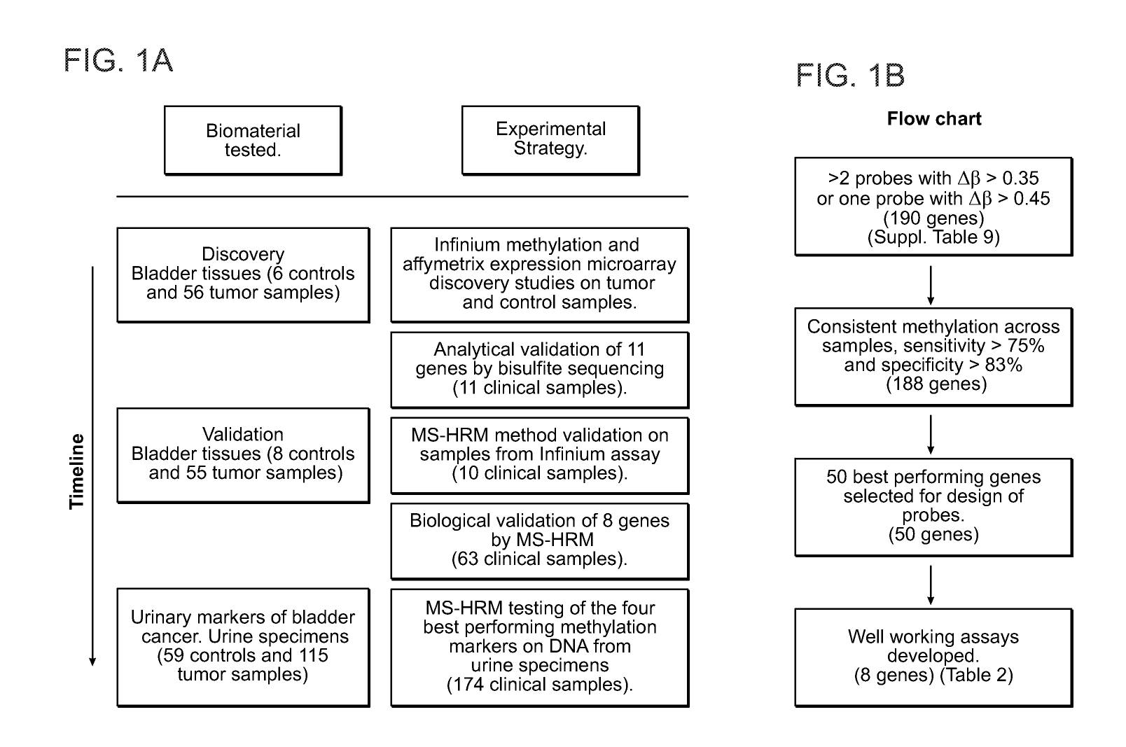

Table 1 below is a list of the 19 most highly differentiated methylated genes between controls and tumors, as well as genes selected as indicated in the flow chart depicted in FIG. 1B, validated alone by bisulfite sequencing (#) or by bisulfite sequencing and independent validation (*), and sorted by delta beta values (Δβ-values). ND indicates not determined. Δβ-values were calculated as average tumor methylation β-value minus average control methylation β-value. Pearson correlation coefficients between methylation and expression are shown. Infinium Array target ID, the presence of a CpG island, chromosome number, and distance of CG dinucleotides to transcription start site (TSS) are specified. Statistical analysis was performed using a two-sample Wilcoxon rank-sum (Mann-Whitney) test. Bold print indicates genes of special interest. Out of the 19 most differentially methylated genes between normals and tumors, eleven showed hypomethylation and eight hypermethylation in cancer.

TABLE 1

| | Δβ- | | Sensitivity | Specificity | Pearson | Distance | | CpG | Infinium |
|---|---|---|---|---|---|---|---|---|---|
| Gene | value | P-value | (%) | (%) | correlation | to TSS | Chr. | island | targetID |
| Most hypermethylated | | | | | | | | | |
| ZIC1 | 0.52 | <0.0001 | 100 | 83 | −0.08 | 171 | 3 | + | cg14456683 |
| ZNF154* | 0.52 | 0.0018 | 85 | 100 | −0.68 | 68 | 19 | + | cg21790626 |
| SPAG6 | 0.52 | 0.0001 | 96 | 83 | 0.05 | 361 | 10 | + | cg25802093 |
| MYCL2 | 0.50 | 0.0009 | 77 | 100 | ND^{$} | 6 | X | + | cg12537796 |
| HOXA9* | 0.50 | 0.0003 | 92 | 100 | −0.46 | 35 | 7 | + | cg07778029 |
| KCNA1 | 0.50 | 0.0009 | 92 | 83 | −0.16 | 148 | 12 | + | cg08832227 |
| ZNF154 | 0.50 | 0.0049 | 81 | 100 | −0.75 | 100 | 19 | + | cg08668790 |
| HSPA2 | 0.50 | 0.0004 | 96 | 83 | 0.07 | 850 | 14 | + | cg27120999 |
| Selected | | | | | | | | | |
| POU4F2* | 0.47 | 0.0004 | 92 | 100 | −0.13 | 38 | 4 | + | cg24199834 |
| HIST1H4F^{#} | 0.45 | 0.0005 | 92 | 100 | ND | 266 | 6 | + | cg08260959 |
| ACOT11* | 0.44 | 0.0004 | 92 | 100 | 0.33 | 192 | 1 | − | cg10266490 |
| EOMES* | 0.44 | 0.0004 | 88 | 100 | −0.01 | 1498 | 3 | + | cg15540820 |
| PCDHGA12* | 0.43 | 0.0001 | 96 | 100 | ND | 21 | 5 | + | cg07730329 |
| CA3* | 0.42 | 0.0001 | 88 | 100 | −0.09 | 123 | 8 | + | cg18674980 |
| PTGDR* | 0.39 | 0.0218 | 58 | 100 | 0.08 | 98 | 14 | + | cg09516965 |
| GRM4^{#} | −0.43 | <0.0001 | 96 | 100 | −0.19 | 476 | 6 | + | cg01962826 |
| SLC22A12^{#} | −0.46 | <0.0001 | 88 | 100 | −0.08 | 335 | 11 | + | cg07220939 |
| FTHL17 | −0.51 | <0.0001 | 96 | 100 | 0.25 | 478 | X | − | cg04515986 |
| KRTAP11-1 | −0.51 | <0.0001 | 96 | 100 | 0.19 | 114 | 21 | − | cg07014174 |
| MMP26 | −0.51 | <0.0001 | 100 | 83 | 0.10 | 113 | 11 | − | cg12493906 |
| ERAF | −0.51 | <0.0001 | 100 | 100 | 0.27 | 31 | 16 | − | cg02989940 |
| REG3G | −0.51 | <0.0001 | 96 | 100 | ND | 384 | 2 | − | cg00918005 |
| FFAR2 | −0.52 | <0.0001 | 100 | 100 | −0.05 | 245 | 19 | + | cg15479752 |
| CNTNAP4 | −0.52 | <0.0001 | 100 | 100 | −0.10 | 119 | 16 | − | cg06793062 |
| TNFSF11 | −0.52 | <0.0001 | 100 | 100 | 0.19 | 326 | 13 | − | cg21094154 |
| CNOT6 | −0.53 | =0.0001 | 96 | 100 | −0.25 | 835 | 5 | + | cg15241708 |
| EBPL | −0.54 | <0.0001 | 100 | 100 | −0.33 | 616 | 13 | + | cg20399252 |
| MAGEB6 | −0.62 | <0.0001 | 100 | 100 | 0.26 | 34 | X | − | cg10127415 |

Most hypomethylated
^{#}Validated by bisulfite sequencing.
*Validated by bisulfite sequencing and independent biological validation.
^{$}Not determined.

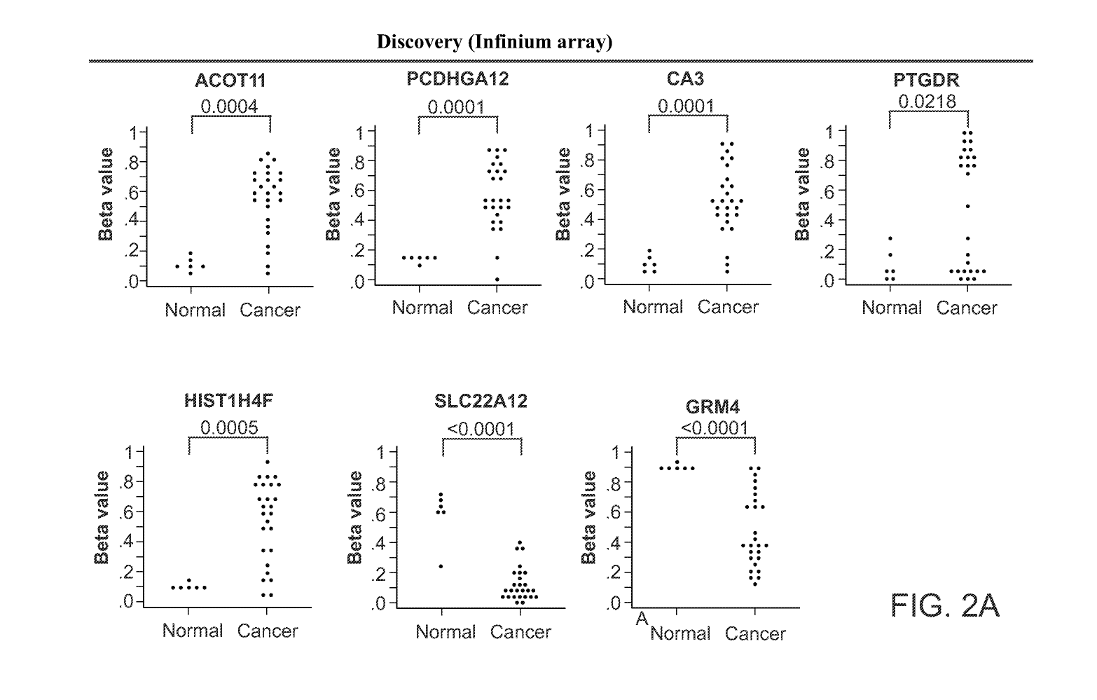

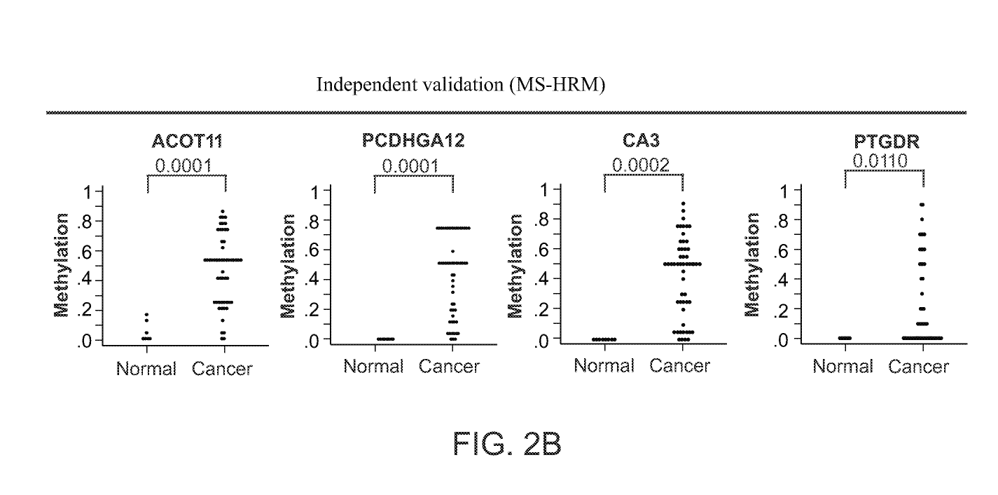

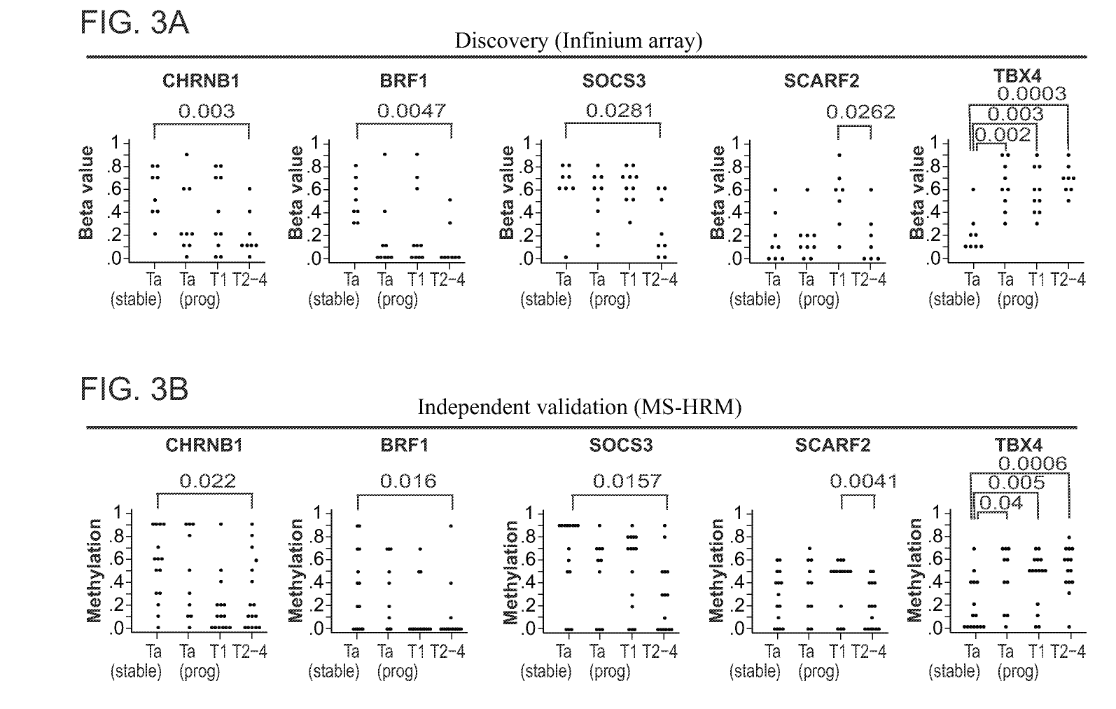

Nine other genes showed a high sensitivity and specificity when comparing normal and cancer (see flow chart for gene selection at FIG. 1B). Eleven genes (ZNF154, HOXA9, POU4F2, EOMES, CA3, ACOT11, PCDGHA12, PTGDR, HIST1H4F, GRM4, and SLC22A12) were validated by bisulfite sequencing and eight of these genes (ZNF154, HOXA9, POU4F2, EOMES, CA3, ACOT11, PCDGHA12, PTGDR) were also validated by an independent biological validation involving tumors from new patients wherein the tumors were divided into groups as in the discovery set. Among the 11 genes validated by bisulfate sequencing, the methylation profiles for the tumor markers ZNF154, HOXA9, POU4F2, and EOMES, are shown in FIG. 1A; the remaining 7 genes: ACOT11, PCDHGA12, CA3, PTGDR, HIST1H4F, SLC22A12, and GRM4, methylation profiles are shown in FIG. 2A. The certain genes were identified with significantly (p<0.0001-p<0.05, Mann-Whitney) altered methylation between stages; Stage Ta versus T2-4:697 genes (including CHRNB1, BRF1, and SOCS3 (FIG. 3A)); Stage Ta versus T1:176 genes; stage T1 versus T2-4:137 genes (including SCARF2 (FIG. 3A)), muscle invasive versus non-muscle invasive tumors: 148 genes, and low grade versus high grade tumors: 375 genes. Furthermore, 149 genes were identified as being potential candidate methylation markers of disease progression as they had altered methylation in progressing Ta tumors compared to stable Ta tumors (including the novel marker, TBX4, shown in FIG. 3A).

TABLE 2

| | Discovery set | Validation set | Urine |
|---|---|---|---|
| Characteristics | (Infinium array) | (MS-HRM) | specimens |
| Controls | 6 | 8 | 59 |
| Gender (%) | | | |
| Male | 6 (100) | 8 (100) | 53 (88) |
| Female | 0 | 0 | 7 (12) |
| Age, mean (min-max) | 72 (67-87) | 61 (52-72) | 61 (30-88) |
| Nitrite test | | | |
| Positive (%) | N/A* | N/A | 19 (32) |
| Negative (%) | N/A | N/A | 33 (55) |
| Tumors | 26** | 55 | 115 |
| Gender (%) | | | |
| Male | 18 (69) | 39 (70.9) | 89 (77) |
| Female | 8 (31) | 16 (29.1) | 26 (23) |
| Age, mean (min-max) | 67 (38-87) | 70 (39-89) | 68 (35-93) |
| Ta | 63 (38-80) | 68 (39-87) | 67 (35-93) |
| T1 | 72 (53-83) | 71 (63-78) | 69 (50-79) |
| T2-4 | 78 (69-87) | 72 (56-89) | 68 (45-89) |
| Pathological stage (%) | | | |
| Ta | 17 (65) | 25 (45) | 59 (51) |
| T1 | 5 (19) | 15 (27) | 27 (23) |
| T2 | 4 (15) | 14 (25) | 28 (24) |
| T3 | 0 | 1 (2) | 1 (1) |
| T4 | 0 | 0 | 0 |
| Grade (%) | | | |
| I | 6 (23) | 6 (10.9) | 17 (15) |
| II | 10 (38) | 19 (34.5) | 37 (32) |
| III | 10 (38) | 27 (49.1) | 57 (50) |
| IV | 0 | 2 (3.6) | 4 (3) |
| N/A | 0 | 1 (1.8) | 0 |
| Nitrite test | | | |
| Positive (%) | N/A | N/A | 5 (4) |
| Negative (%) | N/A | N/A | 108 (94) |
| Tumor cells in urine | | | |
| Positive (%) | N/A | N/A | 39 (34) |
| Negative (%) | N/A | N/A | 15 (13) |

*N/A not available
**Additional metachronous tumor information used for intra-patient analyses (suppl. Table 8)

Validation of Microarray Data:

In order to confirm the microarray findings, the MS-HRM technique was also used on an independent sample set consisting of 8 normals and 55 cancers as indicated in Table 2 above showing the demographic and clinical characteristics of the bladder cancer patients and control individuals.

Technical Validation of the MS-HRM Technique:

To test PCR based MS-HRM, a technical validation was performed prior to independent validation. MS-HRM primers for eight bladder cancer marker genes (selected as per FIG. 1B) were tested on 12 clinical samples (two normal and ten tumor samples), which were also included on the Infinium Array. The Pearson correlation coefficient between the Infinium Array and the MS-HRM ranged from 0.75 to 0.99, which was acceptable and served as confirmation.

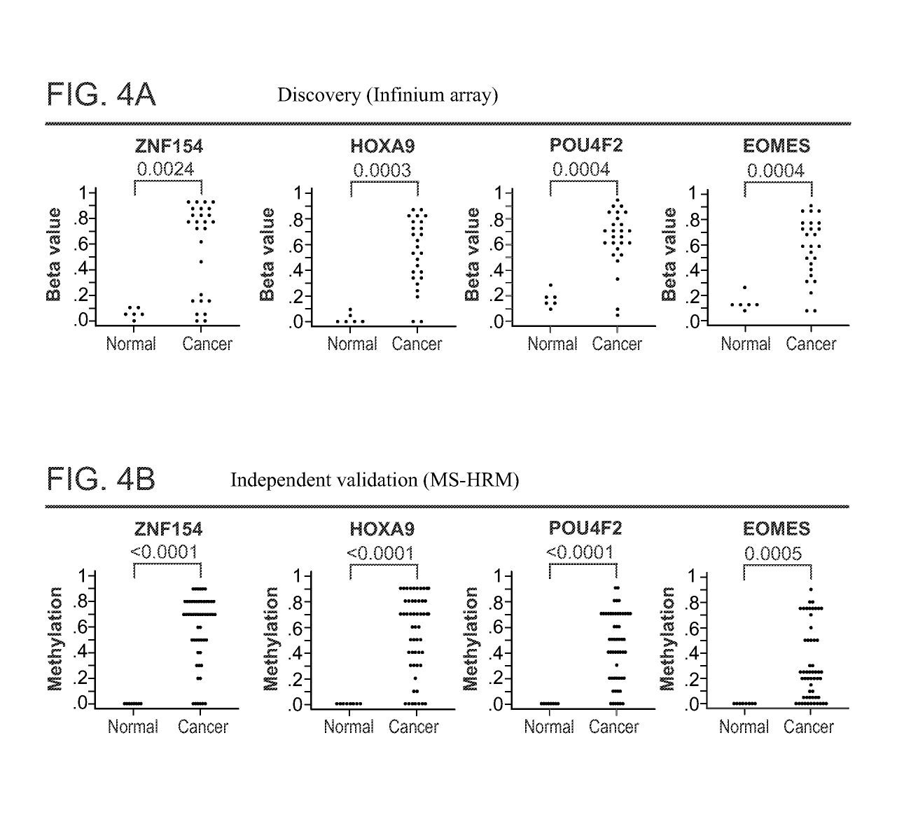

All eight tumor markers ZNF154, HOXA9, POU4F2, EOMES, ACOT11, PCDHGA12, CA3 and PTGDR) were validated by the independent validation set (p<0.011) (see FIG. 4B, FIG. 2B, Supplementary Table 2). In addition to the tumor markers, markers of stage, invasiveness, and candidate markers of tumor progression were also validated (see FIG. 3B and Supplementary Table 3). Most of the stage, invasiveness and progression markers were validated in the independent validation set consisting of 55 tumor samples and 8 normal samples.

The inter observer agreement (Kappa-value) of the MS-HRM validation assay indicated its effectiveness (0.58 to 1.00, see Supplementary Tables 2 and 3). None of the markers identified were independent of each other (see Supplementary Table 4). This indicates that one single methylation mechanism may account for the majority of the methylation alterations discovered.

Bisulfite Sequencing of DNA Surrounding Infinium Probes:

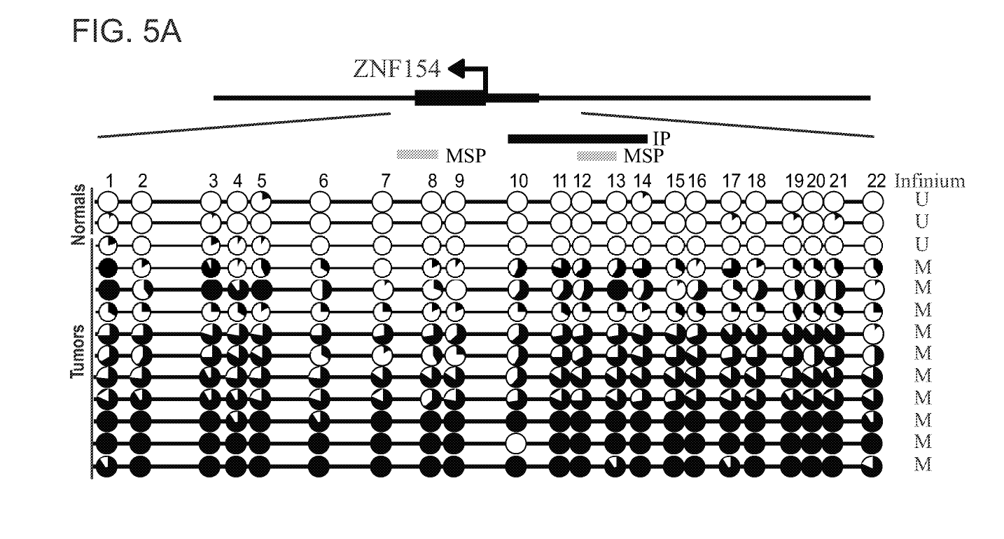

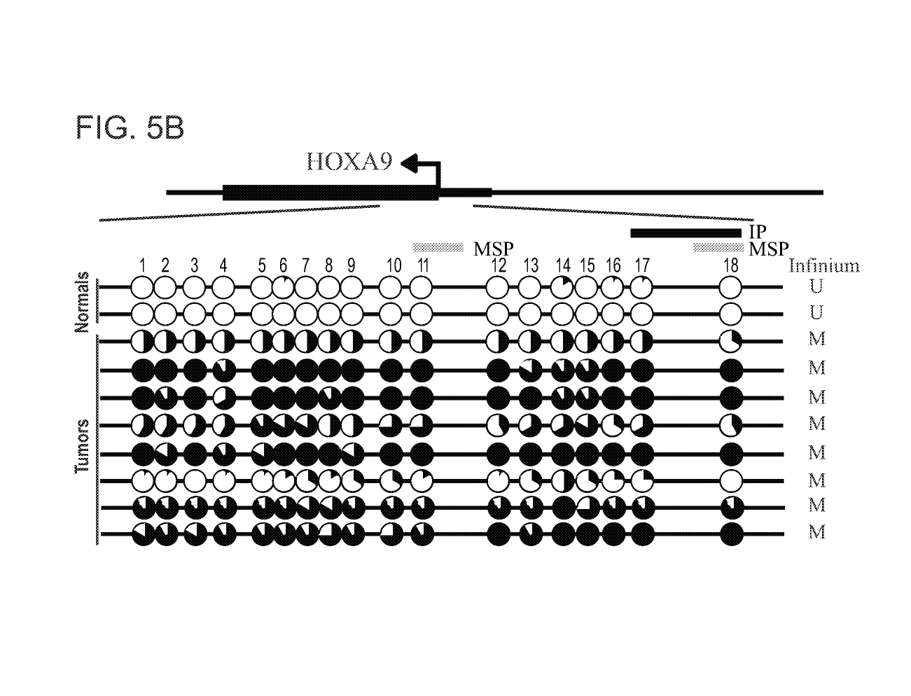

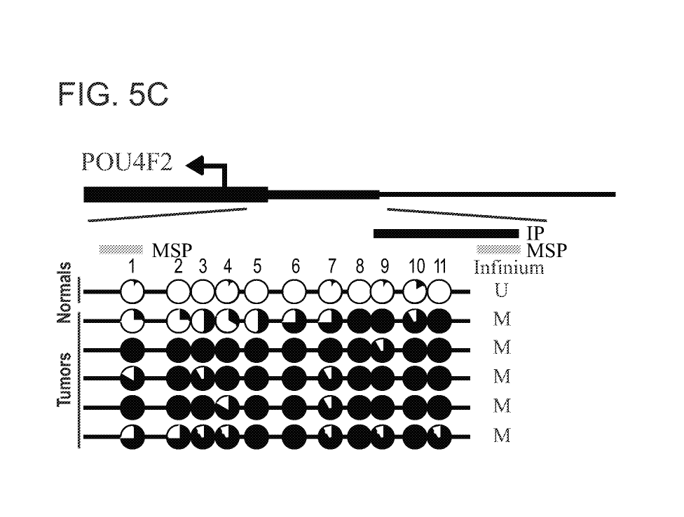

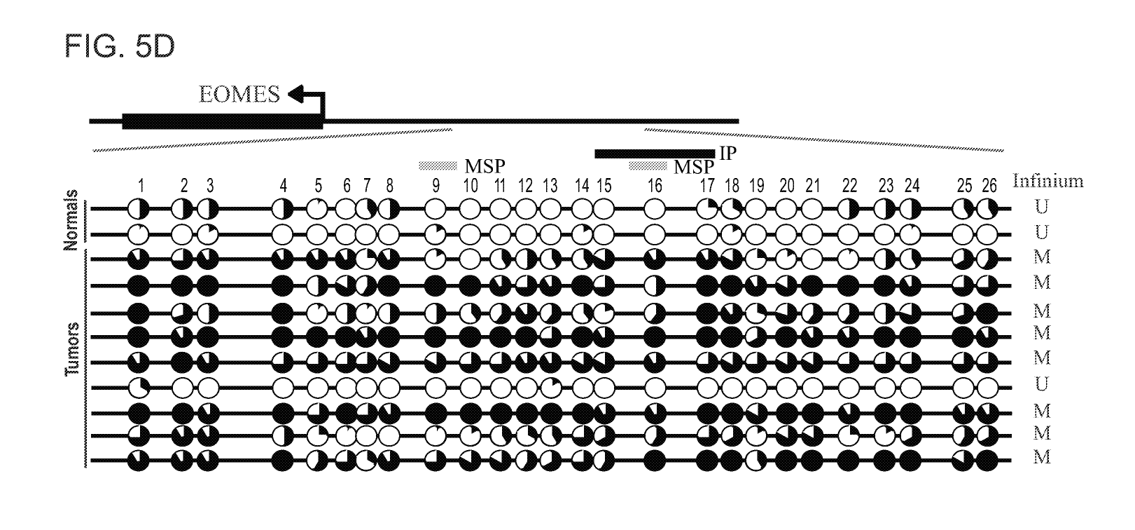

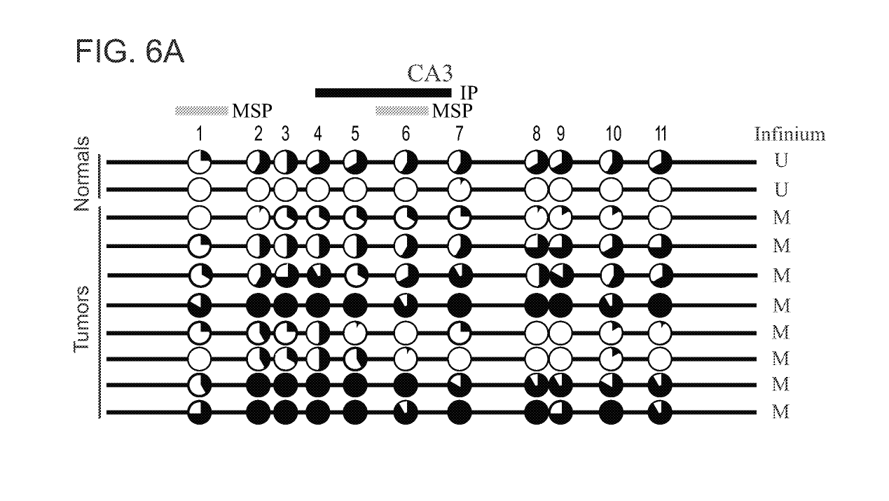

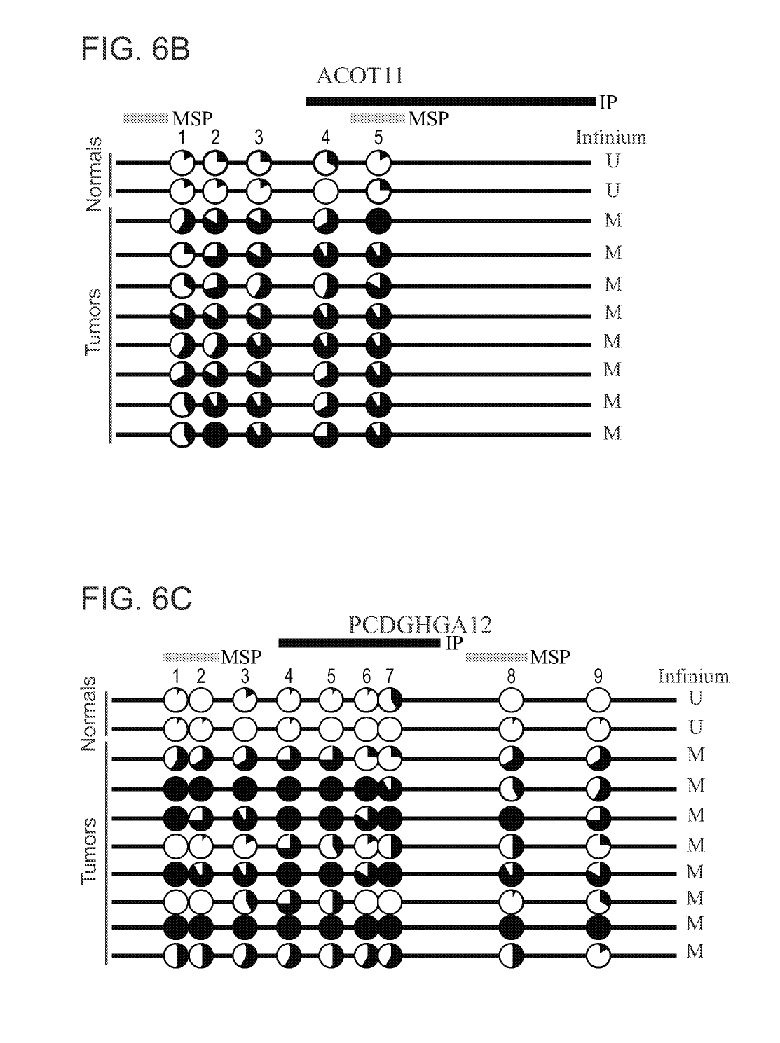

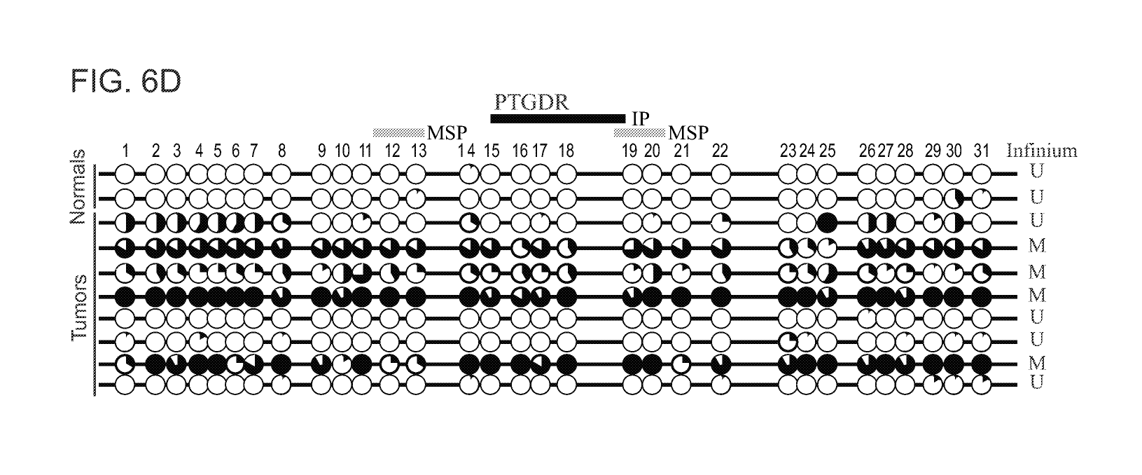

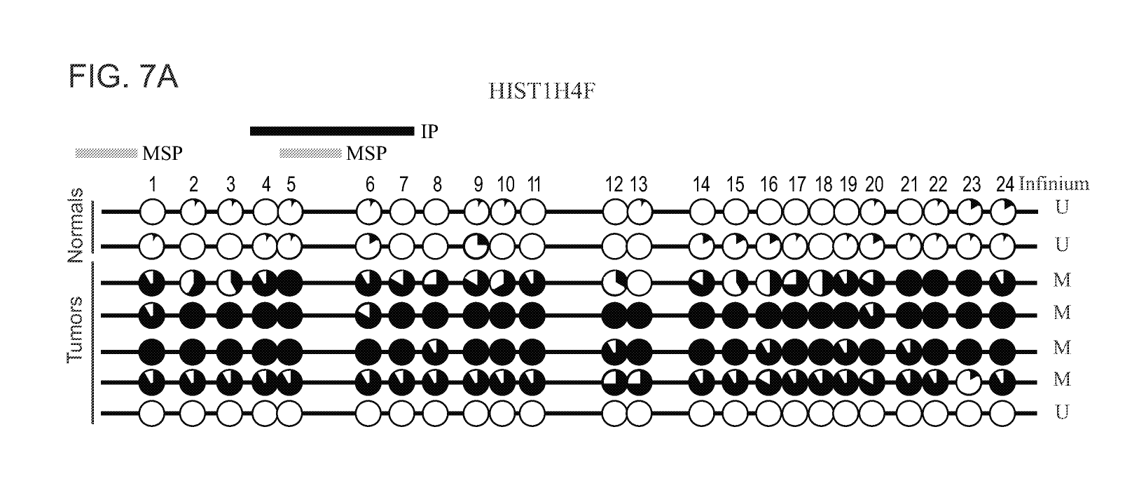

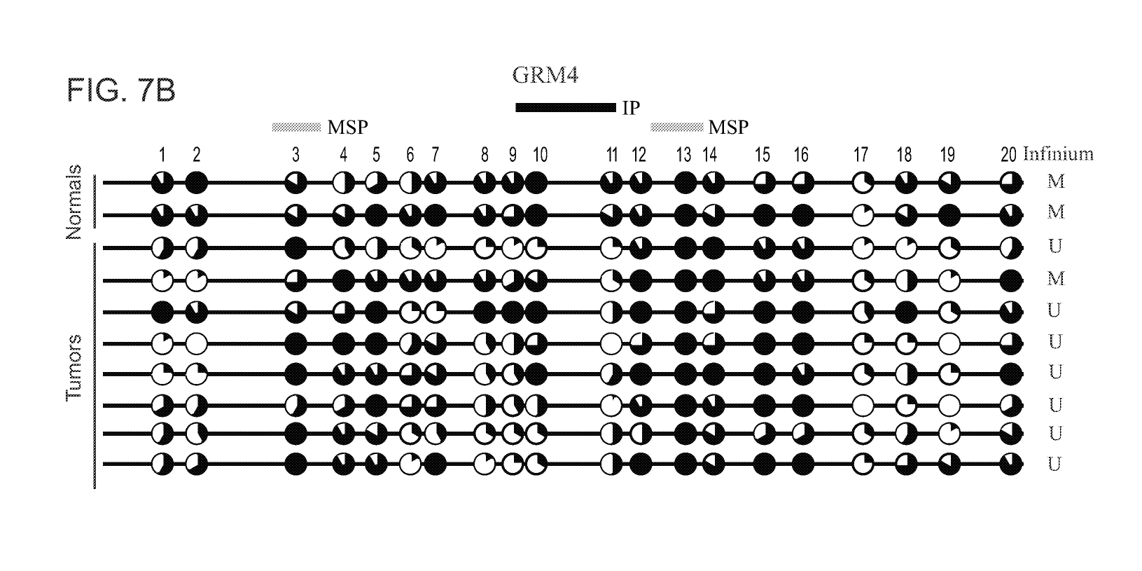

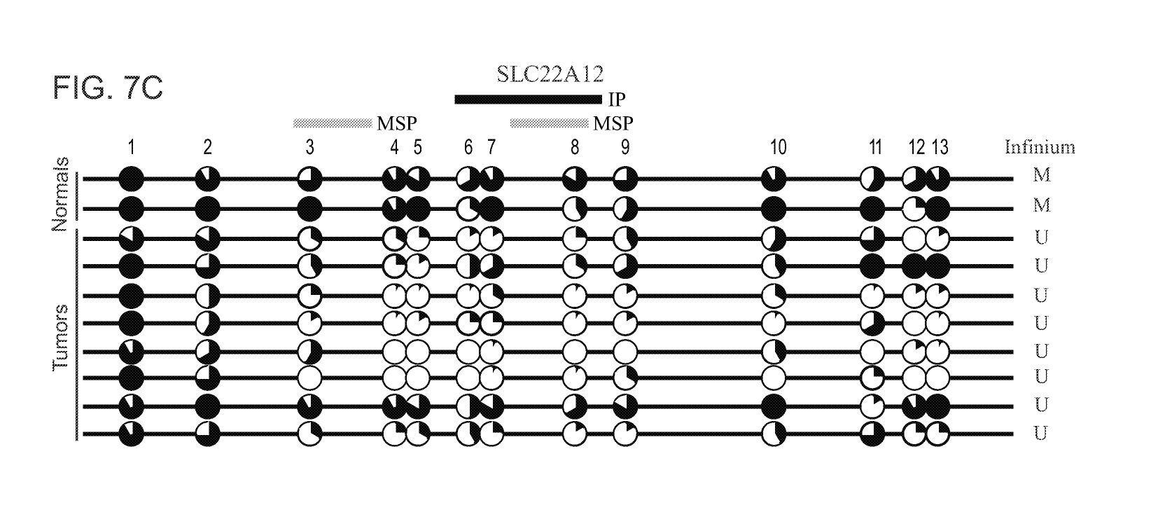

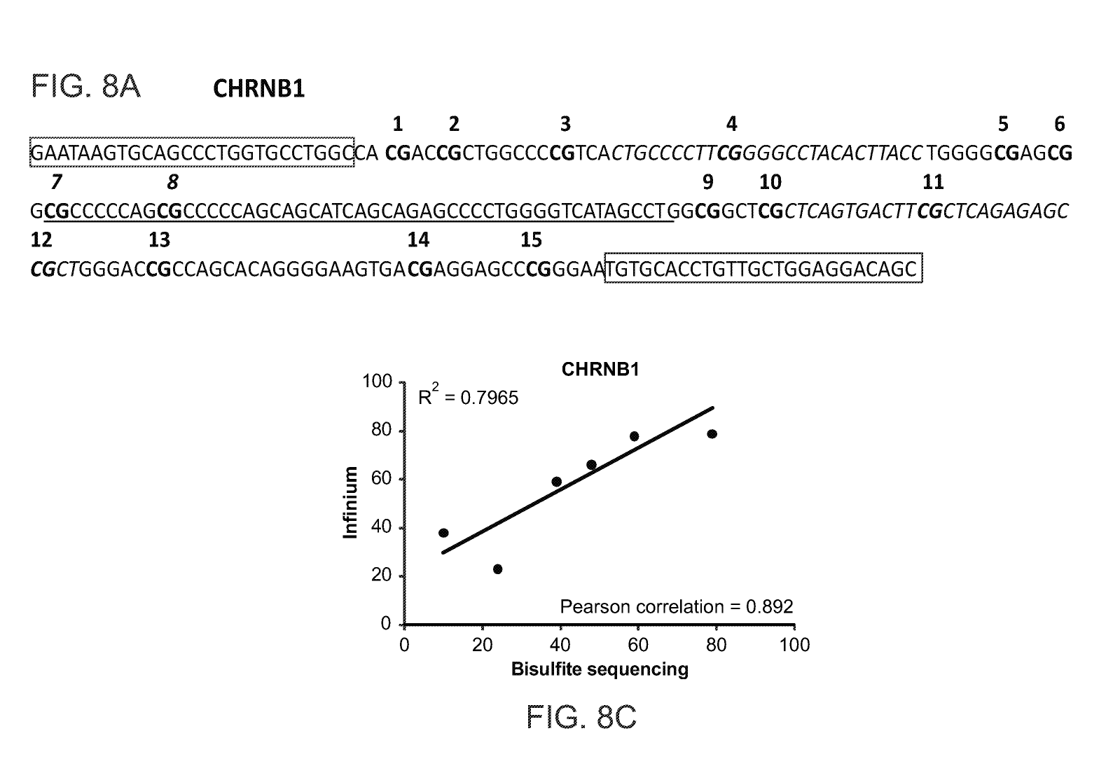

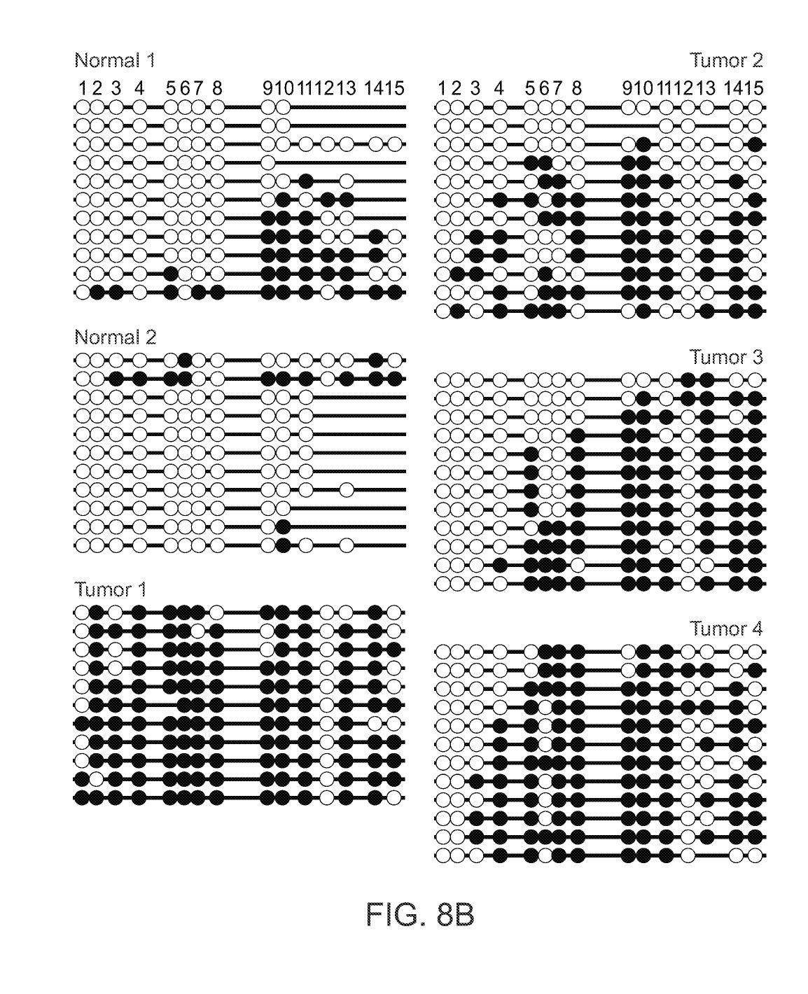

Eleven tumor marker genes and one stage marker were selected for analytical validation by bisulfite sequencing to obtain detailed information on the sequence surrounding the Infinium Array probe source sequence, and the sequence analyzed by MS-HRM. Bisulfite sequencing corresponded well with the array and MS-HRM based findings (See FIG. 5, FIG. 6, FIG. 7, and FIG. 8).

Association Between Methylation Status and Clinicopathological Variables in the Validation Set:

The possible association of methylation status with two clinicopathological parameters, stage and grade, were investigated. Table 3 below indicates association between methylation markers and stage, grade and age in the validation set. Methylation values were dichotomized as positive or negative based on Receiver Operating Characteristic (ROC) analysis. The frequency of methylation is shown, as well as the number of methylation positive tumors and the total number of tumors.

TABLE 3

| | | ZNF154 | HOXA9 | POU4F2 | EOMES | CA3 | PCDHGA12 | ACOT11 | PTGDR |
|---|---|---|---|---|---|---|---|---|---|
| Stage | pTa | 84% (21/24) | 83% (19/23) | 92% (23/25) | 68% (17/25) | 92% (22/24) | 92% (23/25) | 79% (19/24) | 44% (11/25) |
| | pT1 | 100% (15/15) | 100% (15/15) | 100% (15/15) | 93% (14/15) | 100% (15/15) | 93% (14/15) | 100% (15/15) | 80% (12/15) |
| | pT2-4 | 100% (15/15) | 87% (13/15) | 100% (15/15) | 87% (13/15) | 100% (15/15) | 100% (15/15) | 100% (15/15) | 67% (10/15) |
| | P- | 0.184 | 0.303 | 0.495 | 0.153 | 0.497 | 0.786 | 0.049 | 0.079 |
| | Value* | | | | | | | | |
| Grade | I | 67% (4/6) | 100% (6/6) | 100% (6/6) | 50% (3/6) | 67% (4/6) | 83% (5/6) | 100% 6/6) | 33% (2/6) |
| | II | 95% (18/19) | 88% (15/17) | 95% (18/19) | 79% (15/19) | 100% (18/18) | 95% (18/19) | 78% (14/18) | 58% (11/19) |
| | III-IV | 97% (28/29) | 90% (26/29) | 97% (28/29) | 86% (25/29) | 100% (29/29) | 97% (28/29) | 97% (28/29) | 69% (20/29) |
| | P-value | 0.087 | 1.000 | 1.000 | 0.165 | 0.011 | 0.342 | 0.095 | 0.243 |

*Fisher's exact test

Only methylation of ACOT11 was associated with stage (Fisher's exact test, p=0.049). ACOT11 was more frequently methylated in the T1 and T2-4 stage tumors than in the superficial Ta tumors. Another marker CA3 was less frequently methylated in grade I tumors compared to grade II and III tumors (Fisher's exact test, p=0.011). When the tumor patients were divided into two groups by mean age (72) of the patients, no significant association with age was found. However, higher stage was associated with increasing age (Fisher's exact test, p=0.041).

Identification of Methylated Biomarkers in Urinary Specimens from Bladder Cancer Patients:

To test the potential of the validated tumor specific methylation of the genes ZNF154, POU4F2, HOXA9, and EOMES as urinary markers for early detection of bladder cancer, urine from 115 patients with cancer and 59 control urine samples was analyzed using MS-HRM. The results are set forth in Table 4 below.

TABLE 4

| | Sensitivity, % | Specificity, % | | | | | Kappa- |
|---|---|---|---|---|---|---|---|
| Gene | (pos./total*) | (neg./total*) | AUC (95 CI) | PPV % | NPV % | P-value^{#} | value |
| ZNF154 | 62 (68/110) | 100 (57/57) | 0.84 (0.79-0.89) | 100 | 58 | <0.0001 | 0.94 |
| POU4F2 | 66 (75/113) | 100 (54/54) | 0.88 (0.84-0.93) | 100 | 59 | <0.0001 | 0.89 |
| HOXA9 | 74 (79/107) | 96 (46/48) | 0.84 (0.78-0.90) | 98 | 63 | <0.0001 | 0.95 |
| EOMES | 68 (69/101) | 100 (40/40) | 0.89 (0.85-0.93) | 100 | 56 | <0.0001 | 0.89 |
| Combined | 84 (94/112) | 96 (50/52) | 0.90 (0.86-0.94) | 98 | 74 | <0.0001 | N/A^{$} |

*Some urines provided small amount of DNA, not sufficient for all analysis.
^{#}Mann-Whitney U test
^{$}Not applicable

The methylation difference between urine from healthy individuals and patients was highly significant for ZNF154 (p<0.0001), POU4F2 (p<0.0001), HOXA9 (p<0.0044), and EOMES (p<0.0001). The sensitivity observed for the individual markers was 62%-74%, while the specificity was 100% for ZNF154, POU4F2, and EOMES, and 96% for HOXA9 using cut-off values decided by receiver operating characteristic (ROC) analysis. Combining all four markers increased the sensitivity to 82% and the specificity to 97%; with positive predictive value (PPV) of 98% and negative predictive value (NPV) of 73%.

Given that cytology has less sensitivity in low stage lesions, the combined markers were analyzed on urine from 59 patients with Ta tumors. The sensitivity was 80% and specificity 97%, the AUC (95% CI) 0.88 (0.82-0.94), the PPV 96% and the NPV 83% (see Supplementary Table 5). The sensitivity in urine from patients with T1 and T2-4 tumors was slightly higher than for Ta tumors. Cytology also has less sensitivity in low grade tumors. The performance of the combined markers on urine from patients with grade one tumors was: sensitivity 71%, specificity 97%, AUC (95% CI) 0.84 (0.72-0.95), PPV 86%, and NPV 92% (see Supplementary Table 5). The sensitivity on urine specimens with tumor cells detected by the pathologist was 95%, while it was 87% in urine in which the pathologist did not detect cells. Methylation markers were able to detect cancer in 13 of 15 patients where a pathologist did not detect tumor cells in urine samples. Based on this, the urinary methylation assay is much more sensitive than urine cytology for the detection of bladder tumors. Methylation data from urine specimens and tumor samples were matched for 33 patients. The analytical sensitivity of the methylation data on these patient samples ranged from 81%-97%, with a combination of the four methylation markers ZNF154, HOXA9, POU4F2, and EOMES achieving 94% analytical sensitivity (see Supplementary Table 6).

Association Between Methylation Status and Clinicopathological Variables on Urine Specimens:

The association of the four urinary markers of bladder cancer ZNF154, HOXA9, POU4F2, and EOMES with stage, grade, age, cytology, and nitrite status was analyzed (see Supplementary Table 7). Methylation of ZNF154 was associated with higher stage (Fisher's exact test, p=0.019) and grade (Fisher's exact test, p=0.002), whereas methylation of EOMES was associated with high grade (Fisher's exact test, p=0.036). The frequency of methylation of HOXA9 and EOMES was independent of cytology being positive or negative for tumor cells (Fisher's exact test, p>0.05). No association was observed between the frequency of methylation and age for any of the markers (Fisher's exact test, p>0.05). Nitrite positivity did not influence the methylation assay in tumor urine samples nor in normal control urine samples.

Correlation Between DNA Methylation and Transcription:

Considering the genes in Table 1, only HOXA9 and ZNF154 had an absolute Pearson correlation between methylation and expression equal to or larger than 0.4, and only HOXA9 was differentially expressed between normal and tumor samples (p=0.0022, student's t-test). As expected, the level of HOXA9 transcript was lower in tumor compared to normal samples. The bisulfite sequencing did not provide additional information—as the array probes seemed to reflect the methylation event well in the sequenced areas (see FIGS. 5, 6 and 7).

Intrapatient Variation in Methylation:

The intrapatient stability of methylation was high for both Ta(stable) and Ta(prog) tumors, as 92% and 89% of changes, respectively, found in early tumors were present later on. The number of changes was independent of time between tumors (R²=0.0029) and mRNA transcript level of DNA-methyltransferases. However, to study if this was based on a systematic change in methylation of certain genes over time; a group comparison was made across the metachronous samples. This analysis revealed that no single genes were differentially methylated between the first and second tumor within the stable or progressing groups (p>0.05; Wilcoxon signed-rank test).

Pathway Analysis of Differentially Methylated Genes:

Using Gene Ontology (GO), the 149 differentially methylated genes between Ta stable and Ta progressing tumors belonged mainly to 22 overrepresented pathways, having up to 7 methylation changes. Hypermethylated pathways were related to cellular development, in particular, epidermal development (p<0.037). Hypomethylated pathways were related to cell-cell signaling, in particular negative regulators of cell death (p<0.038). Using Ingenuity pathway analysis (IPA), the main network associated functions altered by methylation were cell movement of eukaryotic cells (p=1.65E-010), tumorigenesis (p=3.37E-08), and growth of cancer cells (p=4.46E-07) (see Supplementary Table 8) as well as apoptosis (p<1.24E-06) and proliferation of cells (p<3.91E-06). The top canonical pathway was G-protein coupled receptor signaling (p=9.96E-06 to p=1.56E-02, see Supplementary Table 8).

Pathway analysis on superficial papillomas of low histological grade versus high grade superficial and invasive tumors, showed that many of the top networks identified between Ta stable and Ta progressing tumors were also present in this analysis (see Supplementary Table 8). These results suggest that methylation may hit selected networks and pathways at multiple levels, thereby impacting the malignant process.

Epigenetic Regulation of Keratin (KRT), Keratin Associated Proteins (KRTAP), and Small Proline Rich Proteins (SPRR):

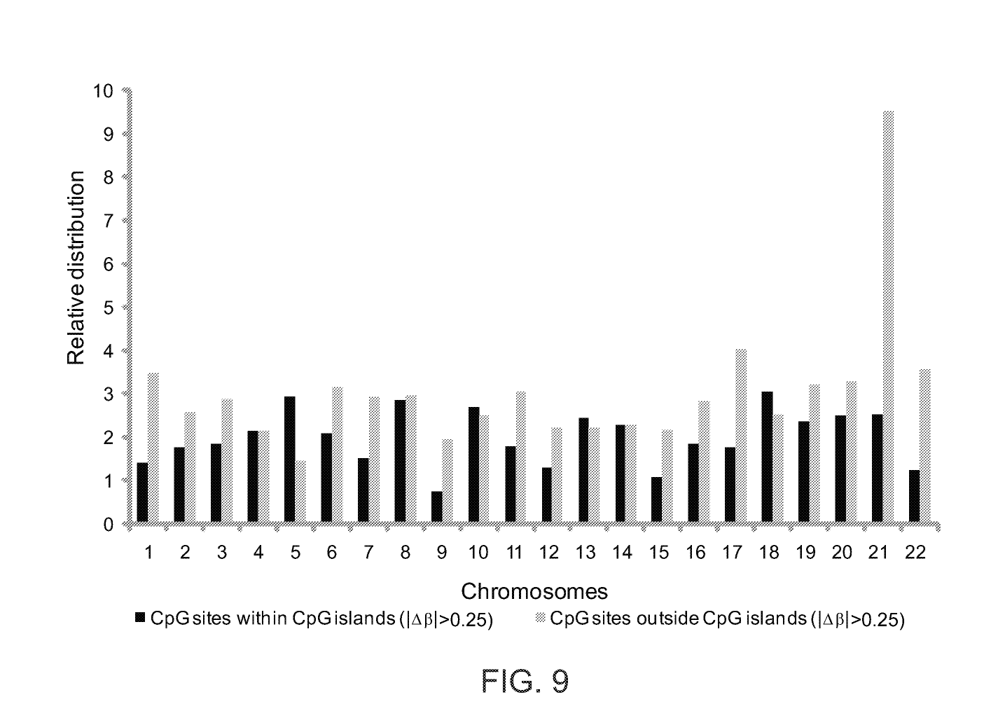

Chromosome 21 was found to encompass more differentially methylated genes outside CpG islands, than any other chromosome after correction for number of CpG sites (p<0.0001) (see FIG. 9). Chromosome 21 furthermore contains many genes encoding keratin-associated proteins (KRTAP). In 16 of these, hypomethylation was detected (Δβ<−0.25 and p<0.0001 to p=0.019), and three of the genes (KRTAP13-1, KRTAP19-2, and KRTAP20-2) had significantly (p<0.05) increased transcript expression. A set of keratin related genes have previously been shown to be upregulated in bladder cancer and associated with squamous cell metaplasias. See Dyrskjot L, et al. Identifying distinct classes of bladder carcinoma using microarrays, Nat Genet 2003; 33:90-96). Analysis of this set showed the small proline rich proteins SPRR1A/2D/3 on chromosome 1 to be hypomethylated in cancer and SPRR3 expression to be up-regulated (p<0.0001). Of the neutral keratins located on chromosome 12, five showed hypomethylation KRT2A/6B/6C/7/8, (Δβ<−0.25 and p=0.0001 to p=0.0022) and KRT6B/7/8 showed increased expression (p<0.05). The acidic keratins on chromosome 17 showed hypomethylation of KRT10/19/20 and up-regulated expression of KRT20 (p=0.0027). The Pearson correlations between methylation and expression were −0.84, −0.50, −0.66, and −0.91 for KRT7/8/19/20, respectively. Thus, the keratins and keratin related proteins seem to be epigenetically regulated in bladder cancer.

### Example II

Patient Material:

A total of 652 voided urine samples were collected at the Department of Urology at Aarhus University Hospital from 390 bladder cancer patients, and 47 individuals with benign prostatic hyperplasia or bladder stones, but no history of bladder cancer (control individuals). From these, 227 samples were excluded, as the DNA amount was below a set threshold. See Table 5 below.

TABLE 5

| Characteristics | Control individuals |
|---|---|
| Individual with no history | 12 |
| of BC | |
| Gender, n (%) | |
| Male | 11 (92) |
| Female | 1 (8) |
| Age, mean (min-max) | 64 (51-80) |
| Nitrite test, n (%) | |
| Positive | 1 (8) |
| Negative | 11 (92) |

| | | Patients with | Patients without |
|---|---|---|---|
| | All patients - | recurrent tumor | tumor at control |
| Characteristics | first visit | at control visit | visit |
| Bladder cancer | 80 | 63 | 49 |
| patients | | | |
| Samples collected | 80 | 75 | 60 |
| Primary cases | 21 | | |
| Recurrent cases | 59 | 75 | |
| Gender, n (%) | | | |
| Male | 61 (76) | 59 (79) | 55 (92) |
| Female | 19 (24) | 16 (21) | 5 (8) |
| Age, mean (min-max) | 67 (33-83) | 70 (34-85) | 68 (43-84) |
| Ta | 67 (33-83) | 70 (34-85) | |
| T1 | 69 (56-80) | 73 (65-82) | |
| CIS | 67 (67-67) | 68 (57-73) | |
| T2-4 | 0 | 72 (69-74) | |
| Pathological stage, | | | |
| n (%) | | | |
| Ta | 66 (83) | 57 (76) | |
| T1 | 13 (16) | 11 (15) | |
| CIS | 1 (1) | 5 (7) | |
| T2-4 | 0 | 2 (3) | |
| Grade, n (%)^{a} | | | |
| I | 16 (20) | 14 (19) | |
| II | 36 (45) | 34 (45) | |
| III | 28 (35) | 27 (36) | |
| Nitrite test, n (%) | | | |
| Positive | 3 (4) | 4 (5) | 2 (3) |
| Negative | 75 (94) | 66 (88) | 57 (95) |
| N/A^{b} | 2 (3) | 5 (7) | 1 (2) |
| Tumor cells in urine, | | | |
| n (%) | | | |
| Positive | 41 (51) | 31 (41) | 18 (30) |
| Negative | 33 (41) | 32 (43) | 27 (45) |
| N/A | 6 (8) | 12 (16) | 15 (25) |

^{a}Bergkvist
^{b}Not available

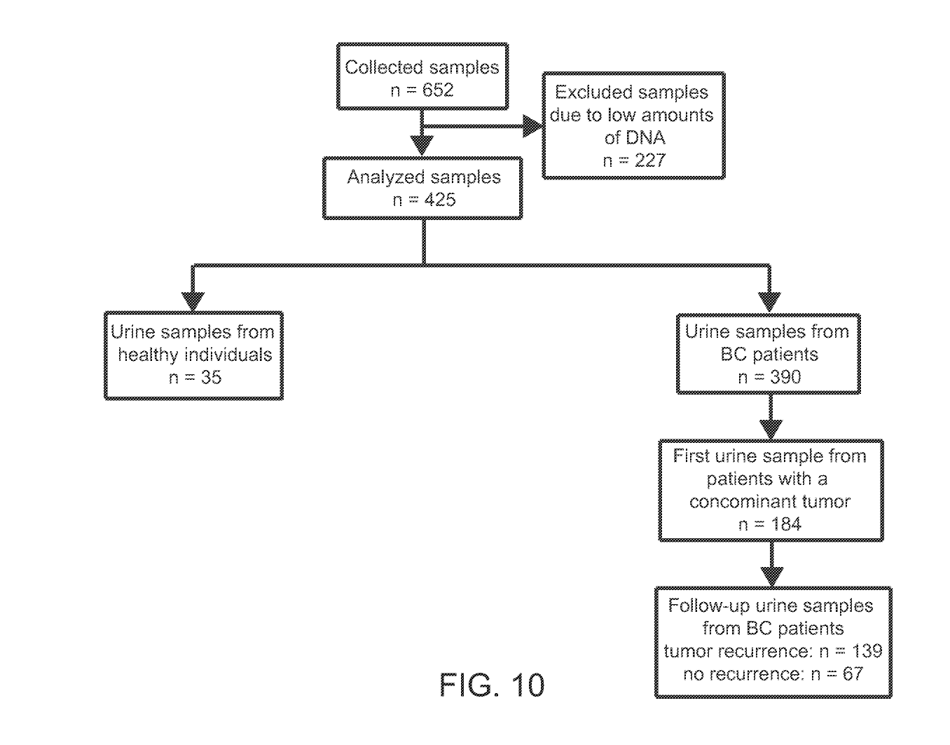

The remaining 425 samples (390 samples from 184 BC patients and 35 from control individuals) are indicated in Table 6 below and FIG. 10.

TABLE 6

| Characteristics | Control individuals |
|---|---|
| Individuals with no | 35 |
| history of BC (controls) | |
| Gender, n (%) | |
| Male | 30 (86) |
| Female | 5 (14) |
| Age, mean (min-max) | 70 (35-88) |
| Nitrite test, n (%) | |
| Positive | 15 (43) |
| Negative | 20 (57) |

| | | Patients with | Patients without |
|---|---|---|---|
| | All patients - | recurrent tumor | tumor at |
| Characteristics | first visit | at control visit | control visit |
| Bladder cancer | 184 | 101^{c} | 57^{c} |
| patients | | | |
| Samples collected | 184 | 139 | 67 |
| Primary cases | 44 | | |
| Recurrent cases | 140 | 139 | |
| Gender, n (%) | | | |
| Male | 148 (81) | 106 (76) | 58 (87) |
| Female | 36 (19) | 33 (24) | 9 (13) |
| Age, mean (min-max) | 69 (33-89) | 71 (43-89) | 69 (49-86) |
| Ta | 69 (33-85) | 70 (43-87) | |
| T1 | 70 (42-89) | 74 (43-89) | |
| CIS | 71 (67-74) | 73 (66-81) | |
| T2-4 | 0 | 71 (43-83) | |
| Pathological stage, | | | |
| n (%) | | | |
| Ta | 132 (72) | 92 (66) | |
| T1 | 50 (27) | 29 (21) | |
| CIS | 2 (1) | 5 (4) | |
| T2-4 | 0 | 13 (9) | |
| Grade, n (%)^{b} | | | |
| I | 17 (9) | 12 (9) | |
| II | 74 (40) | 55 (40) | |
| III | 93 (51) | 71 (51) | |
| Nitrite test, n (%) | | | |
| Positive | 16 (9) | 13 (9) | 7 (10) |
| Negative | 163 (89) | 121 (87) | 57 (85) |
| N/A^{a} | 5 (3) | 5 (4) | 3 (5) |
| Tumor cells in urine, | | | |
| n (%) | | | |
| Positive | 119 (65) | 87 (63) | 22 (33) |
| Negative | 28 (15) | 25 (18) | 24 (36) |
| N/A | 37 (20) | 27 (19) | 21 (31) |

^{a}N/A Not available.
^{b}Bergkvist.
^{c}Of the 184 patients, 26 were lost for follow-up.

Ten to fifty milliliters (mL) of urine was collected at regular follow-up visits. Urine specimens were collected immediately before cystoscopy; cells were sedimented by centrifugation, and frozen at −80° C. The tumors were staged according to the TNM system described in Sobin, TNM Classification of malignant Tumours, International Union Against Cancer, 2002, 6^{th} Edition (New York, N.Y.: John Wiley & Sons and graded according to Bergkvist et al., Classification of bladder tumours based on the cellular pattern. Preliminary report of a clinical-pathological study of 300 cases with a minimum follow-up of eight years, Acta Chir Scand, 1965, 130(4): p. 371-78. Fifteen of the control individuals were stix positive for nitrite in the urine indicating bacterial infection. Informed written consent was obtained from all patients, and research protocols were approved by The Central Denmark Region Committees on Biomedical Research Ethics. Patient treatment and follow-up were performed in accordance with the guidelines of European Association of Urology as set forth in Babjuk et al., EAU Guidelines on non-muscle-invasive urothelial carcinoma of the bladder, Eur Urol, 2008, 54(2):p. 303-14.

DNA Extraction and Bisulfite Modification:

DNA was extracted with the QIAsymphony Virus/Bacteria Midi kit (96) (Qiagen) using the QIAsymphony® SP instrument and employing the Complex800_V5_DSP protocol. Five hundred nanograms (500 ng) of DNA was bisulfite modified using the EZ-96 DNA methylation D5004 kit (Zymo Research) according to the manufacturers recommendations and eluted in 60 microliters (μl) of elution buffer and stored at −20° C. until use.

Real-Time Quantitative Methylation-Specific Polymerase Chain Reaction (MethyLight):

Methylation analysis was performed using MethyLight as described in Campan et al., MethyLight, Methods Mol Biol, 2009, 507: p. 325-37. Primers and probes for the six genes of interest were designed to include eight to ten CpG dinucleotides as indicated in Table 7 below. All probes contain a 6-FAM fluorophore at the 5′ end and a black hole quencher-1 (BHQ-1) at the 3′ end.

TABLE 7

| Gene | Sense primer (5' to 3') | Antisense primer (5' to 3') | Probe (5' to 3') | Amplification protocols |
|---|---|---|---|---|
| ALU-C4 | GGTTAGGTATAGTGGTTTATATT | ATTAACTAAACTAATCTTAAACTCCTA | CCTACCTTAACCTCCC | 95° C. 10 [symbol] , (95° C. 15 [symbol] , |
| | TGTAATTTTAGTA | ACCTCA | | 60° C. 1 [symbol] ) × 45 |
| ZNF154 | TTTATCGGATTAGAGATAGTAGA | TAACGTAAATCCCCCAAAACGACG | AACGACGACTCCCCTC | 95° C. 10 [symbol] , (95° C. 15 [symbol] , |
| | GCGT | | ACGCCTT | 60° C. 1 [symbol] ) × 45 |
| POU4F2 | GTTGTGCGAAGTTGAGTTTATTC | CCGTTCAAACTAACAACAAAAACGA | CGGATTTTGTACGTTT | 95° C. 10 [symbol] , (95° C. 15 [symbol] , |
| | | | GATTTCGGTTAC | 60° C. 1 [symbol] ) × 45 |
| HOXA9 | GTGGTTATTATCGTGTTTAGCGT | CCGATACCACCAAATTATTACATA | TGGTTCGTTCGGTTCG | 95° C. 10 [symbol] , (95° C. 15 [symbol] , |
| | | | ATTTACGGA | 60° C. 1 [symbol] ) × 45 |
| EOMES | GGTTGGGGAAGTAGAGTTTCGAT | ATAAACAATTACAAACGCCGCCA | CGCTCCGAAAACGCAT | 95° C. 10 [symbol] , (95° C. 15 [symbol] , |
| | | | TTTCCGACTA | 60° C. 1 [symbol] ) × 45 |
| TWIST1 | GTTAGGGTTCGGGGGCGTTGTT | CCGTCGCCTTCCTCCGACGAA | CGGCGGGGAAGGAAAT | 95° C. 10 [symbol] , (95° C. 15 [symbol] , |
| | | | CGTTTC | 60° C. 1 [symbol] ) × 45 |
| VIM | TTCGGGAGTTAGTTCGCGTT | ACCGCCGAACATCCTACGA | TCGTCGTTTAGGTTAT | 95° C. 10 [symbol] , (95° C. 15 [symbol] , |
| | | | CGT | 60° C. 1 [symbol] ) × 45 |

[symbol]

indicates data missing or illegible when filed

For normalization of DNA input material, the ALU-C4 repeat element sequence was used as described in Weisenberger et al., Analysis of repetitive element DNA methylation by MethyLight, Nucleic Acids Res, 2005, 33(21): p. 6823-36. Quantitative PCR amplifications were carried out with the TaqMan Universal PCR Master Mix No AmpErase (Applied Biosystems) according to the manufacturer's instructions in duplicates using 2 μl (5 ng) of bisulfite modified DNA in a final volume of 5 μl in 384-well plates on a ABI 7900 HT Fast Real Time PCR System (Applied Biosystems). When inconsistency between duplicates occurred, the analysis was repeated. Amplification protocols for real-time quantitative methylation-specific polymerase chain reactions were used. Amplification data was analyzed by the sequence detector system (SDS 2.4, Applied Biosystems). Each plate included a serial dilution (25-0.04 ng) of fully methylated DNA: CpGenome™ Universal Methylated DNA) (Millipore) with the gene of interest and ALU-C4, several no template control (NTC) wells, five nanograms (5 ng) of a methylated control sample: CpGenome™ Universal Methylated DNA (Millipore), and five nanograms (5 ng) unmethylated sample consisting of whole genome amplified DNA from peripheral blood DNA. The percentage of methylated reference (PMR) was calculated for each sample according to the equation: 100×[(gene-x copy value)ₛₐₘₚₗₑ/(ALU-C4 copy value)ₛₐₘₚₗₑ][(gene-x copy value)_{Universal Methylated DNA} (ALU-C4copy value)_{Universal Methylated DNA}]. To classify each sample as methylated or unmethylated, a cutoff value was defined on the basis of mean+2× standard deviation of the methylation levels in urine samples from control individuals (including only those samples having methylation values above zero). PMR values used to define hypermethylation for each marker were: PMR (ZNF154)≧1.51, PMR (EOMES)≧0.348, PMR (HOXA9)≧0.077, PMR (POU4F2)≧0.371, PMR (TWIST1)≧0.405, and PMR (VIM)≧0.368.

Statistical Analysis:

Stata 11 (Statacorp, Tex., USA) was used for all statistical calculations. Two tailed tests were considered statistically significant if P<0.05. Methylation differences were evaluated by nonparametric Wilcoxon-Mann-Whitney test. Fisher's exact test was used for analyzing dichotomous variables. The exact χ²test was used for analyzing associations between clinico-pathological parameters with two or more categories. Correlations of the methylation levels of the markers were calculated with Spearman correlation coefficients. A ROC curve was prepared for each marker and combinations of markers by plotting sensitivity against (1-specificity) and the area under the curve (AUC) was calculated. Log-Rank tests were applied to evaluate equality of survival and Kaplan-Meier survival plots were used for visualization. Multivariate Cox regression analysis was used to analyze covariation between methylation markers, stage, grade, tumor multiplicity, and CIS.

Results:

The analysis of urine data was separated into two parts. The first available urine from each patient was analyzed and compared to non-malignant control urine samples. Then, the methylation markers in urine samples taken during follow-up of each patient were analyzed. The first available urine was from the incident tumor visit in 44 out of 184 cases, and from later recurrences in 140 cases. The level of the six markers: ZNF154, EOMES, HOXA9, POU4F2 (see Reinert et al., Clin Cancer Res, 2011), TWIST1 (see Renard et al., Eur Urol, 2009), and VIM (see Costa et al., Clin Cancer Res, 2010, 16(23): p. 5842-51) in the first available urine was compared to urine from 35 controls as indicated in Table 6. All six markers were highly significantly hyper-methylated in the urine from bladder tumor patients compared to controls, when analyzing both incident and recurrent tumors (Mann-Whitney, P<0.0001) as indicated in Table 8 and Table 9 below.

TABLE 8

| | Sensitivity, % | Specificity, % | | | | |
|---|---|---|---|---|---|---|
| Gene | (pos./total^{a}) | (neg./total) | AUC (95% CI) | PPV^{b}, % | NPV^{c}, % | P value^{d} |
| ZNF154 | 87 (160/184) | 100 (35/35) | 0.95 (0.93-0.97) | 100 | 59 | <0.0001 |
| EOMES | 88 (160/182) | 97 (34/35) | 0.96 (0.94-0.99) | 99 | 61 | <0.0001 |
| HOXA9 | 82 (141/173) | 100 (35/35) | 0.91 (0.88-0.94) | 100 | 52 | <0.0001 |
| POU4F2 | 85 (154/182) | 94 (33/35) | 0.94 (0.91-0.97) | 99 | 54 | <0.0001 |
| TWIST1 | 88 (159/180) | 100 (35/35) | 0.94 (0.92-0.97) | 100 | 63 | <0.0001 |
| VIM | 89 (159/179) | 100 (35/35) | 0.97 (0.94-0.99) | 100 | 64 | <0.0001 |
| Cytology | 81 (119/147) | N/A^{e} | N/A | 100 | N/A | N/A |

^{a}Some urine samples provided inconclusive results for some markers
^{b}Positive predictive value
^{c}Negative predictive value
^{d}Mann-Whitney U test
^{e}Not available

TABLE 9

| | | | | | | |
|---|---|---|---|---|---|---|
| | Sensitivity, % | Specificity, % | | | | |
| Gene | (pos./total^{a}) | (neg./total) | AUC (95% CI) | PPV^{b}, % | NPV^{c}, % | P value^{d} |

First urine analyzed from an incident tumor visit

| | | | | | | |
|---|---|---|---|---|---|---|
| ZNF154 | 98 (43/44) | 100 (35/35) | 0.99 (0.96-1.00) | 100 | 97 | <0.0001 |
| EOMES | 95 (42/44) | 97 (34/35) | 1.00 (0.99-1.00) | 98 | 94 | <0.0001 |
| HOXA9 | 86 (38/44) | 100 (35/35) | 0.92 (0.87-0.98) | 100 | 85 | <0.0001 |
| POU4F2 | 100 (43/43) | 94 (33/35) | 1.00 (1.00-1.00) | 96 | 100 | <0.0001 |
| TWIST1 | 93 (41/44) | 100 (35/35) | 0.98 (0.95-1.00) | 100 | 92 | <0.0001 |
| VIM | 95 (42/44) | 100 (35/35) | 0.98 (0.95-1.00) | 100 | 95 | <0.0001 |
| Cytology | 87 (33/38) | N/A^{e} | N/A | 100 | N/A | N/A |

First urine analyzed from a recurrent tumor visit

| | | | | | | |
|---|---|---|---|---|---|---|
| ZNF154 | 84 (117/140) | 100 (35/35) | 0.95 (0.93-0.97) | 100 | 60 | <0.0001 |
| EOMES | 86 (118/138) | 97 (34/35) | 0.96 (0.94-0.99) | 99 | 63 | <0.0001 |
| HOXA9 | 80 (103/129) | 100 (35/35) | 0.91 (0.88-0.94) | 100 | 57 | <0.0001 |
| POU4F2 | 80 (111/139) | 94 (33/35) | 0.94 (0.91-0.97) | 98 | 54 | <0.0001 |
| TWIST1 | 87 (118/136) | 100 (35/35) | 0.94 (0.92-0.97) | 100 | 66 | <0.0001 |
| VIM | 87 (117/135) | 100 (35/35) | 0.97 (0.94-0.99) | 100 | 66 | <0.0001 |
| Cytology | 79 (86/109) | N/A | N/A | 100 | N/A | N/A |

^{a}Some urine samples provided inconclusive results for some markers
^{b}Positive predictive value
^{c}Negative predictive value
^{d}Mann-Whitney U test
^{e}Not available

Better sensitivities and specificities of the markers were observed when analyzing urine from incident tumor visits compared to urine from recurrent tumor visits as indicated in Table 9. No association was observed between the individual markers and stage, but ZNF154, EOMES, POU4F2, and VIM were more methylated in grade III lesions compared to grade I lesions (Fisher's exact test, P≦0.048). See Table 10 below.

TABLE 10

| | | ZNF154 | | EOMES | | HOXA9 | | POU4F2 | | TWIST1 | | VIM | |
|---|---|---|---|---|---|---|---|---|---|---|---|---|---|
| Stage | pTa | 84% | (111/132) | 86% | (112/130) | 80% | (99/124) | 83% | (108/130) | 85% | (109/128) | 89% | (113/127) |
| | pT1 | 94% | (47/50) | 92% | (46/50) | 87% | (41/47) | 83% | (44/50) | 96% | (48/50) | 88% | (44/50) |
| | CIS | 100% | (2/2) | 100% | (2/2) | 50% | (1/1) | 100% | (2/2) | 100% | (2/2) | 100% | (2/2) |
| | p-value* | 0.188 | | 0.572 | | 0.229 | | 0.640 | | 0.125 | | 0.841 | |
| Grade | I | 71% | (12/17) | 65% | (11/17) | 80% | (12/15) | 59% | (10/17) | 71% | (10/14) | 75% | (12/16) |
| | II | 77% | (57/74) | 85% | (61/72) | 80% | (56/70) | 82% | (60/73) | 90% | (66/73) | 86% | (60/70) |
| | III | 98% | (91/93) | 95% | (88/93) | 83% | (73/88) | 91% | (84/92) | 89% | (83/93) | 94% | (87/93) |
| | P-value | <0.001 | | 0.002 | | 0.837 | | 0.004 | | 0.132 | | 0.048 | |
| Age, years | ≦70 | 84% | (80/95) | 81% | (76/94) | 77% | (67/87) | 81% | (76/94) | 87% | (81/93) | 86% | (77/90) |
| | >70 | 90% | (80/89) | 95% | (84/88) | 86% | (74/86) | 90% | (78/88) | 90% | (78/87) | 92% | (82/89) |
| | P-value | 0.281 | | 0.003 | | 0.170 | | 0.157 | | 0.648 | | 0.235 | |
| Tumor size, cm | <3 cm | 84% | (125/149) | 85% | (125/147) | 81% | (112/139) | 81% | (119/147) | 86% | (125/145) | 87% | (125/144) |
| | >3 cm | 100% | (23/23) | 100% | (23/23) | 91% | (21/23) | 100% | (23/23) | 100% | (23/23) | 96% | (22/23) |
| | P-value | 0.047 | | 0.047 | | 0.376 | | 0.016 | | 0.079 | | 0.315 | |
| Cytology | Positive | 95% | (113/119) | 96% | (113/118) | 86% | (99/115 | 90% | (106/118) | 91% | (108/119) | 94% | (108/115) |
| | Negative | 54% | (15/28) | 64% | (18/28) | 68% | (17/25) | 64% | (18/28) | 80% | (20/25) | 64% | (18/28) |
| | P-value | <0.001 | | <0.001 | | 0.041 | | 0.002 | | 0.156 | | <0.001 | |
| Stix, nitrite | Positive | 75% | (12/16) | 81% | (13/16) | 93% | (14/15) | 81% | (13/16) | 81% | (13/16) | 80% | (12/15) |
| | Negative | 88% | (144/163) | 88% | (142/161) | 80 | (123/153) | 85% | (137/161) | 89% | (142/159) | 89% | (142/159) |

| | | | | | | |
|---|---|---|---|---|---|---|
| P-value* | 0.131 | 0.426 | 0.309 | 0.715 | 0.400 | 0.386 |

*Fisher's exact test

ZNF154, EOMES, and POU4F2 were less methylated in tumors with a size below 3 cm. (Fisher's exact test, P≦0.047). The methylation level of EOMES was associated with age (Fisher's exact test, P≦0.003). There was no association between any of the markers and nitrite status that indicated bacterial infection. None of the markers identified were independent of each other (Spearman's p test, P<0.0001) (data not shown).

Detection of Recurrences by Methylation Markers:

To test the clinical usefulness of the markers, 206 urine samples from the follow-up of 158 patients, 139 samples from patients with a recurrent bladder tumor, and 67 samples from patients with no tumor recurrence were analyzed. See Table 6. Employing the cut-points determined initially, using the control individuals, and only analyzing samples where the first sample was positive for methylation, sensitivity in the range from 87% to 94% was obtained, and specificity in the range from 28%-47%, AUC (95% CI) ranged from 0.70 (0.61-0.80) to 0.78 (0.71-0.86), PPV ranged from 72%-78%, and NPV from 55%-78% as indicated in Table 11 below.

TABLE 11

| | Sensitivity, % | Specificity, % | | | | |
|---|---|---|---|---|---|---|
| Gene | (pos./total^{a}) | (neg./total^{a}) | AUC (95% CI) | PPV, % | NPV, % | P value^{b} |
| ZNF154 | 93 (115/123) | 47 (29/62) | 0.78 (0.71-0.86) | 78 | 78 | <0.0001 |
| EOMES | 94 (116/124) | 39 (24/61) | 0.78 (0.71-0.85) | 76 | 75 | <0.0001 |
| HOXA9 | 92 (108/117) | 38 (18/48) | 0.70 (0.61-0.80) | 78 | 67 | <0.0001 |
| POU4F2 | 87 (104/120) | 47 (28/60) | 0.75 (0.68-0.83) | 76 | 64 | <0.0001 |
| TWIST1 | 89 (113/127) | 28 (17/60) | 0.71 (0.63-0.80) | 72 | 55 | <0.0001 |
| VIM | 90 (113/126) | 43 (24/56) | 0.72 (0.63-0.81) | 78 | 65 | <0.0001 |
| Cytology | 77 (88/115) | 60 (35/58) | 0.68 (0.61-0.76) | 79 | 56 | <0.0001 |

^{a}Some urine samples provided inconclusive results for some markers
^{b}Mann-Whitney U test

In comparison, the sensitivity of cytology was 77% and the specificity was 60%, the AUC (95% CI) was 0.68 (0.61-0.76), the PPV was 79%, and the NPV was 56%. Attempts to combine the markers resulted in lower specificity without much gain in sensitivity when combining two or more markers (results not shown). Of notice, urine samples from patients with recurrent tumors showed no significant associations between methylation and clinicopathologic variables as indicated in Table 12 below.

TABLE 12

| | | ZNF154 | EOMES | HOXA9 | POU4F2 | TWIST1 | VIM |
|---|---|---|---|---|---|---|---|
| Stage | pTa | 95% (74/78) | 91% (74/81) | 93% (74/80) | 85% (67/79) | 87% (74/85) | 89% (72/81) |
| | pT1 | 93% (26/28) | 96% (26/27) | 92% (22/24) | 93% (26/28) | 93% (26/28) | 93% (26/28) |
| | CIS | 100% (5/5) | 100% (5/5) | 100% (3/3) | 67% (2/3) | 75% (3/4) | 75% (3/4) |
| | T2-4 | 83% (10/12) | 100% (11/11) | 90% (9/10) | 90% (9/10) | 100% (10/10) | 92% (12/13) |
| | P-value* | 0.424 | 0.719 | 0.894 | 0.453 | 0.387 | 0.606 |
| Grade | I | 100% (9/9) | 88% (7/8) | 91% (10/11) | 81% (9/11) | 82% (9/11) | 89% (8/9) |
| | II | 94% (46/49) | 96% (48/50) | 93% (43/46) | 89% (42/47) | 88% (46/52) | 92% (46/50) |
| | III | 92% (60/65) | 92% (61/66) | 92% (55/60) | 85% (53/62) | 91% (58/64) | 88% (59/67) |
| | P-value | 1.000 | 0.398 | 1.000 | 0.725 | 0.642 | 0.657 |
| Age, years | ≦70 | 90% (43/48) | 96 (44/46) | 90% (38/42) | 86% (38/44) | 88% (44/50) | 93% (42/45) |
| | >70 | 96% (72/75) | 92% (72/78) | 93% (70/75) | 87% (66/76) | 90% (69/77) | 88% (71/81) |
| | P-value | 0.260 | 0.709 | 0.720 | 1.000 | 0.779 | 0.376 |
| Tumor size, cm | <3 cm | 93% (92/99) | 92% (94/102) | 91% (88/97) | 86% (85/99) | 89% (94/106) | 89% (92/103) |
| | >3 cm | 100% (12/12) | 100% (12/12) | 100% (11/11) | 100% (11/11) | 100% (12/12) | 100% (12/12) |
| | P-value | 1.000 | 0.597 | 0.594 | 0.353 | 0.609 | 0.602 |
| Cytology | Positive | 95% (74/78) | 94% (77/82) | 93% (68/73) | 89% (67/75) | 87% (70/79) | 90% (73/81) |
| | Negative | 85% (17/20) | 94% (17/18) | 95% (18/19) | 81% (17/21) | 90% (19/21) | 85% (17/20) |
| | P-value | 0.148 | 1.000 | 1.000 | 0.289 | 1.000 | 0.452 |
| Stix, nitrite | Positive | 83% (10/12) | 82% (9/11) | 100% (11/11) | 77% (10/13) | 85% (11/13) | 77% (10/13) |
| | Negative | 94% (101/107) | 95% (104/110) | 92 (94/102) | 87% (90/103) | 89% (97/109) | 92% (100/109) |
| | P-value | 0.185 | 0.156 | 1.000 | 0.386 | 0.645 | 0.118 |

*Fisher's exact test

The above data was obtained from cystoscopy results from the urine sampling visit. Using cystoscopy results from the following 12 months of follow-up, many of the samples formerly classified as false positives were determined to be true positives. The adjusted methylation marker values were: sensitivity 88% to 94%, specificity 37%-66%, AUC (95% CI) 0.78 (0.68-0.89) to 0.84 (0.77-0.91), PPV 81%-90%, and NPV 55%-78%. See Table 13 below.

TABLE 13

| | Sensitivity, % | Specificity, % | | | | |
|---|---|---|---|---|---|---|
| Gene | (pos./total^{a}) | (neg./total^{a}) | AUC (95% CI) | PPV, % | NPV, % | P value^{b} |
| ZNF154 | 94 (133/141) | 66 (29/44) | 0.83 (0.74-0.91) | 90 | 78 | <0.0001 |
| EOMES | 94 (131/139) | 52 (24/46) | 0.84 (0.77-0.91) | 86 | 75 | <0.0001 |
| HOXA9 | 93 (123/132) | 55 (18/33) | 0.78 (0.68-0.89) | 89 | 67 | <0.0001 |
| POU4F2 | 88 (120/136) | 64 (28/44) | 0.80 (0.72-0.89) | 88 | 64 | <0.0001 |
| TWIST1 | 90 (127/141) | 37 (17/46) | 0.79 (0.70-0.87) | 81 | 55 | <0.0001 |
| VIM | 91 (125/138) | 55 (24/44) | 0.80 (0.71-0.89) | 86 | 65 | <0.0001 |
| Cytology | 79 (99/126) | 74 (35/47) | 0.77 (0.69-0.84) | 89 | 56 | <0.0001 |

^{a}Some urine samples provided inconclusive results for some markers
^{b}Mann-Whitney U test

Adjusted cytology values were: sensitivity 79%, specificity 74%, AUC 0.77 (0.69-0.84), PPV 89% and NPV 56%.

Prognostic Value of Methylation Markers for Predicting Recurrences:

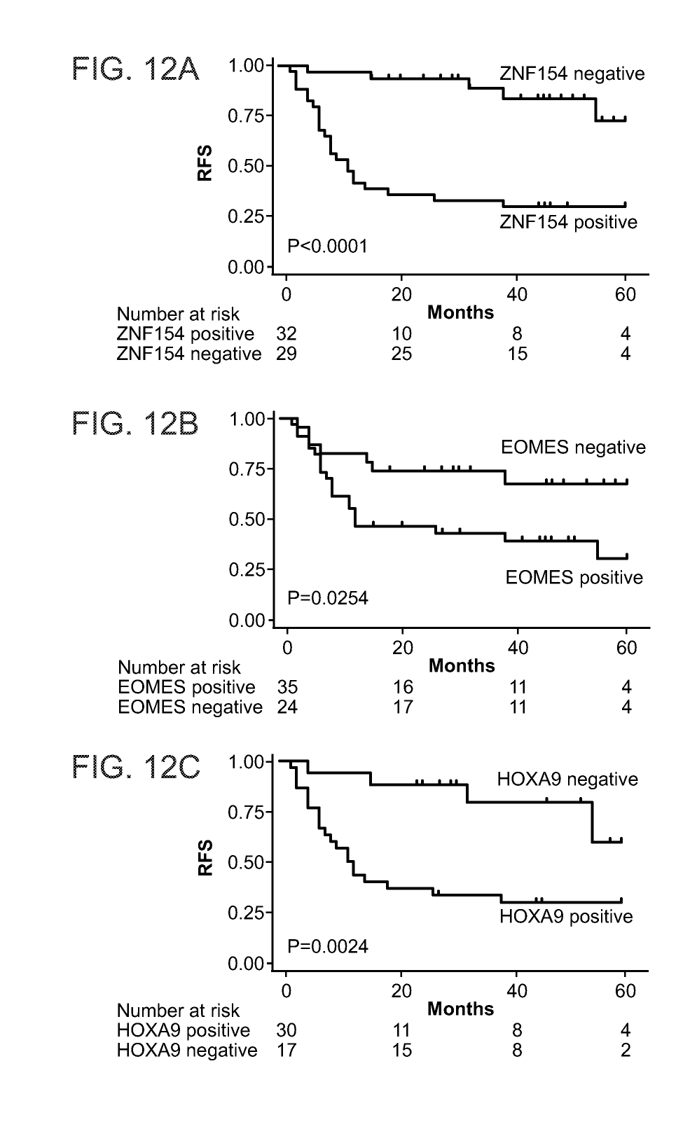

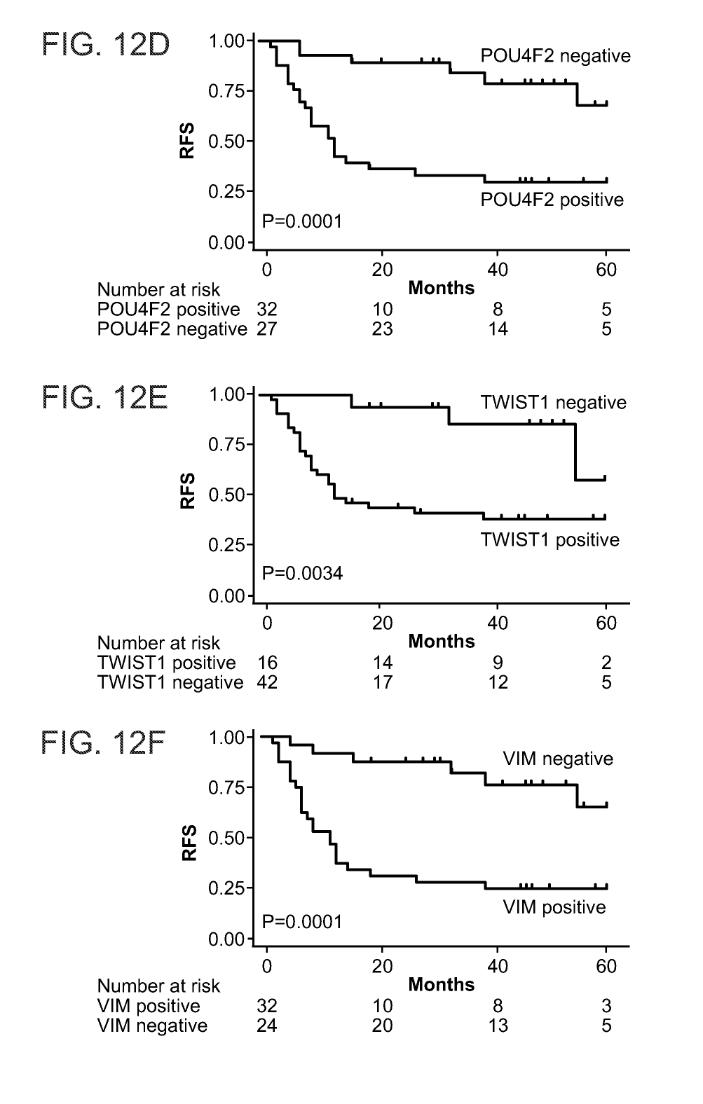

The prognostic value of the methylation markers at non-recurrent visits was assessed. For all markers, it was determined that a positive marker at a tumor negative visit was significantly associated with later tumor recurrence in a 24 or 60 month follow-up period (Log-Rank test, P≦0.0397). See FIG. 12. The most significant differences in a 24 month time-frame were observed for ZNF154 and POU4F2 (Log-Rank test, P<0.0001) where only 8% (2/26) and 12% (3/25) with no methylation experienced a recurrence within two years, respectively. For the methylation positive samples the percentage of patients with recurrence was 63% (20/32) for ZNF154 and 68% (21/31) for POU4F2. Univariate Cox regression analysis showed that methylation of ZNF154 (HR (95% CI)=13.9 (3.3-59.7), P<0.001), HOXA9 (HR (95% CI)=7.8 (1.8-33.7), P=0.006), POU4F2 (HR (95% CI)=8.5 (2.5-28.5), P=0.001), TWIST1 (HR (95% CI)=12.0 (1.6-88.6), P=0.015), and VIM (HR (95% CI)=8.0 (2.4-26.8), P=0.001) were significantly associated with poor recurrence free survival. See FIG. 12. Previous stage, grade, multiplicity, and CIS were not significantly associated with recurrence free survival (P>0.05). Thus, the presence of altered methylation of DNA in urine seemed to be strongly related to the prognosis.

Discovery of an Epigenetic Field Defect in Bladder Cancer Patients:

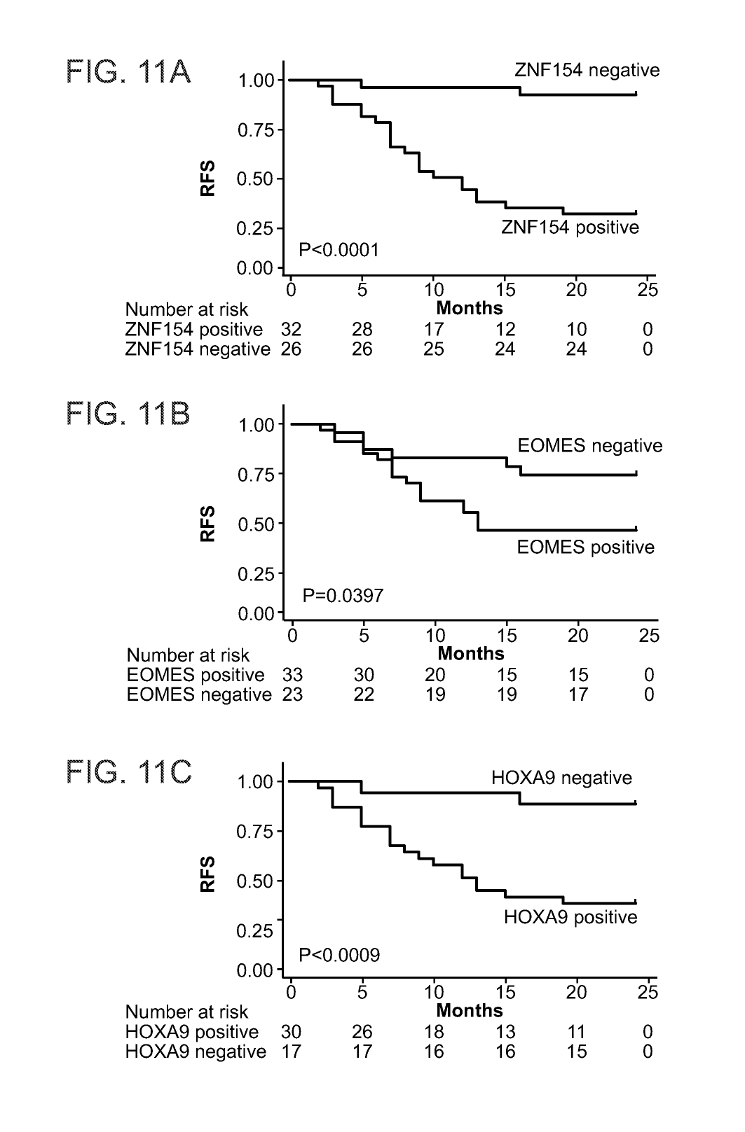

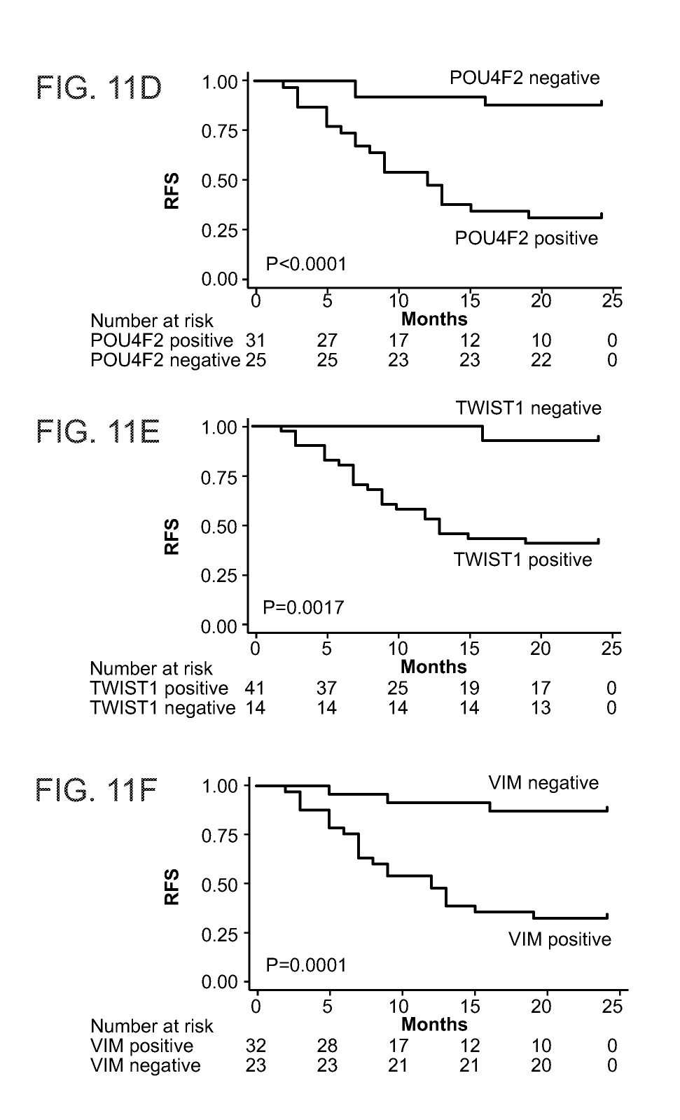

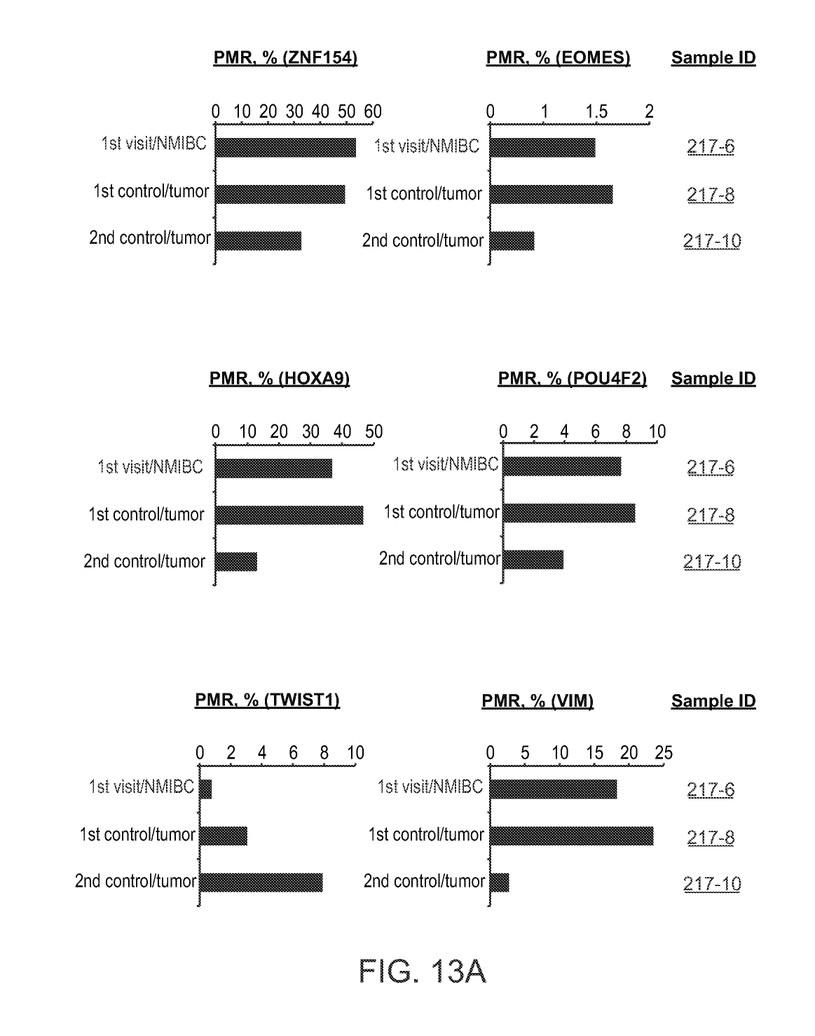

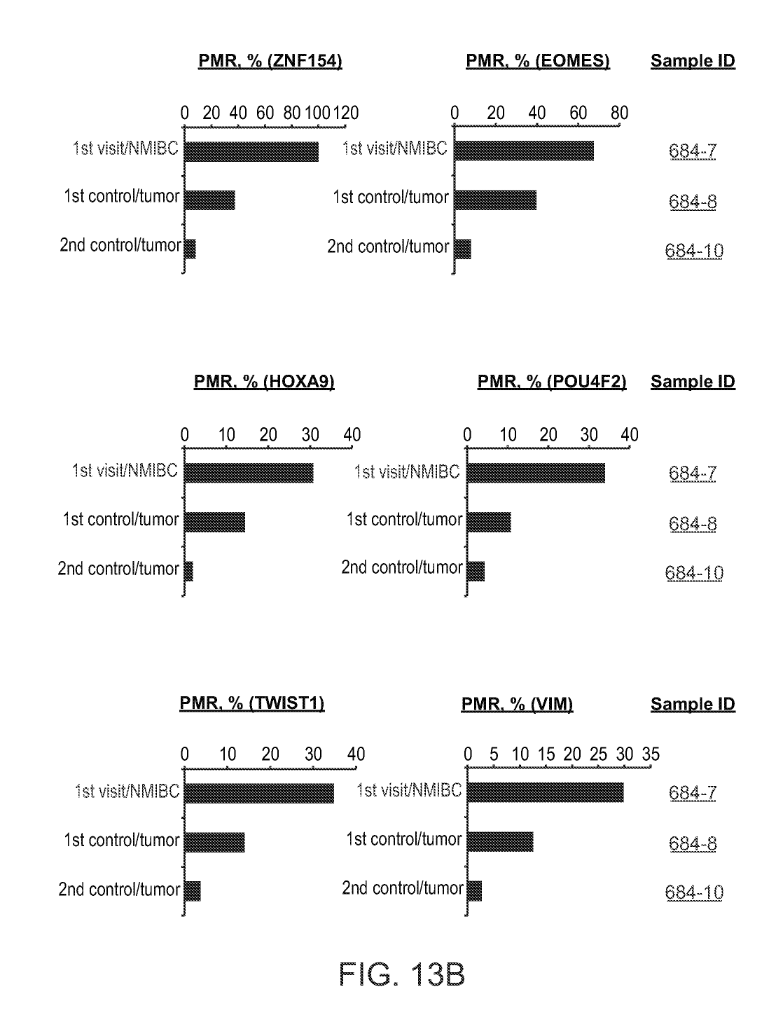

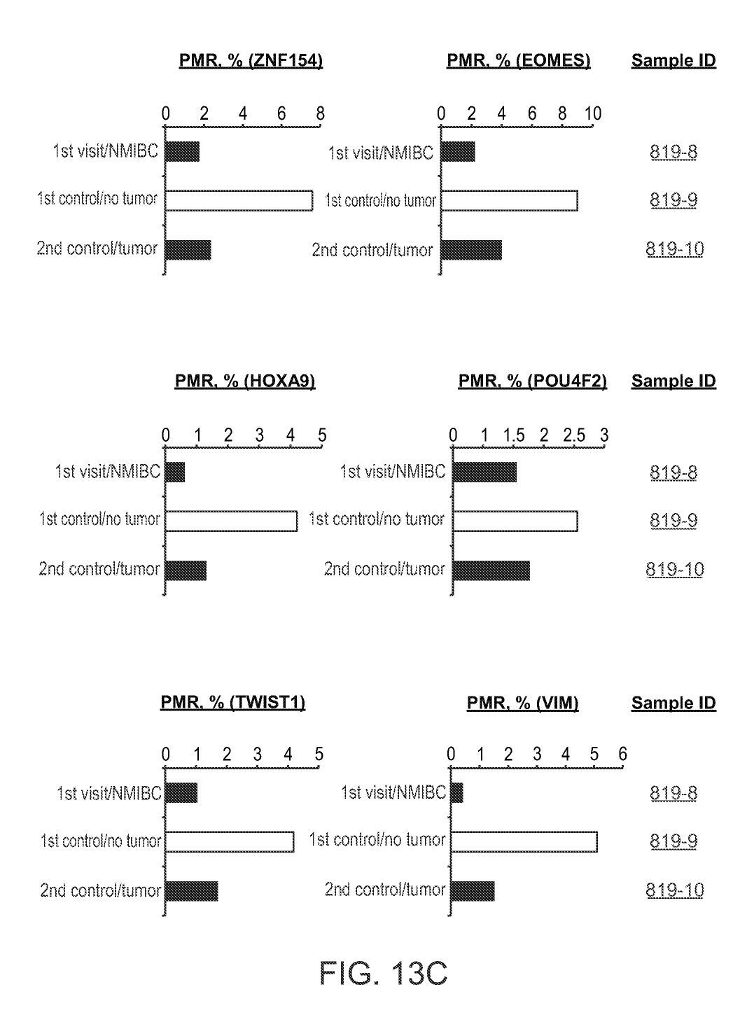

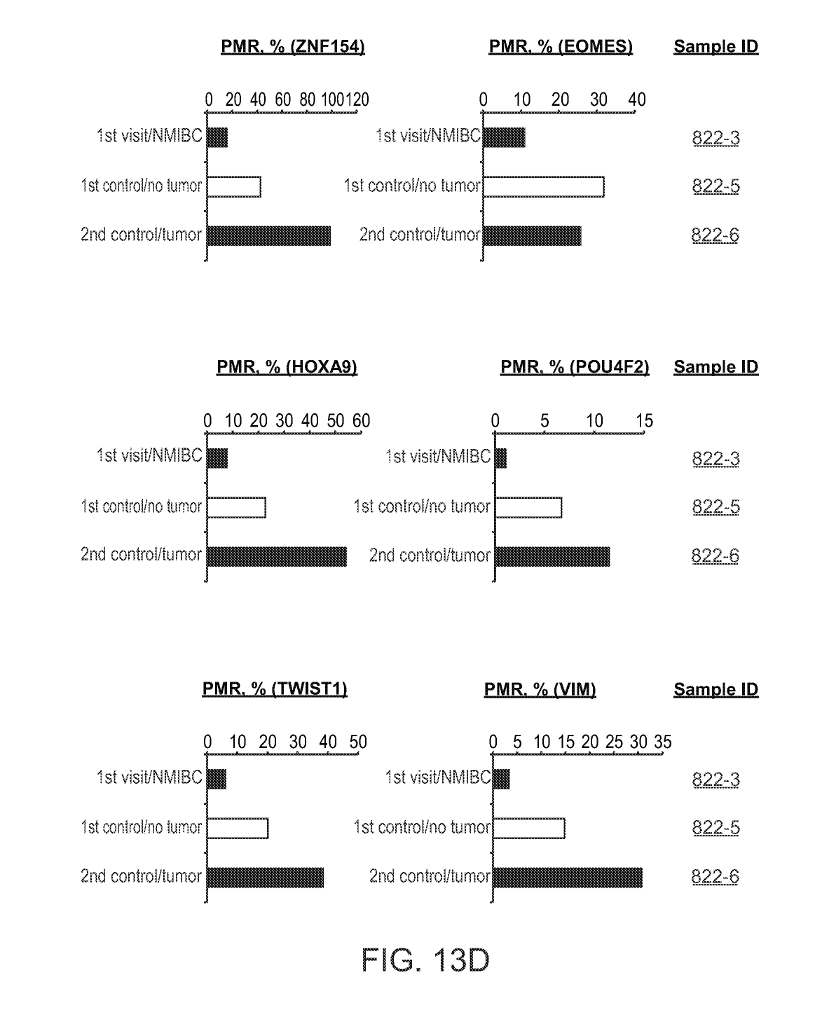

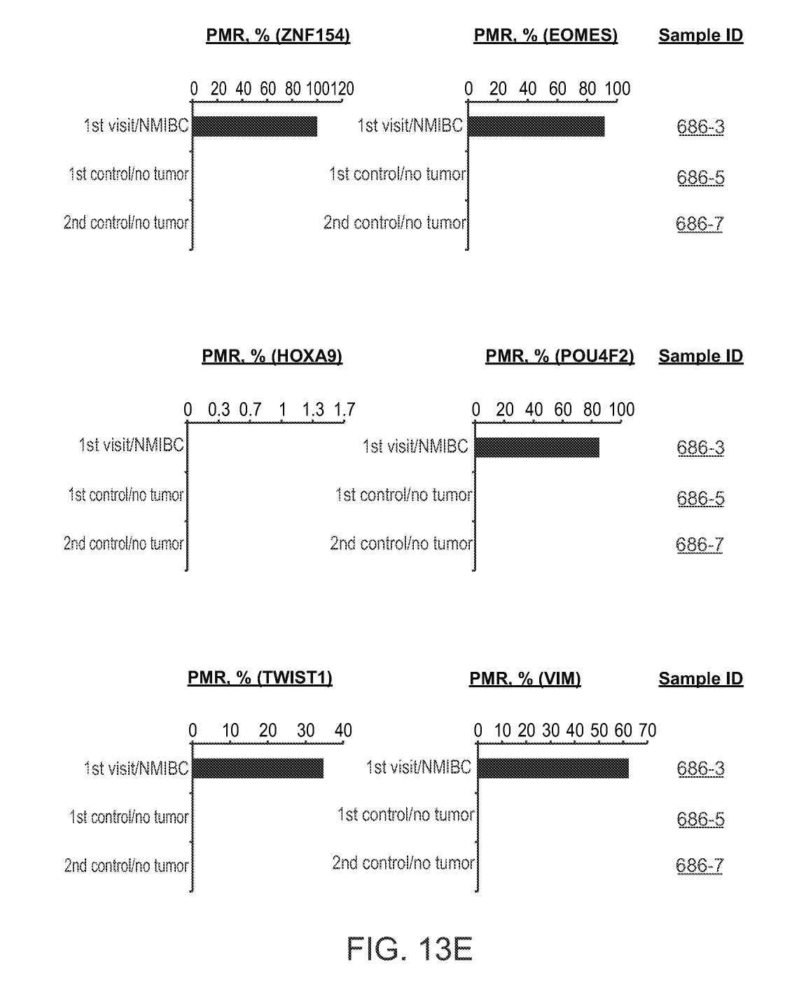

If the methylation of the biomarkers was confined to malignant cells forming tumors, the markers in urine should only be detected when a tumor was present, or occurring within a foreseeable future depending on the growth rate of the tumor. However, results indicate that even high urinary levels of methylation could be present at visits without recurrences. See FIG. 13, patients C and D. The patients were subdivided into; a group where methylation was present in the urine at the first visit and continued to be present in the follow-up period, although no tumor occurred (15 patients); a similar group where a tumor occurred later in the follow-up (20 patients); and a group in which the urine turned negative during the follow-up (18 patients) (see FIG. 13, patient E), and where no tumor occurred, or in whom this happened very rarely. According to one aspect described herein, the first two groups represent an epigenetic field defect, an urothelial methylator phenotype, and that these patients have a higher risk of recurrences and progression, compared to the methylation negative patients. The patients with the urothelial methylator phenotype have methylation levels in the urine during the disease course that are significantly higher (Mann-Whitney, P<0.05) than in non-cancer controls, although no tumor or CIS is detected. Although, some patients with methylation values close to 50% did not experience a recurrence within 5 years (as illustrated in FIG. 12). FIGS. 11 and 12 demonstrate that patients with hypermethylation of the marker genes have significantly increased risk of bladder cancer recurrence. As an example, one methylation positive patient with the most prolonged follow-up was diagnosed with CIS after 118 months, but no lesions developed in between.

SUPPLEMENTARY TABLE 1
Primer sequences and amplification protocols for sequencing
and methylation-sensitive high-resolution melting (MS-HRM).

| | Sense primer (5'-3') | |
|---|---|---|
| Gene | Antisense primer (5'-3') | Amplification protocols |
| ZNF154 | TAAGAGGTTGGTGTAAAGGGTTAT | 94° C. 4m, (94° C. 20s, 56° C. 30s, 72° C. 30s) × |
| (Sequencing) | CCCTATCCCAAACCTAAC | 35, 72° C. 10m |
| HOXA9 | GGAGGTTGGTTTAGGGTTTCTTAT | 94° C. 4m, (94° C. 20s, 56° C. 30s, 72° C. 30s) × |
| (Sequencing) | TAAATAACTATACTTCCCC | 40, 72° C. 3m |
| POU4F2 | GTTGGAGTTGGGAAGGGTACATCC | 94° C. 4m, (94° C. 20s, 56-64° C. 30s, 72° C. 30s) × |
| (Sequencing) | GTTCAAACTAACAACAAAA | 40, 72° C. 4m |
| ACOT11 | TAGGAGTTTTGTATAGAAAGTTTT | 94° C. 4m, (94° C. 20s, 56° C. 30s, 72° C. 30s) × |
| (Sequencing) | ACCAACCCCCTTCCCTAA | 40, 72° C. 3m |
| EOMES | GTTGGAAAATTGGGTTGGAAAGTA | 94° C. 4m, (94° C. 20s, 60° C. 30s, 72° C. 30s) × |
| (Sequencing) | AATTAAACTCCAACTACTTATTTC | 40, 72° C. 3m |
| | TTC | |
| PCDHGA12 | GATTGTGTAGTAATTGGTTAGGAT | 94° C. 4m, (94° C. 20s, 63° C. 30s, 72° C. 30s) × |
| (Sequencing) | TTATTCTACCCTTAACCACTAAAA | 40, 72° C. 3m |
| | TCAA | |
| CA3 | TTATATGTTGTTTGTAAAGGGAGT | 94° C. 4m, (94° C. 20s, 63° C. 30s, 72° C. 30s) × |
| (Sequencing) | TCTCCTTCCTCCATACATTCTTA | 40, 72° C. 3m |
| PTGDR | GAGTTTAGATAGGAGGTTTTTGTA | 94° C. 4m, (94° C. 20s, 62° C. 30s, 72° C. 30s) × |
| (Sequencing) | CCAACACTCCAATACCATAAC | 40, 72° C. 3m |
| HIST1H4F | GTGAGGTTTAGTTATTAAAGTTAA | 94° C. 4m, (94° C. 20s, 63° C. 30s, 72° C. 30s) × |
| (Sequencing) | CAACATACAAAACATCAC | 40, 72° C. 3m |
| SLC22A12 | GAGGTGGGTATATAGGGGTAATAT | 94° C. 4m, (94° C. 20s, 56° C. 30s, 72° C. 30s) × |
| (Sequencing) | CACTTAAAATAACTCCAACTAA | 35, 72° C. 3m |
| GRM4 | AGATGGGGATATTATATTGGGAGC | 94° C. 4m, (94° C. 20s, 63° C. 30s, 72° C. 30s) × |
| (Sequencing) | TCAAAAAACCAACCAAAACACTA | 40, 72° C. 3m |
| CHRNB1 | GAATAAGTGTAGTTTTGGTGTTTG | 94° C. 4m, (94° C. 20s, 59° C. 30s, 72° C. 30s) × |
| (Sequencing) | GTACTATCCTCCAACAACAAATAC | 35, 72° C. 10m |
| | ACA | |
| ZNF154 | TGTGTTTATCGGATTAGAGATAGT | 94° C. 4m, (94° C. 5s, 58° C. 5s, 72° C. s) × |
| (MS-HRM) | AGAGCCAAACCTAACGTAAATCCC | 40, 72° C. 30s |
| | CCAAAA | |
| POU4F2 | GGGTTGTGCGAAGTTGAGTTTATA | 94° C. 4m, (94° C. 5s, 55° C. 5s, 72° C. s) × |
| (MS-HRM) | CATCCGTTCAAACTAACAACAAAA | 40, 72° C. 30m |
| HOXA9 | GAGTTTACGTAGTAGTTGTTTAGG | 94° C. 4m, (94° C. 5s, 58° C. 5s, 72° C. s) × |
| (MS-HRM) | GTTTCCCCCGATACCACCAAATTA | 40, 72° C. 30s |
| | TTACATA | |
| ACOT11 | GTTTTGTATAGAAAGTTTTAGTGT | 94° C. 4m, (94° C. 5s, 58° C. 5s, 72° C. s) × |
| (MS-HRM) | CCCAATAAACGAACACAAACC | 40, 72° C. 30s |
| EOMES | GGAAGTAGAGTTTCGATATTTTAG | 94° C. 4m, (94° C. 5s, 59° C. 5s, 72° C. s) × |
| (MS-HRM) | TAATATTCGCTATTAATAAACAAT | 40, 72° C. 30s |
| | TACAA | |
| PCDHGA12 | TGTTTATTAATCGGGGAGAGAAAA | 94° C. 4m, (94° C. 5s, 55° C. 5s, 72° C. s) × |
| (MS-HRM) | CCGTTATTTCCACATACTCCCAA | 40, 72° C. 30s |
| CA3 | TAAATAAACGAGTTTTTTTTAGTT | 94° C. 4m, (94° C. 5s, 58° C. 5s, 72° C. s) × |
| (MS-HRM) | TTTGCTAACTACGATCTCTAAACA | 40, 72° C. 30s |
| | CTTA | |
| TBX4 | GAGTGATTCGTTGTGTGTTGTGTT | 94° C. 4m, (94° C. 5s, 62° C. 5s, 72° C. s) × |
| (MS-HRM) | ACGTTAACTACGCTAACCTCTCC | 40, 72° C. 30s |
| BRF1 | AGTTATTCGTGGTTATTTGTGGTT | 94° C. 4m, (94° C. 5s, 55° C. 5s, 72° C. s) × |
| (MS-HRM) | ATTCGCATTTCTTTTTAAACTCAT | 40, 72° C. 30s |
| | TCCTAA | |
| CHRNB1 | CGTTATTGTTTTTTCGGGGTTTAT | 94° C. 4m, (94° C. 5s, 58° C. 5s, 72° C. s) × |
| (MS-HRM) | ATTTATTCGAACTCCTCGTCACTT | 40, 72° C. 30s |
| | CCCCTATACTA | |
| PTGDR | CGTTTTTTCGTAGTTTTTATTTTA | 94° C. 4m, (94° C. 5s, 56° C. 5s, 72° C. s) × |
| (MS-HRM) | GTTTTTCGCCGAATTACCTTTTTC | 40, 72° C. 30s |
| | CACAA | |
| SCARF2 | GGAATCGGTTAAGGGAGTGGCCGC | 94° C. 4m, (94° C. 5s, 61° C. 5s, 72° C. s) × |
| (MS-HRM) | ACACCCCAACACTACAAA | 40, 72° C. 30s |
| SOCS3 | GAGGTCGCGAAGTAGTTGTAGTAA | 94° C. 4m, (94° C. 5s, 58° C. 5s, 72° C. s) × |
| (MS-HRM) | AATCTTAAAACGCAAACTAATATC | 40, 72° C. 30s |
| | CAAA | |
| ZNF135 | GCGTTTTAAGCGGTATTTATTTCG | 94° C. 4m, (94° C. 5s, 50° C. 5s, 72° C. s) × |
| (MS-HRM) | TTCGTAAACTCTCAATACTACTAA | 40, 72° C. 30s |
| | A | |
| LHX2 | TTTTTGGGGTGCGTTTTTAGTTTA | 94° C. 4m, (94° C. 5s, 62° C. 5s, 72° C. s) × |
| (MS-HRM) | AGGCAAACTTCGAAAAAACCCCTA | 40, 72° C. 30s |
| | CTCCAA | |

SUPPLEMENTARY TABLE 2
Performance of tumor marker candidates identified by Infinium array and
validated by MS-HRM.

| | Discovery (Infinium) | | | | Validation (MS-HRM) | | | | |
|---|---|---|---|---|---|---|---|---|---|
| | | | Sensitivity, | | | | Sensitivity, % | Specificity, % | |
| | Δβ- | P- | % (positive/ | Specificity, % | | | (positive/ | (negative/ | Kappa- |
| Gene | value | value | total) | (negative/total) | ΔMethylation, % | P-value | total) | total) | value |
| ZNF154 | 0.52 | 0.0024 | 85 (22/26) | 100 (6/6) | 62 | <0.0001 | 91 (50/55) | 100 (8/8) | 0.95 |
| HOXA9 | 0.50 | 0.0003 | 92 (24/26) | 100 (6/6) | 56 | 0.0001 | 89 (47/53) | 100 (8/8) | 0.91 |
| POU4F2 | 0.47 | 0.0004 | 92 (24/26) | 100 (6/6) | 47 | <0.0001 | 96 (53/55) | 100 (8/8) | 1.00 |
| EOMES | 0.44 | 0.0004 | 88 (23/26) | 100 (6/6) | 30 | 0.0005 | 80 (44/55) | 100 (8/8) | 0.84 |
| ACOT11 | 0.44 | 0.0004 | 92 (24/26) | 100 (6/6) | 41 | 0.0001 | 91 (49/54) | 100 (7/7) | 0.71 |
| PCDHGA | 0.43 | 0.0001 | 96 (25/26) | 100 (6/6) | 43 | <0.0001 | 95 (52/55) | 100 (8/8) | 0.94 |
| 12 | | | | | | | | | |
| CA3 | 0.42 | 0.0001 | 88 (23/26) | 100 (6/6) | 42 | <0.0001 | 96 (52/54) | 100 (8/8) | 0.94 |
| PTGDR | 0.39 | 0.0218 | 58 (15/26) | 100 (6/6) | 24 | 0.0039 | 58 (32/55) | 100 (8/8) | 0.90 |

The delta methylation percentage values are the MS-HRM methylation values of the tumors minus the normals.
P-values were calculated using a two-sample Wilcoxon rank-sum (Mann-Whitney) test.

SUPPLEMENTARY TABLE 3
Performance of stage and invasive markers as well as the candidate
markers of progression identified by Infinium arrays and validated by MS-HRM.

| | Discovery (Infinium) | | | | | Validation (MS-HRM) | | | | |
|---|---|---|---|---|---|---|---|---|---|---|
| | | | | Specificity, % | | | | Sensitivity, % | Specificity, % | |
| | Δβ- | P- | Sensitivity, % | (negative/ | Infinium | | P- | positive/ | (negative/ | Kappa- |
| | value | value | (positive/total) | total) | targetID | ΔMethlylation, % | value | total) | total) | value |
| T2-4- | | | | | | | | | | |
| Ta(stable) | | | | | | | | | | |
| CHRNB1 | −0.38 | 0.0030 | 100 (8/8) | 75 (6/8) | cg18884137 | −27 | 0.0022 | 67 (10/15) | 80 (12/15) | 0.76 |
| PTGDR | 0.59 | 0.0019 | 89 (8/9) | 100 (8/8) | cg09516965 | 13 | 0.10 | 47 (7/15) | 80 (12/15) | 0.86 |
| ZNF135 | 0.40 | 0.0011 | 89 (8/9) | 88 (7/8) | cg1663854 | 11 | 0.33 | 67 (10/15) | 60 (9/15) | 0.86 |
| BRF1 | −0.40 | 0.0047 | 88 (6/8) | 100 (8/8) | cg16313343 | −25 | 0.016 | 33 (5/15) | 80 (12/15) | 0.86 |
| TBX4 | 0.46 | 0.0003 | 100 (8/8) | 88 (7/8) | cg18536148 | 35 | 0.0006 | 87 (13/15) | 73 (11/15) | 0.58 |
| SOCS3 | −0.34 | 0.0281 | 75 (6/8) | 88 (7/8) | cg27637521 | −37 | 0.0157 | 67 (10/15) | 80 (12/15) | 0.77 |
| T1- | | | | | | | | | | |
| Ta(stable) | | | | | | | | | | |
| PTGDR | 0.51 | 0.0055 | 78 (7/9) | 100 (8/8) | cg09516965 | −16 | 0.023* | 47 (7/15) | 20 (3/15) | 0.86 |
| ZNF135 | 0.37 | 0.0025 | 89 (8/9) | 88 (7/8) | cg1663854 | 15 | 0.27 | 73 (11/15) | 60 (9/15) | 0.86 |
| TBX4 | 0.38 | 0.003 | 100 (9/9) | 88 (7/8) | cg18536148 | 24 | 0.005 | 73 (11/15) | 87 (13/15) | 0.58 |
| Ta(prog)- | | | | | | | | | | |
| Ta(stable) | | | | | | | | | | |
| ZNF135 | 0.34 | 0.0206 | 78 (7/9) | 88 (7/8) | cg1663854 | 12 | 0.19 | 80 (8/10) | 60 (9/15) | 0.86 |
| BRF1 | −0.34 | 0.0206 | 78 (7/9) | 100 (8/8) | cg16313343 | −1 | 1.0 | 50 (4/8) | 100 (15/15) | 0.86 |
| TBX4 | 0.39 | 0.002 | 100 (9/9) | 88 (7/8) | cg18536148 | 24 | 0.037 | 50 (5/10) | 93 (14/15) | 0.58 |
| T2-4-T1 | | | | | | | | | | |
| SCARF2 | −0.34 | 0.0262 | 86 (6/7) | 86 (6/7) | cg14785479 | −22 | 0.004 | 67 (10/15) | 60 (9/15) | 0.68 |
| SOCS3 | −0.39 | 0.0111 | 88 (7/8) | 67 (6/9) | cg27637521 | −26 | 0.070 | 67 (10/15) | 67 (10/15) | 0.77 |
| Invasive- | | | | | | | | | | |
| Non- | | | | | | | | | | |
| invasive | | | | | | | | | | |
| SOCS3 | −0.31 | 0.0148 | 88 (7/8) | 72 (13/18) | cg27637521 | −29 | 0.0187 | 67 (10/15) | 73 (29/40) | 0.77 |

The delta methylation percentage values are the MS-HRM methylation values of the more advanced tumors minus the lesser advances tumors.
P-values were calculated using a two-sample Wilcoxon rank-sum (Mann-Whitney) test.
*Statistically significant difference between groups, but hypomethylated instead of hypermethylated.

SUPPLEMENTARY TABLE 4
Associations between methylation markers in tumor samples.
The methylation values were included in five percent intervals. The data
were obtained from the independent validation set (N = 55).
The Spearman rho coefficients are shown.

| Gene | ZNF154 | HOXA9 | POU4F2 | EOMES | CA3 | PCDHGA12 | ACOT11 | PTGDR |
|---|---|---|---|---|---|---|---|---|
| ZNF154 | 1 | 0.42* | 0.48* | 0.52* | 0.34* | 0.49* | 0.35* | 0.42* |
| HOXA9 | | 1 | 0.60* | 0.37* | 0.31* | 0.32* | 0.38* | 0.44* |
| POU4F2 | | | 1 | 0.61* | 0.39* | 0.50* | 0.29* | 0.45* |
| EOMES | | | | 1 | 0.52* | 0.59* | 0.27 | 0.63* |
| CA3 | | | | | 1 | 0.45* | 0.33* | 0.49* |
| PCDHGA12 | | | | | | 1 | 0.30* | 0.46* |
| ACOT11 | | | | | | | 1 | 0.38* |
| PTGDR | | | | | | | | 1 |

*Rho coefficients from Spearman correlation coefficient with a p-value < 0.05.

SUPPLEMENTARY TABLE 5
Performance of the methylation assays for ZNF154 + HOXA9 +
POU4F2 + EOMES in DNA from urine specimens using cut-off
point obtained by ROC analysis.

| | | Sensitivity, % | Specificity, % | | | | |
|---|---|---|---|---|---|---|---|
| | | (pos./total) | (neg./total) | AUC (95 CI) | PPV % | NPV % | P-value** |
| Stage | pTa | 80 (47/59) | 97 (57/59) | 0.88 (0.82-0.94) | 96 | 83 | <0.0001 |
| | pT1 | 85 (23/27) | 97 (57/59) | 0.91 (0.84-0.98) | 92 | 93 | <0.0001 |
| | pT2-4 | 83 (24/29) | 97 (57/59) | 0.90 (0.82-0.97) | 92 | 92 | <0.0001 |
| Grade | I | 71 (12/17) | 97 (57/59) | 0.84 (0.72-0.95) | 86 | 92 | <0.0001 |
| | II | 78 (29/37) | 97 (57/59) | 0.88 (0.80-0.95) | 94 | 88 | <0.0001 |
| | III + IV | 87 (53/61) | 97 (57/59) | 0.92 (0.87-0.97) | 96 | 88 | <0.0001 |
| Cytology | Positive | 95 (37/39) | N/A* | 0.96 (0.92-1.00) | 100 | N/A | <0.0001 |
| | Negative | 87 (13/15) | N/A* | 0.92 (0.82-1.00) | 100 | N/A | <0.0001 |

*Not applicable.
**Mann-Whitney U test

SUPPLEMENTARY TABLE 6
Analytical sensitivity of methylation markers by MS-HRM in
DNA from urine specimens.

| | Methylation events in urine/ | | | |
|---|---|---|---|---|
| | Methylation events detected in tumors (%) | | | |
| Gene | Total | Ta | T1 | T2-4 |
| ZNF154 | 25/30 (83) | 4/7 (57) | 9/10 (90) | 12/13 (92) |
| HOXA9 | 28/29 (97) | 6/7 (86) | 10/10 (100) | 13/13 (100) |
| POU4F2 | 26/32 (81) | 6/8 (75) | 8/10 (80) | 12/13 (92) |
| EOMES | 26/27 (96) | 5/6 (83) | 9/9 (100) | 12/12 (100) |
| ZNF154 + | 31/33 (94) | 8/10 (80) | 10/10 (100) | 13/13 (100) |
| HOXA9 + | | | | |
| POU4F2 + EOMES | | | | |

SUPPLEMENTARY TABLE 7
Associations between methylation markers and stage, grade,
and age in urine specimens. Methylation values were
dichotomized as positive or negative.

| | | ZNF154 | HOXA9 | POU4F2 | EOMES | Combined |
|---|---|---|---|---|---|---|
| Stage | pTa | 50% (27/54) | 75% (38/51) | 61% (35/57) | 57% (26/46) | 80% (47/59) |
| | pT1 | 81% (22/27) | 78% (21/27) | 74% (20/27) | 77% (20/26) | 85% (23/27) |
| | pT2-4 | 66% (19/29) | 69% (20/29) | 69% (20/29) | 79% (23/29) | 83% (24/29) |
| | P-value | 0.019 | 0.780 | 0.511 | 0.074 | 0.903 |
| Grade | I | 29% (4/14) | 64% (9/14) | 53% (9/17) | 50% (6/12) | 71% (12/17) |
| | II | 51% (18/35) | 78% (25/32) | 60% (21/35) | 55% (16/29) | 78% (29/37) |
| | III-IV | 75% (46/61) | 74% (45/61) | 74% (45/61) | 78% (47/60) | 87% (53/61) |
| | P-value | 0.002 | 0.608 | 0.163 | 0.036 | 0.221 |
| Age, years | 39-65 | 53% (21/40) | 74% (28/38) | 59% (24/41) | 59% (20/34) | 81% (35/43) |
| | 65+ | 67% (47/70) | 74% (51/69) | 71% (51/72) | 73% (49/67) | 82% (59/72) |
| | P-value | 0.155 | 1.000 | 0.216 | 0.177 | 1.000 |
| Cytology | Positive | 77% (30/39) | 82% (32/39) | 82% (32/39) | 85% (33/39) | 95% (37/39) |
| | Negative | 36% (5/14) | 93% (13/14) | 50% (7/14) | 54% (7/13) | 87% (13/15) |
| | P-value | 0.009 | 0.665 | 0.033 | 0.051 | 0.306 |
| Stix, nitrite | Positive | 60% (3/5) | 60% (3/5) | 60% (3/5) | 40% (2/5) | 60% (3/5) |
| | Negative | 62% (64/104) | 74% (75/102) | 67% (71/106) | 69% (67/97) | 83% (90/108) |
| | P-value* | 1.000 | 0.611 | 1.000 | 0.325 | 0.214 |

*Fisher's exact test.

SUPPLEMENTARY TABLE 8
Pathway analysis of genes with differential methylation between patients
with low grade and high grade tumors, and between stable Ta disease and
Ta tumors that progress based on analysis of metachronous tumors. The
p-values are adjusted for multiple testing by Benjamini-Hochberg (B-H).

| | | |
|---|---|---|
| P-value | | Patient |
| (Adj B-H) | Epigenetic regulated genes | ID |

Stable Ta group versus progressing disease group
Top network associated functions

| | | | |
|---|---|---|---|
| Cell movement of | 1.65E−10 | CCL27, CD9, IL1RN, | |
| eukaryotic cells | | STAT5A | |
| Tumorigenesis | 3.37E−08 | ALDH1A2, CCL27, CD9, | |
| | | GSTM2, H19, IL1RN, | |
| | | KRT13, STAT5A, | |
| | | TM4SF1 | |
| Growth of cancer | 4.46E−07 | CCL27, IL1RN | |
| cells | | | |
| Apoptosis | 1.48E−06 | ALDH1A2, BRF1, CCL27, | |
| | | CD9, IL1RN, STAT5A | |
| Proliferation of cells | 3.91E−06 | ALDH1A2, CD9, H19, | |
| | | IL1RN, LEFTY1, STAT5A | |
| Development of cells | 1.20E−05 | ALDH1A2, CD9, IL1RN, | |
| Canonical pathways | | STAT5A | |
| | 9.96E−06 | DRD5, EDNRA, GNAO1, | ID 1 |
| | | GRM8, MAPK1, PDE3B, | |
| | | PIK3R1, EDNRB, | |
| | | NFKBIE, PDE8B, | |
| | | ADORA2B, AGTR1, | |
| | | ADRA1A | |
| G-protein coupled | 3.14E−03 | ADRA1A, ADRA1B, | ID 2 |
| receptor signaling | | ADRA1D, AGTR2, DRD2, | |
| | | EDNRB, HTR1B, | |
| | | NFKBIE, PDE3A, | |
| | | PDE8B | |
| | 1.56E−02 | ADORA2B, ADRA1D, | ID 3 |
| | | ADRB3, AGTR1, OPRM1, | |
| | | PI3KCG, RELA, DRD2 | |

High grade versus low grade
Top network associated functions

| | | |
|---|---|---|
| Apoptosis of | 7.20E−41 | 47 genes |
| eukaryotic cells | | |
| Cell death of | 1.42E−40 | 49 genes |
| eukaryotic cells | | |
| Tumorigenesis | 1.84E−39 | 61 genes |
| Apoptosis | 2.01E−39 | 40 genes |
| Proliferation of cells | 1.81E−30 | 32 genes |
| Development of cells | 1.45E−28 | 10 genes |

Primer and DNA Sequences for Methylation Markers:

| | PTGDR | ZNF135 | TBX4 | BRF1 |
|---|---|---|---|---|
| Probe | CGCCATGAAGTCGCCGTTC | AGTGACTGGGCCTGGT | CGGGCACAGCACACAA | CGCCCTCAAATATCTG |
| source | TACCGCTGCCAGAACACCA | GAAACAGGGCGCGCGG | CGAGTCACTCGCCCAG | CAGGTGCTGTTCACAA |
| sequence | CCTCTGTGGAAA | GGTGCCTGGGCATCAA | GTCCACACTCGCCAGA | TCGCCATAGGGCCGGT |
| | | CG | GG | GA |

Bisulfite sequencing primers

| | | | | |
|---|---|---|---|---|
| Forward | GAGTTTAGATAGGAGGTTT | GAGTGATTGGGTTTGG | Not Done | Not Done |
| (5'-3') | TTGT | TGAAAT | | |
| Reverse | ACCAACACTCCAATACCAT | CAATATAAAATCTCCA | Not Done | Not Done |
| (5'-3') | AAC | TAACTACCTCAA | | |

MS-HRM primers

| | | | | |
|---|---|---|---|---|
| Forward | CGTTTTTTCGTAGTTTTTA | GCGTTTTAAGCGGTAT | GAGTGATTCGTTGTGT | AGTTATTCGTGGTTAT |
| (5'-3') | TTTTAGTTTTT | TTATTT | GTTGTGTT | TTGTGGTTATT |
| Reverse | CGCCGAATTACCTTTTTCC | CGTTCGTAAACTCTCA | ACGTTAACTACGCTAA | CGCATTTCTTTTTAAA |
| (5'-3') | ACAA | ATACTACTAAA | CCTCTCC | CTCATTCCTAA |

MS-HRM sequence

| | | | | |
|---|---|---|---|---|
| Untreated | CGCCCTTCCGCAGCCTTCA | GCGTTCCAAGCGGCAC | GAGTGACTCGTTGTGT | AGTCATTCGTGGCCAC |
| | CTCCAGCCCTCTGCTCCCG | TTATCCCGCGTTGATG | GCTGTGCCCGCAGGAG | CTGTGGTTACCCGTGA |
| | CACGCCATGAAGTCGCCGT | CCCAGGCACCCCGCGC | ATGCTGCAGGATAAGG | GTCACCTCGCTGTGCC |
| | TCTACCGCTGCCAGAACAC | GCCCTGTTCACCAGGC | GCCTCTCCGAGAGCGA | CCCTGCCCAGAGCGGG |
| | CACCTCTGTGGAAAAAGGC | CCAGTCACTCCAGCTC | GGAGGCCTTCCGGGCC | AACCCTGGCTGCGCAC |
| | AACTCGGCG | CAGCAGCACTGAGAGC | CCGGGCCCAGCGCTCG | GCCCTCAAATATCTGC |
| | | TCACGAACG | GAGAGGCCAGCGCAGC | AGGTGCTGTTCACAAT |
| | | | CAACGC | CGCCATAGGGCCGGTG |
| | | | | ACATACCCAGGAATGA |
| | | | | GCCTAAAAAGAAATGC |
| | | | | G |
| Bisulfite | CGTTTTTTCGTAGTTTTTA | GCGTTTTAAGCGGTAT | GAGTGATTCGTTGTGT | AGTTATTCGTGGTTAT |
| converted | TTTTAGTTTTTTGTTTTCG | TTATTTCGCGTTGATG | GTTGTGTTCGTAGGAG | TTGTGGTTATTCGTGA |
| | TACGTTATGAAGTCGTCGT | TTTAGGTATTTCGCGC | ATGTTGTAGGATAAGG | GTTATTTCGTTGTGTT |
| | TTTATCGTTGTTAGAATAT | GTTTTGTTTTATTAGG | GTTTGTTCGAGAGCGA | TTTTGTTTAGAGCGGG |
| | TATTTTTGTGGAAAAAGGT | TTTAGTTATTTTAGTT | GGAGGTTTTTCGGGTT | AATTTTGGTTGCGTAC |
| | | TTAGTAGTATTGAGAG | TCGGGTTTAGCGTTCG | GTTTTTAAATATTTGT |
| | | T | GAGAGGT | A |

Sequenced sequence

| Untreated | GAGTTTAGATAGGAGGTTC | GAGTGACTGGGCCTGG | | |
|---|---|---|---|---|
| | CTGCCGTGGGGAACACCCC | TGAAACAGGGCGCGCG | | |
| | GCCGCCCTCGGAGCTTTTT | GGGTGCCTGGGCATCA | | |
| | CTGTGGCGCAGCTTCTCCG | ACGCGGGATAAGTGCC | | |
| | CCCGAGCCGCGCGCGGAGC | GCTTGGAACGCCGTGA | | |
| | TGCCGGGGGCTCCTTAGCA | GCTCCCGGCGCGACCA | | |
| | CCCGGGCGCCGGGGAGCAC | CACGGGCTTTCGTGTT | | |
| | CCGGGGCGCCGGGGCCCTC | GGAAACTCTACCGTCA | | |
| | GCCCTTCCGCAGCCTTCAC | GTTTTACTGCTGCAAA | | |
| | TCCAGCCCTCTGCTCCCGC | CAAAGCAATGATTTTT | | |
| | ACGCCATGAAGTCGCCGTT | TCAAAACACATACTTT | | |
| | CTACCGCTGCCAGAACACC | CAACCAAAACATTCAC | | |
| | ACCTCTGTGGAAAAAGGCA | CAAGATCGCACACACG | | |
| | ACTCGGCGGTGATGGGCGG | GAGTCTTTGCATTTTC | | |
| | GGTGCTCTTCAGCACCGGC | CACACTGAGGCAGCCA | | |
| | CTCCTGGGCAACCTGCTGG | TGGAGACTTCACACTG | | |
| | CCCTGGGGCTGCTGGCGCG | | | |
| | CTCGGGGCTGGGGTGGTGC | | | |
| | TCGCGGCGTCCACTGCGCC | | | |
| | CGCTGCCCTCGGTCTTCTA | | | |
| | CATGCTGGTGTGTGGCCTG | | | |
| | ACGGTCACCGACTTGCTGG | | | |
| | GCAAGCGCCTCCTAAGCCC | | | |
| | GGTGGTGCTGGCTGCCTAC | | | |
| | GCTCAGAACCGGAGTCTGC | | | |
| | GGGTGCTTGCGCCCGCATT | | | |
| | GGACAACTCGTTGTGCCAA | | | |
| | GCCTTCGCCTTCTTCATGT | | | |
| | CCTTCTTTGGGCTCTCCTC | | | |
| | GACACTGCAACTCCTGGCC | | | |
| | ATGGCACTGGAGTGCTGGC | | | |
| Bisulfite | GAGTTTAGATAGGAGGTTT | GAGTGATTGGGTTTGG | | |
| converted | TTGTCGTGGGGAATATTTC | TGAAATAGGGCGCGCG | | |
| | GTCGTTTTCGGAGTTTTTT | GGGTGTTTGGGTATTA | | |
| | TTGTGGCGTAGTTTTTTCG | ACGCGGGATAAGTGTC | | |
| | TTCGAGTCGCGCGCGGAGT | GTTTGGAACGTCGTGA | | |
| | TGTCGGGGGTTTTTTAGTA | GTTTTCGGCGCGATTA | | |
| | TTCGGGCGTCGGGGTTTTC | TACGGGTTTTCGTGTT | | |
| | GTTTTTTCGTAGTTTTTAT | GGAAATTTTATCGTTA | | |
| | TTTAGTTTTTTGTTTTCGT | GTTTTATTGTTGTAAA | | |
| | ACGTTATGAAGTCGTCGTT | TAAAGTAATGATTTTT | | |
| | TTATCGTTGTTAGAATATT | TTAAAATATATATTTT | | |
| | ATTTTTGTGGAAAAAGGTA | TAATTAAAATATTTAT | | |
| | ATTCGGCGGTGATGGGCGG | TAAGATCGTATATACG | | |
| | GGTGTTTTTTAGTATCGGT | GGAGTTTTTGTATTTT | | |
| | TTTTTGGGTAATTTGTTGG | TTATATTGAGGTAGTT | | |
| | TTTTGGGGTTGTTGGCGCG | ATGGAGATTTTATATT | | |
| | TTCGGGGTTGGGGTGGTGT | G | | |
| | TCGCGGCGTTTATTGCGTT | | | |
| | CGTTGTTTTCGGTTTTTTA | | | |
| | TATGTTGGTGTGTGGTTTG | | | |
| | ACGGTTATCGATTTGTTGG | | | |
| | GTAAGTGTTTTTTAAGTTC | | | |
| | GGTGGTGTTGGTTGTTTAC | | | |
| | GTTTAGAATCGGAGTTTGC | | | |
| | GGGTGTTTGCGTTCGTATT | | | |
| | GGATAATTCGTTGTGTTAA | | | |
| | GTTTTCGTTTTTTTTATGT | | | |
| | TTTTTTTTGGGTTTTTTTC | | | |
| | GATATTGTAATTTTTGGTT | | | |
| | ATGGTATTGGAGTGTTGGT | | | |
| | ACOT11 | PCDHGA12 | CA3 | CHRNB1 |
| Probe | GAGTTTGGCTGGGGCTGGG | CGGAGATCCTGCTCGC | AGGCTGGCTGTCTGGC | CGCCCCCAGCGCCCCC |
| source | TGCCCAGTGGGCGGGCACA | CTTGCACGCGCCTGAA | TACGATCTCTGGACAC | AGCAGCATCAGCAGAG |
| sequence | GGCCCCTTGACG | GCACAAAGCAGATAGC | TTGTGCGAGTTTATTT | CCCCTGGGGTCATAGC |
| | | TA | CG | CT |

Bisulfite sequencing primers

| | | | | |
|---|---|---|---|---|
| Forward | TAGGAGTTTTGTATAGAAA | GATTGTGTAGTAATTG | TTATATGTTGTTTGTA | GAATAAGTGTAGTTTT |
| (5'-3') | GTTTT | GTTAGGATTT | AAGGGAGTT | GGTGTTTGGT |
| Reverse | ACCAACCCCCTTCCCTAA | ATTCTACCCTTAACCA | CTCCTTCCTCCATACA | ACTATCCTCCAACAAC |
| (5'-3') | | CTAAAATCAA | TTCTTA | AAATACACA |

MS-HRM primers

| | | | | |
|---|---|---|---|---|
| Forward | GTTTTGTATAGAAAGTTT | TGTTTATTAATCGGGG | TAAATAAACGAGTTTT | CGTTATTGTTTTTTCG |
| (5'-3') | TAGTGT | AGAGAAAA | TTTTAGTTTTTG | GGGTTTATATTTATT |
| Reverse | CCCAATAAACGAACACAA | CCGTTATTTCCACATA | CTAACTACGATCTCTA | CGAACTCCTCGTCACT |
| (5'-3') | ACC | CTCCCAA | AACACTTA | TCCCCTATACTA |

MS-HRM sequence

| | | | | |
|---|---|---|---|---|
| Untreated | GCCCTGCACAGAAAGCTC | GACTCTGAGCGCCGCT | CAAACAAACGAGTTCT | CGTCACTGCCCCTTCG |
| | CAGTGCCCGCCTAGCGGA | GTTCACCAATCGGGGA | TTCCAGCCTCTGTAAC | GGGCCTACACTTACCT |
| | GAGGAAGGACGAGGCTGC | GAGAAAAGCGGAGATC | CGGATCGCTAGAGCGA | GGGGCGAGCGGCGCCC |
| | CAGCTAGGCCACAGCCAC | CTGCTCGCCTTGCACG | AATAAACTCGCACAAG | CCAGCGCCCCCAGCAG |
| | GTCAAGGGGCCTGTGCCC | CGCCTGAAGCACAAAG | TGTCCAGAGATCGTAG | CATCAGCAGAGCCCCT |
| | GCCCACTGGG | CAGATAGCTAGGAATG | CCAG | GGGGTCATAGCCTGGC |
| | | AACCATCCCTGGGAGT | | GGCTCGCTCAGTGACT |
| | | ATGTGGAAACAACGG | | TCGCTCAGAGAGCCGC |
| | | | | TGGGACCGCCAGCACA |
| | | | | GGGGAAGTGACGAGGA |
| | | | | GCCG |
| Bisulfite | GTTTTGTATAGAAAGTTT | GATTTTAGCGTCGTTG | TAAATAAACGAGTTTT | CGTTATTGTTTTTTCG |
| converted | TAGTGTTCGTTTAGCGGA | TTTATTAATCGGGGAG | TTTTAGTTTTTGTAAT | GGGTTTATATTTATTT |
| | GAGGAAGGACGAGGTTGT | AGAAAAGCGGAGATTT | CGGATCGTTAGAGCGA | GGGGCGAGCGGCGTTT |
| | TAGTTAGGTTATAGTTAC | TGTTCGTTTTGTACGC | AATAAATTCGTATAAG | TTAGCGTTTTTAGTAG |
| | GTTAAGGGGTTTGTGTTC | GTTTGAAGTATAAAGT | TGTTTAGAGATCGTAG | TATTAGTAGAGTTTTT |
| | GTTTATTGGG | AGATAGTTAGGAATGA | TTAG | GGGGTTATAGTTTGGC |
| | | ATTATTTTTGGGAGTA | | GGTTCGTTTAGTGATT |
| | | TGTGGAAATAACGG | | TCGTTTAGAGAGTCGT |
| | | | | TGGGATCGTTAGTATA |
| | | | | GGGGAAGTGACGAGGA |
| | | | | GTTCG |

Sequenced sequence

| Untreated | TAGGAGCCCTGCACAGAA | GATTGTGCAGTAATTG | TTACATGTTGCCTGCA | GAATAAGTGCAGCCCT |
|---|---|---|---|---|
| | AGCTCCAGTGCCCGCCTA | GTTAGGACTCTGAGCG | AAGGGAGTCAAACTTA | GGTGCCTGGCCACGAC |
| | GCGGAGAGGAAGGACGAG | CCGCTGTTCACCAATC | GGGGGCAGGCAAACAA | CGCTGGCCCCGTCACT |
| | GCTGCCAGCTAGGCCACA | GGGGAGAGAAAAGCGG | ACGAGTTCTTTCCAGC | GCCCCTTCGGGGCCTA |
| | GCCACGTCAAGGGGCCTG | AGATCCTGCTCGCCTT | CTCTGTAACCGGATCG | CACTTACCTGGGGCGA |
| | TGCCCGCCCACTGGGCAC | GCACGCGCCTGAAGCA | CTAGAGCGAAATAAAC | GCGGCGCCCCCAGCGC |
| | CCAGCCCCAGCCAAACTC | CAAAGCAGATAGCTAG | TCGCACAAGTGTCCAG | CCCCAGCAGCATCAGC |
| | CAGGCACCCCCAGTCCCA | GAATGAACCATCCCTG | AGATCGTAGCCAGACA | AGAGCCCCTGGGGTCA |
| | GAGCTCATCATCCTGCCA | GGAGTATGTGGAAACA | GCCAGCCTGCGCTTGA | TAGCCTGGCGGCTCGC |
| | ACAGTGTCTCTTGGCTCT | ACGGAGGAGCTCTGAC | AGCAACTTTTAAGTGA | TCAGTGACTTCGCTCA |
| | GTGATCACTCCCAGGGAA | TTCCCAACTGTCCCAT | GGCTGCAAGAGCCGCC | GAGAGCCGCTGGGACC |
| | GGGGGCTGGT | TCTATGGGCGAAGGAA | GGGATGTAGATTTTAG | GCCAGCACAGGGGAAG |
| | | CTGCTCCTGACTTCAG | TTCGTGGCCAAGCACA | TGACGAGGAGCCCGGG |
| | | TGGTTAAGGGCAGAAT | ACTACGACACCCTGTC | AATGTGCACCTGTTGC |
| | | | CCTGCCCCCACCCCAT | TGGAGGACAGC |
| | | | CCCCAAGAATGCATGG | |
| | | | AGGAAGGAG | |
| Bisulfite | TAGGAGTTTTGTATAGAA | GATTGTGTAGTAATTG | TTATATGTTGTTTGTA | GAATAAGTGTAGTTTT |
| converted | AGTTTTAGTGTTCGTTTA | GTTAGGATTTTGAGCG | AAGGGAGTTAAATTTA | GGTGTTTGGTTACGAT |
| | GCGGAGAGGAAGGACGAG | TCGTTGTTTATTAATC | GGGGGTAGGTAAATAA | CGTTGGTTTCGTTATT |
| | GTTGTTAGTTAGGTTATA | GGGGAGAGAAAAGCGG | ACGAGTTTTTTTTAGT | GTTTTTTCGGGGTTTA |
| | GTTACGTTAAGGGGTTTG | AGATTTTGTTCGTTTT | TTTTGTAATCGGATCG | TATTTATTTGGGGCGA |
| | TGTTCGTTTATTGGGTAT | GTACGCGTTTGAAGTA | TTAGAGCGAAATAAAT | GCGGCGTTTTTAGCGT |
| | TTAGTTTTAGTTAAATTT | TAAAGTAGATAGTTAG | TCGTATAAGTGTTTAG | TTTTAGTAGTATTAGT |
| | TAGGTATTTTTAGTTTTA | GAATGAATTATTTTTG | AGATCGTAGTTAGATA | AGAGTTTTTGGGGTTA |
| | GAGTTTATTATTTTGTTA | GGAGTATGTGGAAATA | GTTAGTTTGCGTTTGA | TAGTTTGGCGGTTCGT |
| | ATAGTGTTTTTTGGTTTT | ACGGAGGAGTTTTTGA | AGTAATTTTTAAGTGA | TTAGTGATTTCGTTTA |
| | GTGATTATTTTTAGGGAA | TTTTTTAATTGTTTTA | GGTTGTAAGAGTCGTC | GAGAGTCGTTGGGATC |
| | GGGGGTTGGT | TTTTATGGGCGAAGGA | GGGATGTAGATTTTAG | GTTAGTATAGGGGAAG |
| | | ATTGTTTTTGATTTTA | TTCGTGGTTAAGTATA | TGACGAGGAGTTCGGG |
| | | GTGGTTAAGGGTAGAA | ATTACGATATTTTGTT | AATGTGTATTTGTTGT |
| | | T | TTTGTTTTTATTTTAT | TGGAGGATAGT |
| | | | TTTTAAGAATGTATGG | |
| | | | AGGAAGGAG | |
| | SOCS3 | SCARF2 | | |
| Probe | GGAAACTTGCTGTGGGTG | CGGTGGGGTGCTGTGG | | |
| source | ACCATGGCGCACGGAGCC | AGTTGGCTTTCGGCCT | | |
| sequence | AGCGTGGATCTGCG | CGACCTGGGCTGTCTG | | |
| | | CG | | |

Bisulfite sequencing primers

| | | |
|---|---|---|
| Forward | Not done | Not done |
| (5'-3') | | |
| Reverse | Not done | Not done |
| (5'-3') | | |

MS-HRM primers

| | | |
|---|---|---|
| Forward | GAGGTCGCGAAGTAGTTG | GGAATCGGTTAAGGGA |
| (5'-3') | TAGT | GTGG |
| Reverse | AAA ATC TTA AAA | CCGCACACCCCAACAC |
| (5'-3') | CGC AAA CTA ATA | TACAAA |
| | TCC AAA | |

MS-HRM sequence

| | | |
|---|---|---|
| Untreated | GAGGCCGCGAAGCAGCTG | GAACCGGCCAAGGGAG |
| | CAGCCGCCGCCGCGCAGA | TGGGGCCCGCAGACAG |
| | TCCACGCTGGCTCCGTGC | CCCAGGTCGAGGCCGA |
| | GCCATGGTCACCCACAGC | AAGCCAACTCCACAGC |
| | AAGTTTCCCGCCGCCGGG | ACCCCACCGCGAAGTC |
| | ATGAGCCGCCCCCTGGAC | CTTGTAGTGCTGGGGT |
| | ACCAGCCTGCGCCTCAAG | GTGCGG |
| | ACCTT | |
| Bisulfite | GAGGTCGCGAAGTAGTTG | GAATCGGTTAAGGGAG |
| converted | TAGTCGTCGTCGCGTAGA | TGGGGTTCGTAGATAG |
| | TTTACGTTGGTTTCGTGC | TTTAGGTCGAGGTCGA |
| | GTTATGGTTATTTATAGT | AAGTTAATTTTATAGT |
| | AAGTTTTTCGTCGTCGGG | ATTTTATCGCGAAGTT |
| | ATGAGTCGTTTTTTGGAT | TTTGTAGTGTTGGGGT |
| | ATTAGTTTGCGTTTTAAG | GTGCGG |
| | ATTTT | |

Sequenced sequence

| Untreated | | | | |
|---|---|---|---|---|
| Bisulfite | | | | |
| converted | | | | |
| | ZNF154 | HOXA9 | POU4F2 | EOMES |
| Probe | CGCCTTCGTGGCCCCAAC | CGGAAATTATGAAACT | AGCGGAGTCAGGCATC | GCGTCTGTAATTGCTT |
| source | TCGGCGCTCTGCTATCTC | GCAGATTTCATGTAAC | CGTTCAGACTGACAGC | ATTAACAGCGAATATT |
| sequence | TGATCCGGTGAACA | AACTTGGTGGCACCGG | AGAGGCGGCGAAGGAG | CAGGCTTCTCCTTATC |
| | | GG | CG | CG |

Bisulfite sequencing primers

| | | | | |
|---|---|---|---|---|
| Forward | TAAGAGGTTGGTGTAAAG | GGAGGTTGGTTTAGGG | GTTGGAGTTGGGAAGG | GTTGGAAAATTGGGTT |
| (5'-3') | GGTT | TTT | GT | GGAAAGT |
| Reverse | ATCCCTATCCCAAACCTA | CTTATTAAATAACTAT | ACATCCGTTCAAACTA | AAATTAAACTCCAACT |
| (5'-3') | AC | ACTTCCCC | ACAACAAAA | ACTTATTTCTTC |

MS-HRM primers

| | | | | |
|---|---|---|---|---|
| Forward | TGTGTTTATCGGATTAGA | GAGTTTACGTAGTAGT | GGGTTGTGCGAAGTTG | GGAAGTAGAGTTTCGA |
| (5'-3') | GATAGTAGAG | TGTTTAGGGTTT | AGTTTAT | TATTTTAGT |
| Reverse | CCAAACCTAACGTAAATC | CCCCCGATACCACCAA | ACATCCGTTCAAACTA | AATATTCGCTATTAAT |
| (5'-3') | CCCCAAAA | ATTATTACATA | ACAACAAAA | AAACAATTACAA |

MS-HRM sequence

| | | | | |
|---|---|---|---|---|
| Untreated | TGTGTTCACCGGATCAGA | GAGTCCACGTAGTAGT | GGGCTGTGCGAAGTTG | GGAAGCAGAGTCCCGA |
| | GATAGCAGAGCGCCGAGT | TGCCCAGGGCCCCAGT | AGCTCACCCGCCGCCG | CATCTCAGCCGGAAAA |
| | TGGGGCCACGAAGGCGTG | GGTGGCCATCACCGTG | CCTCCGGACTCTGTAC | TGCGCTCCCGGAGCGA |
| | AGGGGAGTCGTCGTCCCT | CCCAGCGCCTGGCCCG | GCCTGATCTCGGCTAC | TTACTGGCGGCGTCTG |
| | CCTGCACGAAAGCGTCTA | CCCGGCCCGACCCACG | GCGCTCCTTCGCCGCC | TAATTGCTTATTAACA |
| | AGCCTTGGCGACGCCGCC | GAAATTATGAAACTGC | TCTGCTGTCAGTCTGA | GCGAATATT |
| | CTGGGGGACCCACGTCAG | AGATTTCATGTAACAA | ACGGATGC | |
| | GCCTGG | CTTGGTGGCACCGGGG | | |
| | | G | | |
| Bisulfite | TGTGTTTATCGGATTAGA | GAGTTTACGTAGTAGT | GGGTTGTGCGAAGTTG | GGAAGTAGAGTTTCGA |
| converted | GATAGTAGAGCGTCGAGT | TGTTTAGGGTTTTAGT | AGTTTATTCGTCGTCG | TATTTTAGTCGGAAAA |
| | TGGGGTTACGAAGGCGTG | GGTGGTTATTATCGTG | TTTTCGGATTTTGTAC | TGCGTTTTCGGAGCGA |
| | AGGGGAGTCGTCGTTTTT | TTTAGCGTTTGGTTCG | GTTTGATTTCGGTTAC | TTATTGGCGGCGTTTG |
| | TTTGTACGAAAGCGTTTA | TTCGGTTCGATTTACG | GCGTTTTTTCGTCGTT | TAATTGTTTATTAATA |
| | AGTTTTGGCGACGTCGTT | GAAATTATGAAATTGT | TTTGTTGTTAGTTTGA | GCGAATATT |
| | TTGGGGGATTTACGTTAG | AGATTTTATGTAATAA | ACGGATGT | |
| | GTTTGG | TTTGGTGGTATCGGGG | | |
| | | G | | |

Sequenced sequence

| | | | | |
|---|---|---|---|---|
| Untreated | CAAGAGGTTGGTGCAAAG | GGAGGCTGGCCCAGGG | GCTGGAGCTGGGAAGG | GTTGGAAAACTGGGTT |
| | GGTCCCCGGCACCCACCT | TCCCCGGCGCATAGCG | GCTGTGCGAAGTTGAG | GGAAAGCTTCGCACTG |
| | CGGGATCTATGAAAACTA | GCCAACGCTCAGCTCA | CTCACCCGCCGCCGCC | TTCTACACTTGCGTGT |
| | CATTACCTAGAATGCTCT | TCCGCGGCGTCGGCGC | TCCGGACTCTGTACGC | GCGCACTCAGCAATCC |
| | GCGTTGAACGCCACGCTA | CCAGCAGGAACGAGTC | CTGATCTCGGCTACGC | TTTGGCCATCTCATCT |
| | CTAAGCCAGTAAGAGCTC | CACGTAGTAGTTGCCC | GCTCCTTCGCCGCCTC | GTTGTGGGCGAAGAGT |
| | AGAAAACCGACTTTCCTT | AGGGCCCCAGTGGTGG | TGCTGTCAGTCTGAAC | TTCCCGTGTGATCGCG |
| | GAGAGTCACAAAAAGAAA | CCATCACCGTGCCCAG | GGATGC | TTCGGTTGGGGAAGCA |
| | GGACGGGACTTTTGGGGG | CGCCTGGCCCGCCCGG | | GAGTCCCGACATCTCA |
| | GGCCTCTTCGTGGCGGCC | CCCGACCCACGGAAAT | | GCCGGAAAATGCGCTC |
| | ATTTTAGCTTCTCTGAGG | TATGAAACTGCAGATT | | CCGGAGCGATTACTGG |
| | TGTGTTCACCGGATCAGA | TCATGTAACAACTTGG | | CGGCGTCTGTAATTGC |
| | GATAGCAGAGCGCCGAGT | TGGCACCGGGGGGGAA | | TTATTAACAGCGAATA |
| | TGGGGCCACGAAGGCGTG | GTACAGTCACCTAATA | | TTCAGGCTTCTCCTTA |
| | AGGGGAGTCGTCGTCCCT | AG | | TCCGCAACGAAACGTG |
| | CCTGCACGAAAGCGTCTA | | | CCCCCCGCTTCCGTAA |
| | AGCCTTGGCGACGCCGCC | | | TAATGAAACGATAAAA |
| | CTGGGGGACCCACGTCAG | | | TATGACGGCCCCGCTC |
| | GCCTGGGATAGGGAC | | | TTGAATCTATCTGAGG |
| | | | | AAACGCAGCGAAGAAA |
| | | | | CAAGCAGCTGGAGTTT |
| | | | | AATTC |
| Bisulfite | TAAGAGGTTGGTGTAAAG | GGAGGTTGGTTTAGGG | GTTGGAGTTGGGAAGG | GTTGGAAAATTGGGTT |
| concerted | GGTTTTCGGTATTTATTT | TTTTCGGCGTATAGCG | GTTGTGCGAAGTTGAG | GGAAAGTTTCGTATTG |
| | CGGGATTTATGAAAATTA | GTTAACGTTTAGTTTA | TTTATTCGTCGTCGTT | TTTTATATTTGCGTGT |
| | TATTATTTAGAATGTTTT | TTCGCGGCGTCGGCGT | TTCGGATTTTGTACGT | GCGTATTTAGTAATTT |
| | GCGTTGAACGTTACGTTA | TTAGTAGGAACGAGTT | TTGATTTCGGTTACGC | TTTGGTTATTTTATTT |
| | TTAAGTTAGTAAGAGTTT | TACGTAGTAGTTGTTT | GTTTTTTCGTCGTTTT | GTTGTGGGCGAAGAGT |
| | AGAAAATCGATTTTTTTT | AGGGTTTTAGTGGTGG | TGTTGTTAGTTTGAAC | TTTTCGTGTGATCGCG |
| | GAGAGTTATAAAAAGAAA | TTATTATCGTGTTTAG | GGATGT | TTCGGTTGGGGAAGTA |
| | GGACGGGATTTTTGGGGG | CGTTTGGTTCGTTCGG | | GAGTTTCGATATTTTA |
| | GGTTTTTTCGTGGCGGTT | TTCGATTTACGGAAAT | | GTCGGAAAATGCGTTT |
| | ATTTTAGTTTTTTTGAGG | TATGAAATTGTAGATT | | TCGGAGCGATTATTGG |
| | TGTGTTTATCGGATTAGA | TTATGTAATAATTTGG | | CGGCGTTTGTAATTGT |
| | GATAGTAGAGCGTCGAGT | TGGTATCGGGGGGGAA | | TTATTAATAGCGAATA |
| | TGGGGTTACGAAGGCGTG | GTATAGTTATTTAATA | | TTTAGGTTTTTTTTTA |
| | AGGGGAGTCGTCGTTTTT | AG | | TTCGTAACGAAACGTG |
| | TTTGTACGAAAGCGTTTA | | | TTTTTCGTTTTCGTAA |
| | AGTTTTGGCGACGTCGTT | | | TAATGAAACGATAAAA |
| | TTGGGGGATTTACGTTAG | | | TATGACGGTTTCGTTT |
| | GTTTGGGATAGGGAT | | | TTGAATTTATTTGAGG |
| | | | | AAACGTAGCTTTGAGG |
| | | | | AAACGTAGCGAAGAAA |
| | | | | TAAGTAGTTGGAGTTT |
| | | | | AATTT |

What is claimed is:

1. A method for identifying in a subject or predicting a likelihood of a subject developing bladder cancer, comprising:
(a) collecting urine from a subject;
(b) assaying genomic material in the urine for one or more of the markers HOXA9, ZNF154, POU4F2, or EOMES being hypermethylated relative to the level of methylation in said markers in a control representative of a subject who is negative for bladder cancer; or relative to the level of methylation of the total genomic material in the assay; and
(c) wherein hypermethylation indicates bladder cancer in the subject.

2. The method of claim 1 wherein the determination is made by hybridizing the genomic material to an array of probes where the array is capable of determining the average percentage of methylation of the markers.

3. The method of claim 2 wherein bisulfite sequencing is also used in the determination of the average percentage of methylation of the markers.

4. The method of claim 3 wherein following bisulfite sequencing a high resolution melting analysis is performed.

5. The method of claim 1 further including determining whether any markers other than HOXA9, ZNF154, POU4F2, and EOMES are hypermethylated or hypomethylated in a tissue sample from the subject.

6. The method of claim 5 wherein the tissue sample is obtained by performing a cystoscopy on the patient.

7. The method of claim 5 wherein the markers are one or more of PTGDR; ZNF135; TBX4; ACOT11; PCDHGA12; CA3; CHRNB1; BRF1; SOCS3; PTGDR; or SCARF2.

8. The method of claim 7 wherein the one of more of the markers PTGDR; ZNF135; TBX4; ACOT11; PCDHGA12; or CA3 is hypermethylated relative to the level of methylation in said markers in the control; and/or one or more of the markers CHRNB1; BRF1; SOCS3; PTGDR; or SCARF2 is hypomethylated relative to the level of methylation in said markers in the control.

9. The method of claim 1 wherein hypermethylation of the markers is observed, and a monitoring program for the subject for bladder cancer development or progression is undertaken.

10. The method of claim 1 wherein hypermethylation of the markers is observed, and initiation of treatment, or a change in existing treatment regimens, is undertaken.

11. The method of claim 2 wherein the array is analyzed by establishing a threshold which reflects a significant level of methylation.

12. The method of claim 1 wherein the assaying includes amplification of portions of the markers HOXA9, ZNF154, POU4F2, or EOMES.

13. The method of claim 12 wherein the amplification step includes use of primers targeting the methylated or unmethylated portions of the markers.

14. A method for identifying bladder cancer in a subject comprising:
assaying genomic material in urine from the subject for one or more of the markers HOXA9, ZNF154, POU4F2, or EOMES being hypermethylated relative to the level of methylation in respective HOXA9, ZNF154, POU4F2, or EOMES non-bladder cancer control markers or relative to the level of methylation of total genomic material in the assay; and
wherein hypermethylation of one or more of the markers HOXA9, ZNF154, POU4F2, or EOMES indicates bladder cancer in the subject.

15. The method of claim 14 wherein the step of assaying includes hybridizing the genomic material to an array of probes where the array indicates the average percentage of methylation of the markers.

16. The method of claim 15 wherein the step of assaying includes bisulfite sequencing to determine the average percentage methylation of the markers.

17. The method of claim 16 wherein a high resolution melting analysis is performed following bisulfite sequencing.

18. The method of claim 14 further including determining whether markers other than HOXA9, ZNF154, POU4F2, and EOMES are hypermethylated or hypomethylated in a tissue sample from the subject.

19. The method of claim 18 wherein the tissue sample is obtained by performing a cystoscopy or transurethral resection of bladder tumor on the patient.

20. The method of claim 18 wherein the markers from the tissue sample are one or more of PTGDR; ZNF135; TBX4; ACOT11; PCDHGA12; CA3; CHRNB1; BRF1; SOCS3; PTGDR; or SCARF2.

21. The method of claim 20 wherein the one of more of the markers PTGDR; ZNF135; TBX4; ACOT11; PCDHGA12; or CA3 is hypermethylated relative to the level of methylation in respective control markers; or one or more of the markers CHRNB1; BRF1; SOCS3; PTGDR; or SCARF2 is hypomethylated relative to the level of methylation in respective control markers.

22. The method of claim 14 wherein hypermethylation of the markers is observed and the subject is monitored for bladder cancer development, recurrence or progression.

23. The method of claim 14 wherein hypermethylation of the markers is observed and the subject is treated for bladder cancer.

24. The method of claim 15 wherein the array is analyzed by establishing a threshold which reflects a significant level of methylation.

25. The method of claim 14 wherein the assaying includes amplification of portions of the markers HOXA9, ZNF154, POU4F2, or EOMES.

26. The method of claim 25 wherein the amplification step includes use of primers targeting the methylated or unmethylated portions of the markers.

27. The method of claim 14 wherein hypermethylation of two or more of the markers HOXA9, ZNF154, POU4F2, or EOMES indicates bladder cancer in the subject.

28. The method of claim 14 wherein hypermethylation of three or more of the markers HOXA9, ZNF154, POU4F2, or EOMES indicates bladder cancer in the subject.

29. The method of claim 14 wherein hypermethylation of the markers HOXA9, ZNF154, POU4F2, or EOMES indicates bladder cancer in the subject.

30. The method of claim 14 wherein the step of assaying further includes assaying for markers TWIST1 or VIM being hypermethylated relative to the level of methylation in respective TWIST1 or VIM non-bladder cancer control markers or relative to the level of methylation of total genomic material in the assay; and
wherein hypermethylation of one or more of the markers TWIST1 or VIM indicates bladder cancer in the subject.

31. A method for identifying bladder cancer in a subject comprising:
assaying genomic material in urine from the subject for marker HOXA9 being hypermethylated relative to the level of methylation of HOXA9 in a non-bladder cancer control sample or relative to the level of methylation of total genomic material in the assay; and
wherein hypermethylation of HOXA9 indicates bladder cancer in the subject.

32. A method for identifying bladder cancer in a subject comprising:
assaying genomic material in urine from the subject for marker ZNF154 being hypermethylated relative to the level of methylation of ZNF154 in a non-bladder cancer control sample or relative to the level of methylation of total genomic material in the assay; and
wherein hypermethylation of ZNF154 indicates bladder cancer in the subject.

33. A method for identifying bladder cancer in a subject comprising:
assaying genomic material in urine from the subject for marker POU4F2 being hypermethylated relative to the level of methylation of POU4F2 in a non-bladder cancer control sample or relative to the level of methylation of total genomic material in the assay; and
wherein hypermethylation of POU4F2 indicates bladder cancer in the subject.

34. A method for identifying bladder cancer in a subject comprising:
assaying genomic material in urine from the subject for marker EOMES being hypermethylated relative to the level of methylation of EOMES in a non-bladder cancer control sample or relative to the level of methylation of total genomic material in the assay; and
wherein hypermethylation of EOMES indicates bladder cancer in the subject.


SEQUENCE LISTING (186 sequences)

SEQ ID NO: 1 - DNA, 235 bp, Homo sapiens
gaataagtgcagccctggtgcctggccacgaccgctggccccgtcactgccccttcggggcctacacttacctggggcgagcggcgcccccagcgcccccagcagcatcagcagagcccctggggtcatagcctggcggctcgctcagtgacttcgctcagagagccgctgggaccgccagcacaggggaagtgacgaggagcccgggaatgtgcacctgttgctggaggacagc

SEQ ID NO: 2 - DNA, 36 bp, Artificial
  PCR primer
ggttaggtatagtggtttatatttgtaattttagta

SEQ ID NO: 3 - DNA, 27 bp, Artificial
  PCR primer
tttatcggattagagatagtagagcgt

SEQ ID NO: 4 - DNA, 23 bp, Artificial
  PCR primer
gttgtgcgaagttgagtttattc

SEQ ID NO: 5 - DNA, 23 bp, Artificial
  PCR primer
gtggttattatcgtgtttagcgt

SEQ ID NO: 6 - DNA, 23 bp, Artificial
  PCR primer
ggttggggaagtagagtttcgat

SEQ ID NO: 7 - DNA, 22 bp, Artificial
  PCR primer
gttagggttcgggggcgttgtt

SEQ ID NO: 8 - DNA, 20 bp, Artificial
  PCR primer
ttcgggagttagttcgcgtt

SEQ ID NO: 9 - DNA, 33 bp, Artificial
  PCR primer
attaactaaactaatcttaaactcctaacctca

SEQ ID NO: 10 - DNA, 24 bp, Artificial
  PCR primer
taacgtaaatcccccaaaacgacg

SEQ ID NO: 11 - DNA, 25 bp, Artificial
  PCR primer
ccgttcaaactaacaacaaaaacga

SEQ ID NO: 12 - DNA, 24 bp, Artificial
  PCR primer
ccgataccaccaaattattacata

SEQ ID NO: 13 - DNA, 23 bp, Artificial
  PCR primer
ataaacaattacaaacgccgcca

SEQ ID NO: 14 - DNA, 21 bp, Artificial
  PCR primer
ccgtcgccttcctccgacgaa

SEQ ID NO: 15 - DNA, 19 bp, Artificial
  PCR primer
accgccgaacatcctacga

SEQ ID NO: 16 - DNA, 16 bp, Artificial
  probe
cctaccttaacctccc

SEQ ID NO: 17 - DNA, 23 bp, Artificial
  probe
aacgacgactcccctcacgcctt

SEQ ID NO: 18 - DNA, 28 bp, Artificial
  probe
cggattttgtacgtttcatttcggttac

SEQ ID NO: 19 - DNA, 25 bp, Artificial
  probe
tggttcgttcggttcgatttacgga

SEQ ID NO: 20 - DNA, 26 bp, Artificial
  probe
cgctccgaaaacgcattttccgacta

SEQ ID NO: 21 - DNA, 22 bp, Artificial
  probe
cggcggggaaggaaatcgtttc

SEQ ID NO: 22 - DNA, 19 bp, Artificial
  probe
tcgtcgtttaggttatcgt

SEQ ID NO: 23 - DNA, 22 bp, Artificial
  PCR primer
taagaggttggtgtaaagggtt

SEQ ID NO: 24 - DNA, 20 bp, Artificial
  PCR primer
atccctatcccaaacctaac

SEQ ID NO: 25 - DNA, 19 bp, Artificial
  PCR primer
ggaggttggtttagggttt

SEQ ID NO: 26 - DNA, 24 bp, Artificial
  PCR primer
cttattaaataactatacttcccc

SEQ ID NO: 27 - DNA, 18 bp, Artificial
  PCR primer
gttggagttgggaagggt

SEQ ID NO: 28 - DNA, 25 bp, Artificial
  PCR primer
acatccgttcaaactaacaacaaaa

SEQ ID NO: 29 - DNA, 24 bp, Artificial
  PCR primer
taggagttttgtatagaaagtttt

SEQ ID NO: 30 - DNA, 18 bp, Artificial
  PCR primer
accaacccccttccctaa

SEQ ID NO: 31 - DNA, 23 bp, Artificial
  PCR primer
gttggaaaattgggttggaaagt

SEQ ID NO: 32 - DNA, 28 bp, Artificial
  PCR primer
aaattaaactccaactacttatttcttc

SEQ ID NO: 33 - DNA, 26 bp, Artificial
  PCR primer
gattgtgtagtaattggttaggattt

SEQ ID NO: 34 - DNA, 26 bp, Artificial
  PCR primer
attctacccttaaccactaaaatcaa

SEQ ID NO: 35 - DNA, 25 bp, Artificial
  PCR primer
ttatatgttgtttgtaaagggagtt

SEQ ID NO: 36 - DNA, 22 bp, Artificial
  PCR primer
ctccttcctccatacattctta

SEQ ID NO: 37 - DNA, 23 bp, Artificial
  PCR primer
gagtttagataggaggtttttgt

SEQ ID NO: 38 - DNA, 22 bp, Artificial
  PCR primer
accaacactccaataccataac

SEQ ID NO: 39 - DNA, 21 bp, Artificial
  PCR primer
gtgaggtttagttattaaagt

SEQ ID NO: 40 - DNA, 21 bp, Artificial
  PCR primer
taacaacatacaaaacatcac

SEQ ID NO: 41 - DNA, 19 bp, Artificial
  PCR primer
gaggtgggtatataggggt

SEQ ID NO: 42 - DNA, 27 bp, Artificial
  PCR primer
aatatcacttaaaataactccaactaa

SEQ ID NO: 43 - DNA, 23 bp, Artificial
  PCR primer
agatggggatattatattgggag

SEQ ID NO: 44 - DNA, 24 bp, Artificial
  PCR primer
ctcaaaaaaccaaccaaaacacta

SEQ ID NO: 45 - DNA, 26 bp, Artificial
  PCR primer
gaataagtgtagttttggtgtttggt

SEQ ID NO: 46 - DNA, 25 bp, Artificial
  PCR primer
actatcctccaacaacaaatacaca

SEQ ID NO: 47 - DNA, 28 bp, Artificial
  PCR primer
tgtgtttatcggattagagatagtagag

SEQ ID NO: 48 - DNA, 26 bp, Artificial
  PCR primer
ccaaacctaacgtaaatcccccaaaa

SEQ ID NO: 49 - DNA, 23 bp, Artificial
  PCR primer
gggttgtgcgaagttgagtttat

SEQ ID NO: 50 - DNA, 25 bp, Artificial
  PCR primer
acatccgttcaaactaacaacaaaa

SEQ ID NO: 51 - DNA, 28 bp, Artificial
  PCR primer
gagtttacgtagtagttgtttagggttt

SEQ ID NO: 52 - DNA, 27 bp, Artificial
  PCR primer
cccccgataccaccaaattattacata

SEQ ID NO: 53 - DNA, 24 bp, Artificial
  PCR primer
gttttgtatagaaagttttagtgt

SEQ ID NO: 54 - DNA, 21 bp, Artificial
  PCR primer
cccaataaacgaacacaaacc

SEQ ID NO: 55 - DNA, 25 bp, Artificial
  PCR primer
ggaagtagagtttcgatattttagt

SEQ ID NO: 56 - DNA, 28 bp, Artificial
  PCR primer
aatattcgctattaataaacaattacaa

SEQ ID NO: 57 - DNA, 24 bp, Artificial
  PCR primer
tgtttattaatcggggagagaaaa

SEQ ID NO: 58 - DNA, 23 bp, Artificial
  PCR primer
ccgttatttccacatactcccaa

SEQ ID NO: 59 - DNA, 28 bp, Artificial
  PCR primer
taaataaacgagttttttttagtttttg

SEQ ID NO: 60 - DNA, 24 bp, Artificial
  PCR primer
ctaactacgatctctaaacactta

SEQ ID NO: 61 - DNA, 24 bp, Artificial
  PCR primer
gagtgattcgttgtgtgttgtgtt

SEQ ID NO: 62 - DNA, 23 bp, Artificial
  PCR primer
acgttaactacgctaacctctcc

SEQ ID NO: 63 - DNA, 27 bp, Artificial
  PCR primer
agttattcgtggttatttgtggttatt

SEQ ID NO: 64 - DNA, 27 bp, Artificial
  PCR primer
cgcatttctttttaaactcattcctaa

SEQ ID NO: 65 - DNA, 31 bp, Artificial
  PCR primer
cgttattgttttttcggggtttatatttatt

SEQ ID NO: 66 - DNA, 28 bp, Artificial
  PCR primer
cgaactcctcgtcacttcccctatacta

SEQ ID NO: 67 - DNA, 30 bp, Artificial
  PCR primer
cgttttttcgtagtttttattttagttttt

SEQ ID NO: 68 - DNA, 23 bp, Artificial
  PCR primer
cgccgaattacctttttccacaa

SEQ ID NO: 69 - DNA, 20 bp, Artificial
  PCR primer
ggaatcggttaagggagtgg

SEQ ID NO: 70 - DNA, 22 bp, Artificial
  PCR primer
ccgcacaccccaacactacaaa

SEQ ID NO: 71 - DNA, 22 bp, Artificial
  PCR primer
gaggtcgcgaagtagttgtagt

SEQ ID NO: 72 - DNA, 30 bp, Artificial
  PCR primer
aaaatcttaaaacgcaaactaatatccaaa

SEQ ID NO: 73 - DNA, 22 bp, Artificial
  PCR primer
gcgttttaagcggtatttattt

SEQ ID NO: 74 - DNA, 27 bp, Artificial
  PCR primer
cgttcgtaaactctcaatactactaaa

SEQ ID NO: 75 - DNA, 27 bp, Artificial
  PCR primer
tttttggggtgcgtttttagtttaagg

SEQ ID NO: 76 - DNA, 27 bp, Artificial
  PCR primer
caaacttcgaaaaaacccctactccaa

SEQ ID NO: 77 - DNA, 50 bp, Artificial
  probe
cgccatgaagtcgccgttctaccgctgccagaacaccacctctgtggaaa

SEQ ID NO: 78 - DNA, 50 bp, Artificial
  probe
agtgactgggcctggtgaaacagggcgcgcggggtgcctgggcatcaacg

SEQ ID NO: 79 - DNA, 50 bp, Artificial
  probe
cgggcacagcacacaacgagtcactcgcccaggtccacactcgccagagg

SEQ ID NO: 80 - DNA, 50 bp, Artificial
  probe
cgccctcaaatatctgcaggtgctgttcacaatcgccatagggccggtga

SEQ ID NO: 81 - DNA, 23 bp, Artificial
  sequencing primer
gagtttagataggaggtttttgt

SEQ ID NO: 82 - DNA, 22 bp, Artificial
  Sequencing primer
gagtgattgggtttggtgaaat

SEQ ID NO: 83 - DNA, 22 bp, Artificial
  sequencing primer
accaacactccaataccataac

SEQ ID NO: 84 - DNA, 28 bp, Artificial
  sequencing primer
caatataaaatctccataactacctcaa

SEQ ID NO: 85 - DNA, 30 bp, Artificial; identical to SEQ ID NO: 67
  PCR primer

SEQ ID NO: 86 - DNA, 22 bp, Artificial
  PCR primer
gcgttttaagcggtatttattt

SEQ ID NO: 87 - DNA, 24 bp, Artificial
  PCR primer
gagtgattcgttgtgtgttgtgtt

SEQ ID NO: 88 - DNA, 27 bp, Artificial
  PCR primer
agttattcgtggttatttgtggttatt

SEQ ID NO: 89 - DNA, 23 bp, Artificial
  PCR primer
cgccgaattacctttttccacaa

SEQ ID NO: 90 - DNA, 27 bp, Artificial
  PCR primer
cgttcgtaaactctcaatactactaaa

SEQ ID NO: 91 - DNA, 23 bp, Artificial
  PCR primer
acgttaactacgctaacctctcc

SEQ ID NO: 92 - DNA, 27 bp, Artificial
  PCR primer
cgcatttctttttaaactcattcctaa

SEQ ID NO: 93 - DNA, 104 bp, Homo sapiens
cgcccttccgcagccttcactccagccctctgctcccgcacgccatgaagtcgccgttctaccgctgccagaacaccacctctgtggaaaaaggcaactcggcg

SEQ ID NO: 94 - DNA, 106 bp, Homo sapiens
gcgttccaagcggcacttatcccgcgttgatgcccaggcaccccgcgcgccctgtttcaccaggcccagtcactccagctccagcagcactgagagctcacgaacg

SEQ ID NO: 95 - DNA, 118 bp, Homo sapiens
gagtgactcgttgtgtgctgtgcccgcaggagatgctgcaggataagggcctgtccgagagcgaggaggccttccgggccccgggcccagcgctcggagaggccagcgcagccaacgc

SEQ ID NO: 96 - DNA, 161 bp, Homo sapiens
agtcattcgtggccacctgtggttacccgtgagtcacctcgctgtgccccctgcccagagcgggaaccctggctgcgcacgccctcaaatatctgcaggtgctgttcacaatcgccatagggccggtgacatacccaggaatgagcctaaaaagaaatgcg

SEQ ID NO: 97 - DNA, 104 bp, Homo sapiens
cgttttttcgtagtttttattttagttttttgttttcgtacgttatgaagtcgtcgttttatcgttgttagaatattatttttgtggaaaaaggtaattcggcg

SEQ ID NO: 98 - DNA, 106 bp, Homo sapiens
gcgttttaagcggtatttatttcgcgttgatgtttaggtatttcgcgcgttttgttttattaggtttagttattttagttttagtagtattgagagtttacgaacg

SEQ ID NO: 99 - DNA, 118 bp, Homo sapiens
gagtgattcgttgtgtgttgtgttcgtaggagatgttgtaggataagggtttgttcgagagcgaggaggtttttcgggtttcgggtttagcgttcggagaggttagcgtagttaacgt

SEQ ID NO: 100 - DNA, 161 bp, Homo sapiens
agttattcgtggttatttgtggttattcgtgagttatttcgttgtgttttttgtttagagcgggaattttggttgcgtacgtttttaaatatttgtaggtgttgtttataatcgttatagggtcggtgatatatttaggaatgagtttaaaaagaaatgcg

SEQ ID NO: 101 - DNA, 570 bp, Homo sapiens
gagtttagataggaggttcctgccgtggggaacaccccgccgccctcggagctttttctgtggcgcagcttctccgcccgagccgcgcgcggagctgccgggggctccttagcacccgggcgccggggccctcgcccttccgcagccttcactccagccctctgctcccgcacgccatgaagtcgccgttctaccgctgccagaacaccacctctgtggaaaaaggcaactcggcggtgatgggcggggtgctcttcagcaccggcctcctgggcaacctgctggccctggggctgctggcgcgctcggggctggggtggtgctcgcggcgtccactgcgcccgctgccctcggtcttctacatgctggtgtgtggcctgacggtcaccgacttgctgggcaagtgcctcctaagcccggtggtgctggctgcctacgctcagaaccggagtctgcgggtgcttgcgcccgcattggacaactcgttgtgccaagccttcgccttcttcatgtccttctttgggctctcctcgacactgcaactcctggccatggcactggagtgctggc

SEQ ID NO: 102 - DNA, 257 bp, Homo sapiens
gagtgactgggcctggtgaaacagggcgcgcggggtgcctgggcatcaacgcgggataagtgccgcttggaacgccgtgagctcccggcgcgaccacacgggctttcgtgttggaaactctaccgtcagttttactgctgcaaacaaagcaatgattttttcaaaacacatactttcaaccaaaacattcaccaagatcgcacacacgggagtctttgcattttccacactgaggcagccatggagacttcacactg

SEQ ID NO: 103 - DNA, 570 bp, Homo sapiens
gagtttagataggaggtttttgtcgtggggaatatttcgtcgttttcggagttttttttgtggcgtagttttttcgttcgagtcgcgcgcggagttgtcgggggttttttagtattcgggcgtcggggttttcgttttttcgtagtttttattttagttttttgttttcgtacgttatgaagtcgtcgttttatcgttgttagaatattatttttgtggaaaaaggtaattcggcggtgatgggcggggtgttttttagtatcggttttttgggtaatttgttggttttggggttgttggcgcgttcggggttggggtggtgttcgcggcgtttattgcgttcgttgttttcggttttttatatgttggtgtgtggtttgacggttatcgatttgttgggtaagtgttttttaagttcggtggtgttggttgtttacgtttagaatcggagtttgcgggtgtttgcgttcgtattggataattcgttgtgttaagttttcgttttttttatgtttttttttgggtttttttcgatattgtaatttttggttatggtattggagtgttggt

SEQ ID NO: 104 - DNA, 257 bp, Homo sapiens
gagtgattgggtttggtgaaatagggcgcgcggggtgtttgggtattaacgcgggataagtgtcgtttggaacgtcgtgagttttcggcgcgattatacgggttttcgtgttggaaattttatcgttagttttattgttgtaaataaagtaatgatttttttaaaatatatatttttaattaaaatatttattaagatcgtatatacgggagtttttgtattttttatattgaggtagttatggagattttatattg

SEQ ID NO: 105 - DNA, 50 bp, Artificial
  probe
gagtttggctggggctgggtgcccagtgggcgggcacaggccccttgacg

SEQ ID NO: 106 - DNA, 50 bp, Artificial
  probe
cggagatcctgctcgccttgcacgcgcctgaagcacaaagcagatagcta

SEQ ID NO: 107 - DNA, 50 bp, Artificial
  probe
aggctggctgtctggctacgatctctggacacttgtgcgagtttatttcg

SEQ ID NO: 108 - DNA, 50 bp, Artificial
  probe
cgcccccagcgcccccagcagcatcagcagagcccctggggtcatagcct

SEQ ID NO: 109 - DNA, 24 bp, Artificial
  sequencing primer
taggagttttgtatagaaagtttt

SEQ ID NO: 110 - DNA, 26 bp, Artificial
  sequencing primer
gattgtgtagtaattggttaggattt

SEQ ID NO: 111 - DNA, 25 bp, Artificial
  Sequencing primer
ttatatgttgtttgtaaagggagtt

SEQ ID NO: 112 - DNA, 26 bp, Artificial
  sequencing primer
gaataagtgtagttttggtgtttggt

SEQ ID NO: 113 - DNA, 18 bp, Artificial
  sequencing primer
accaacccccttccctaa

SEQ ID NO: 114 - DNA, 26 bp, Artificial
  sequencing primer
attctacccttaaccactaaaatcaa

SEQ ID NO: 115 - DNA, 22 bp, Artificial
  Sequencing primer
ctccttcctccatacattctta

SEQ ID NO: 116 - DNA, 25 bp, Artificial
  sequencing primer
actatcctccaacaacaaatacaca

SEQ ID NO: 117 - DNA, 24 bp, Artificial
  PCR primer
gttttgtatagaaagttttagtgt

SEQ ID NO: 118 - DNA, 24 bp, Artificial
  PCR primer
tgtttattaatcggggagagaaaa

SEQ ID NO: 119 - DNA, 28 bp, Artificial
  PCR primer
taaataaacgagttttttttagtttttg

SEQ ID NO: 120 - DNA, 31 bp, Artificial; identical to SEQ ID NO: 65
  PCR primer

SEQ ID NO: 121 - DNA, 21 bp, Artificial
  PCR primer
cccaataaacgaacacaaacc

SEQ ID NO: 122 - DNA, 23 bp, Artificial
  PCR primer
ccgttatttccacatactcccaa

SEQ ID NO: 123 - DNA, 24 bp, Artificial
  PCR primer
ctaactacgatctctaaacactta

SEQ ID NO: 124 - DNA, 28 bp, Artificial
  PCR primer
cgaactcctcgtcacttcccctatacta

SEQ ID NO: 125 - DNA, 100 bp, Homo sapiens
gccctgcacagaaagctccagtgcccgcctagcggagaggaaggacgaggctgccagctaggccacagccacgtcaaggggcctgtgcccgcccactggg

SEQ ID NO: 126 - DNA, 127 bp, Homo sapiens
gactctgagcgccgctgttcaccaatcggggagagaaaagcggagatcctgctcgccttgcacgcgcctgaagcacaaagcagatagctaggaatgaaccatccctgggagtatgtggaaacaacgg

SEQ ID NO: 127 - DNA, 84 bp, Homo sapiens
caaacaaacgagttctttccagcctctgtaaccggatcgctagagcgaaataaactcgcacaagtgtccagagatcgtagccag

SEQ ID NO: 128 - DNA, 165 bp, Homo sapiens
cgtcactgccccttcggggcctacacttacctggggcgagcggcgcccccagcgcccccagcagcatcagcagagcccctggggtcatagcctggcggctcgctcagtgacttcgctcagagagccgctgggaccgccagcacaggggaagtgacgaggagcccg

SEQ ID NO: 129 - DNA, 100 bp, Homo sapiens
gttttgtatagaaagttttagtgttcgtttagcggagaggaaggacgaggttgttagttaggttatagttacgttaaggggtttgtgttcgtttattggg

SEQ ID NO: 130 - DNA, 127 bp, Homo sapiens
gattttgagcgtcgttgtttattaatcggggagagaaaagcggagattttgttcgttttgtacgcgtttgaagtataaagtagatagttaggaatgaattatttttgggagtatgtggaaataacgg

SEQ ID NO: 131 - DNA, 84 bp, Homo sapiens
taaataaacgagttttttttagtttttgtaatcggatcgttagagcgaaataaattcgtataagtgtttagagatcgtagttag

SEQ ID NO: 132 - DNA, 165 bp, Homo sapiens
cgttattgttttttcggggtttatatttatttggggcgagcggcgtttttagcgtttttagtagtattagtagagtttttggggttatagtttggcggttcgtttagtgatttcgtttagagagtcgttgggatcgttagtataggggaagtgacgaggagttcg

SEQ ID NO: 133 - DNA, 208 bp, Homo sapiens
taggagccctgcacagaaagctccagtgcccgcctagcggagaggaaggacgaggctgccagctaggccacagccacgtcaaggggcctgtgcccgcccactgggcacccagccccagccaaactccaggcacccccagtcccagagctcatcatcctgccaacagtgtctcttggctctgtgatcactcccagggaagggggctggt

SEQ ID NO: 134 - DNA, 224 bp, Homo sapiens
gattgtgcagtaattggttaggactctgagcgccgctgttcaccaatcggggagagaaaagcggagatcctgctcgccttgcacgcgcctgaagcacaaagcagatagctaggaatgaaccatccctgggagtatgtggaaacaacggaggagctctgacttcccaactgtcccattctatgggcgaaggaactgctcctgacttcagtggttaagggcagaat

SEQ ID NO: 135 - DNA, 265 bp, Homo sapiens
ttacatgttgcctgcaaagggagtcaaacttagggggcaggcaaacaaacgagttctttccagcctctgtaaccggatcgctagagcgaaataaactcgcacaagtgtccagagatcgtagccagacagccagcctgcgcttgaagcaacttttaagtgaggctgcaagagccgccgggatgtagattttagttcgtggccaagcacaactacgacaccctgtccctgcccccaccccatccccaagaatgcatggaggaaggag

SEQ ID NO: 136 - DNA, 235 bp, Homo sapiens; identical to SEQ ID NO: 1

SEQ ID NO: 137 - DNA, 208 bp, Homo sapiens
taggagttttgtatagaaagttttagtgttcgtttagcggagaggaaggacgaggttgttagttaggttatagttacgttaaggggtttgtgttcgtttattgggtatttagttttagttaaattttaggtatttttagttttagagtttattattttgttaatagtgttttttggttttgtgattatttttagggaagggggttggt

SEQ ID NO: 138 - DNA, 224 bp, Homo sapiens
gattgtgtagtaattggttaggattttgagcgtcgttgtttattaatcggggagagaaaagcggagattttgttcgttttgtacgcgtttgaagtataaagtagatagttaggaatgaattatttttgggagtatgtggaaataacggaggagttttgattttttaattgttttattttatgggcgaaggaattgtttttgattttagtggttaagggtagaat

SEQ ID NO: 139 - DNA, 265 bp, Homo sapiens
ttatatgttgtttgtaaagggagttaaatttagggggtaggtaaataaacgagttttttttagtttttgtaatcggatcgttagagcgaaataaattcgtataagtgtttagagatcgtagttagatagttagtttgcgtttgaagtaatttttaagtgaggttgtaagagtcgtcgggatgtagattttagttcgtggttaagtataattacgatattttgtttttgtttttattttatttttaagaatgtatggaggaaggag

SEQ ID NO: 140 - DNA, 235 bp, Homo sapiens
gaataagtgtagttttggtgtttggttacgatcgttggtttcgttattgttttttcggggtttatatttatttggggcgagcggcgtttttagcgtttttagtagtattagtagagtttttggggttatagtttggcggttcgtttagtgatttcgtttagagagtcgttgggatcgttagtataggggaagtgacgaggagttcgggaatgtgtatttgttgttggaggatagt

SEQ ID NO: 141 - DNA, 22 bp, Artificial
  PCR primer
gaggtcgcgaagtagttgtagt

SEQ ID NO: 142 - DNA, 20 bp, Artificial
  PCR primer
ggaatcggttaagggagtgg

SEQ ID NO: 143 - DNA, 30 bp, Artificial; identical to SEQ ID NO: 72
  PCR primer

SEQ ID NO: 144 - DNA, 22 bp, Artificial
  PCR primer
ccgcacaccccaacactacaaa

SEQ ID NO: 145 - DNA, 131 bp, Homo sapiens
gaggccgcgaagcagctgcagccgccgccgcgcagatccacgctggctccgtgcgccatggtcacccacagcaagtttcccgccgccgggatgagccgccccctggacaccagcctgcgcctcaagacctt

SEQ ID NO: 146 - DNA, 102 bp, Homo sapiens
gaaccggccaagggagtggggcccgcagacagcccaggtcgaggccgaaagccaactccacagcaccccaccgcgaagtccttgtagtgctggggtgtgcgg

SEQ ID NO: 147 - DNA, 131 bp, Homo sapiens
gaggtcgcgaagtagttgtagtcgtcgtcgcgtagatttacgttggtttcgtgcgttatggttatttatagtaagtttttcgtcgtcgggatgagtcgttttttggatattagtttgcgttttaagatttt

SEQ ID NO: 148 - DNA, 102 bp, Homo sapiens
gaatcggttaagggagtggggttcgtagatagtttaggtcgaggtcgaaagttaattttatagtattttatcgcgaagtttttgtagtgttggggtgtgcgg

SEQ ID NO: 149 - DNA, 50 bp, Artificial
  probe
cgccttcgtggccccaactcggcgctctgctatctctgatccggtgaaca

SEQ ID NO: 150 - DNA, 50 bp, Artificial
  probe
cggaaattatgaaactgcagatttcatgtaacaacttggtggcaccgggg

SEQ ID NO: 151 - DNA, 50 bp, Artificial
  probe
agcggagtcaggcatccgttcagactgacagcagaggcggcgaaggagcg

SEQ ID NO: 152 - DNA, 50 bp, Artificial
  probe
gcgtctgtaattgcttattaacagcgaatattcaggcttctccttatccg

SEQ ID NO: 153 - DNA, 22 bp, Artificial
  sequencing primer
taagaggttggtgtaaagggtt

SEQ ID NO: 154 - DNA, 19 bp, Artificial
  sequencing primer
ggaggttggtttagggttt

SEQ ID NO: 155 - DNA, 18 bp, Artificial
  sequencing primer
gttggagttgggaagggt

SEQ ID NO: 156 - DNA, 23 bp, Artificial
  sequencing primer
gttggaaaattgggttggaaagt

SEQ ID NO: 157 - DNA, 20 bp, Artificial
  sequencing primer
atccctatcccaaacctaac

SEQ ID NO: 158 - DNA, 24 bp, Artificial
  sequencing primer
cttattaaataactatacttcccc

SEQ ID NO: 159 - DNA, 25 bp, Artificial
  sequencing primer
acatccgttcaaactaacaacaaaa

SEQ ID NO: 160 - DNA, 28 bp, Artificial
  sequencing primer
aaattaaactccaactacttatttcttc

SEQ ID NO: 161 - DNA, 28 bp, Artificial
  PCR primer
tgtgtttatcggattagagatagtagag

SEQ ID NO: 162 - DNA, 28 bp, Artificial
  PCR primer
gagtttacgtagtagttgtttagggttt

SEQ ID NO: 163 - DNA, 23 bp, Artificial
  PCR primer
gggttgtgcgaagttgagtttat

SEQ ID NO: 164 - DNA, 25 bp, Artificial
  PCR primer
ggaagtagagtttcgatattttagt

SEQ ID NO: 165 - DNA, 26 bp, Artificial
  PCR primer
ccaaacctaacgtaaatcccccaaaa

SEQ ID NO: 166 - DNA, 27 bp, Artificial
  PCR primer
cccccgataccaccaaattattacata

SEQ ID NO: 167 - DNA, 25 bp, Artificial
  PCR primer
acatccgttcaaactaacaacaaaa

SEQ ID NO: 168 - DNA, 28 bp, Artificial
  PCR primer
aatattcgctattaataaacaattacaa

SEQ ID NO: 169 - DNA, 132 bp, Homo sapiens
tgtgttcaccggatcagagatagcagagcgccgagttggggccacgaaggcgtgaggggagtcgtcgtccctcctgcacgaaagcgtctaagccttggcgacgccgccctgggggacccacgtcaggcctgg

SEQ ID NO: 170 - DNA, 129 bp, Homo sapiens
gagtccacgtagtagttgcccagggccccagtggtggccatcaccgtgcccagcgcctggcccgcccggcccgacccacggaaattatgaaactgcagatttcatgtaacaacttggtggcaccggggg

SEQ ID NO: 171 - DNA, 104 bp, Homo sapiens
gggctgtgcgaagttgagctcacccgccgccgcctccggactctgtacgcctgatctcggctacgcgctccttcgccgcctctgctgtcagtctgaacggatgc

SEQ ID NO: 172 - DNA, 89 bp, Homo sapiens
ggaagcagagtcccgacatctcagccggaaaatgcgctcccggagcgattactggcggcgtctgtaattgcttattaacagcgaatatt

SEQ ID NO: 173 - DNA, 132 bp, Homo sapiens
tgtgtttatcggattagagatagtagagcgtcgagttggggttacgaaggcgtgaggggagtcgtcgttttttttgtacgaaagcgtttaagttttggcgacgtcgttttgggggatttacgttaggtttgg

SEQ ID NO: 174 - DNA, 129 bp, Homo sapiens
gagtttacgtagtagttgtttagggttttagtggtggttattatcgtgtttagcgtttggttcgttcggttcgatttacggaaattatgaaattgtagattttatgtaataatttggtggtatcggggg

SEQ ID NO: 175 - DNA, 104 bp, Homo sapiens
gggttgtgcgaagttgagtttattcgtcgtcgttttcggattttgtacgtttgatttcggttacgcgttttttcgtcgtttttgttgttagtttgaacggatgt

SEQ ID NO: 176 - DNA, 89 bp, Homo sapiens
ggaagtagagtttcgatattttagtcggaaaatgcgttttcggagcgattattggcggcgtttgtaattgtttattaatagcgaatatt

SEQ ID NO: 177 - DNA, 339 bp, Homo sapiens
caagaggttggtgcaaagggtccccggcacccacctcgggatctatgaaaactacattacctagaatgctctgcgttgaacgccacgctactaagccagtaagagctcagaaaaccgactttccttgagagtcacaaaaagaaaggacgggacttttgggggggcctcttcgtggcggccattttagcttctctgaggtgtgttcaccggatcagagatagcagagcgccgagttggggccacgaaggcgtgaggggagtcgtcgtccctcctgcacgaaagcgtctaagccttggcgacgccgccctgggggacccacgtcaggcctgggatagggac

SEQ ID NO: 178 - DNA, 226 bp, Homo sapiens
ggaggctggcccagggtccccggcgcatagcggccaacgctcagctcatccgcggcgtcggcgcccagcaggaacgagtccacgtagtagttgcccagggccccagtggtggccatcaccgtgcccagcgcctggcccgcccggcccgacccacggaaattatgaaactgcagatttcatgtaacaacttggtggcaccgggggggaagtacagtcacctaataag

SEQ ID NO: 179 - DNA, 118 bp, Homo sapiens
gctggagctgggaagggctgtgcgaagttgagctcacccgccgccgcctccggactctgtacgcctgatctcggctacgcgctccttcgccgcctctgctgtcagtctgaacggatgc

SEQ ID NO: 180 - DNA, 341 bp, Homo sapiens
gttggaaaactgggttggaaagcttcgcactgttctacacttgcgtgtgcgcactcagcaatcctttggccatctcatctgttgtgggcgaagagtttcccgtgtgatcgcgttcggttggggaagcagagtcccgacatctcagccggaaaatgcgctcccggagcgattactggcggcgtctgtaattgcttattaacagcgaatattcaggcttctccttatccgcaacgaaacgtgccccccgcttccgtaataatgaaacgataaaatatgacggccccgctcttgaatctatctgaggaaacgcagcgaagaaacaagcagctggagtttaattc

SEQ ID NO: 181 - DNA, 339 bp, Homo sapiens
taagaggttggtgtaaagggttttcggtatttatttcgggatttatgaaaattatattatttagaatgttttgcgttgaacgttacgttattaagttagtaagagtttagaaaatcgattttttttgagagttataaaaagaaaggacgggatttttgggggggttttttcgtggcggttattttagtttttttgaggtgtgtttatcggattagagatagtagagcgtcgagttggggttacgaaggcgtgaggggagtcgtcgttttttttgtacgaaagcgtttaagttttggcgacgtcgttttgggggatttacgttaggtttgggatagggat

SEQ ID NO: 182 - DNA, 226 bp, Homo sapiens
ggaggttggtttagggttttcggcgtatagcggttaacgtttagtttattcgcggcgtcggcgtttagtaggaacgagtttacgtagtagttgtttagggttttagtggtggttattatcgtgtttagcgtttggttcgttcggttcgatttacggaaattatgaaattgtagattttatgtaataatttggtggtatcgggggggaagtatagttatttaataag

SEQ ID NO: 183 - DNA, 118 bp, Homo sapiens
gttggagttgggaagggttgtgcgaagttgagtttattcgtcgtcgttttcggattttgtacgtttgatttcggttacgcgttttttcgtcgtttttgttgttagtttgaacggatgt

SEQ ID NO: 184 - DNA, 341 bp, Homo sapiens
gttggaaaattgggttggaaagtttcgtattgttttatatttgcgtgtgcgtatttagtaattttttggttattttatttgttgtgggcgaagagtttttcgtgtgatcgcgttcggttggggaagtagagtttcgatattttagtcggaaaatgcgttttcggagcgattattggcggcgtttgtaattgtttattaatagcgaatatttaggtttttttttattcgtaacgaaacgtgtttttcgttttcgtaataatgaaacgataaaatatgacggtttcgtttttgaatttatttgaggaaacgtagcgaagaaataagtagttggagtttaattt

SEQ ID NO: 185 - DNA, 50 bp, Artificial
  probe
ggaaacttgctgtgggtgaccatggcgcacggagccagcgtggatctgcg

SEQ ID NO: 186 - DNA, 50 bp, Artificial
  probe
cggtggggtgctgtggagttggctttcggcctcgacctgggctgtctgcg